SEQUENCE LISTING (46 sequences)

SEQ ID NO: 1 - protein, 1273 aa, Severe acute respiratory syndrome coronavirus 2
  SITE (1)..(1273): SARS-Cov-2 spike (S) protein (including leader sequence) with S1 and S2 subunits; Genbank ACCESSION   QHD43416
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1198 aa, Severe acute respiratory syndrome coronavirus 2
  SITE (1)..(1198): SARS-Cov-2 spike protein without leader sequence, that includes the S1 subunit and the S2 subunit up to amino acid 1213 (amino acids 16-1213 of SEQ ID NO1)
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 3 - protein, 685 aa, Severe acute respiratory syndrome coronavirus 2
  SITE (1)..(685): SARS-Cov-2 spike S1 subunit (including leader sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 4 - protein, 670 aa, Severe acute respiratory syndrome coronavirus 2
  SITE (1)..(670): SARS-Cov-2 spike S1 subunit (no leader sequence), Amino acids 16-685 of SEQ ID NO1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 5 - protein, 219 aa, Severe acute respiratory syndrome coronavirus 2
  SITE (1)..(219): SARS-Cov-2 spike protein RBD, amino acids 319- 537 of SEQ ID NO1
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 6 - protein, 117 aa, Artificial Sequence
  Synthetic mAb S1D2   VH
EVQLVESGGGLIQPGGSLRLSCAASGFTVSSNYMSWVRQAPGKGLEWVSIIYPGGSTNYADSVKGRFTISRDNSRNTLYLQMNSLRAEDTAVYYCARELGYYGMDVWGQGTTVTVSS

SEQ ID NO: 7 - protein, 107 aa, Artificial Sequence
  Synthetic mAb S1D2   VL
DIQMTQSPSSVSASVGDRVTITCRASQGISTWLVWYQQKPGKAPNLLIYGASSLQSGVPSRFSGSGSGTDFTLTISSLQPEDFATYYCQQANSFPYTFGQGTKLEIK

SEQ ID NO: 8 - protein, 5 aa, Artificial Sequence
  Synthetic mA S1D2 Heavy chain CDR1
SNYMS

SEQ ID NO: 9 - protein, 16 aa, Artificial Sequence
  Synthetic mA S1D2 Heavy chain CDR2
IIYPGGSTNYADSVKG

SEQ ID NO: 10 - protein, 9 aa, Artificial Sequence
  Synthetic mA S1D2 Heavy chain CDR3
ELGYYGMDV

SEQ ID NO: 11 - protein, 11 aa, Artificial Sequence
  Synthetic mA S1D2 Light chain CDR1
RASQGISTWLV

SEQ ID NO: 12 - protein, 7 aa, Artificial Sequence
  Synthetic mA S1D2 Light chain CDR2
GASSLQS

SEQ ID NO: 13 - protein, 9 aa, Artificial Sequence
  Synthetic mA S1D2 Light chain CDR3
QQANSFPYT

SEQ ID NO: 14 - protein, 330 aa, Homo sapiens
  SITE (1)..(330): Human Fc region with LALA mutations (L234A, L235A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Homo sapiens
  SITE (1)..(330): Human Fc region with triple mutation YTE M252Y, S254T, T256E
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, Homo sapiens
  SITE (1)..(330): Human Fc region with LALA and YTE mutations (L234A, L235A) (M252Y, S254T, T256E)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 19 aa, Mus musculus
  SITE (1)..(19): mouse Ig gamma leader peptide sequence
MEWSWVFLFFLSVTTGVHS

SEQ ID NO: 18 - protein, 9 aa, Artificial Sequence
  Synthetic lower hinge/CH2 sequence
PAPELLGGP

SEQ ID NO: 19 - protein, 12 aa, Artificial Sequence
  Synthetic Fc region sequence (CH2)
SVFLFPPKPKDT

SEQ ID NO: 20 - protein, 23 aa, Artificial Sequence
  Synthetic hinge region
EPKSCDKTHTCPPCPAPELLGGP

SEQ ID NO: 21 - protein, 15 aa, Artificial Sequence
  Synthetic linker
GGGGSGGGGSGGGGS

SEQ ID NO: 22 - protein, 805 aa, Homo sapiens [sequence omitted]

SEQ ID NO: 23 - protein, 709 aa, Homo sapiens
  SITE (1)..(709): ACE2 receptor polypeptide soluble ectodomain, no signal peptide (amino acids 18-740)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLG

SEQ ID NO: 24 - protein, 598 aa, Homo sapiens
  SITE (1)..(598): ACE2 receptor polypeptide soluble ectodomain, no signal peptide (amino acids 18-615 )
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 25 - protein, 231 aa, Homo sapiens
  SITE (1)..(231): human IgG1 Fc region (amino acids 100-330 of Genbank P01857) (Fc tag)
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 508 bp, Cytomegalovirus
  misc_feature (1)..(508): CMV enhancer plus promoter
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 27 - protein, 676 aa, Severe acute respiratory syndrome coronavirus 2
  SITE (1)..(676): SARS-Cov-2 spike S1 subunit (no leader sequence), polyhistidine tag
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARHHHHHH

SEQ ID NO: 28 - protein, 117 aa, Artificial Sequence
  Synthetic S1D7270 antibody, Heavy Chain Variable region
EVQLVESGGGLIQPGGSLRLSCAASGFTVSSNYMSWVRQAPGKGLEWVSIIYPGGSTEYADSVKGRFTISRDNSRNTLYLQMNSLRAEDTAVYYCARELGYYGMDVWGQGTTVTVSS

SEQ ID NO: 29 - protein, 107 aa, Artificial Sequence
  Synthetic S1D7270 antibody, Light Chain Variable region
DIQMTQSPSSVSASVGDRVTITCRASQGISTWLVWYQQKPGKAPNLLIYGASSLQSGVPSRFSGSGSGTDFTLTISSLQPEDFATYYCQQANAYPYTFGQGTKLEIK

SEQ ID NO: 30 - protein, 5 aa, Artificial Sequence
  Synthetic S1D7270 antibody, Heavy Chain CDR1
SNYMS

SEQ ID NO: 31 - protein, 16 aa, Artificial Sequence
  Synthetic S1D7270 antibody, Heavy Chain CDR2
IIYPGGSTEYADSVKG

SEQ ID NO: 32 - protein, 9 aa, Artificial Sequence
  Synthetic S1D7270 antibody, Heavy Chain CDR3
ELGYYGMDV

SEQ ID NO: 33 - protein, 11 aa, Artificial Sequence
  Synthetic S1D7270 antibody, Light Chain CDR1
RASQGISTWLV

SEQ ID NO: 34 - protein, 7 aa, Artificial Sequence
  Synthetic S1D7270 antibody, Light Chain CDR2
GASSLQS

SEQ ID NO: 35 - protein, 9 aa, Artificial Sequence
  Synthetic S1D7270 antibody, Light Chain CDR3
QQANAYPYT

SEQ ID NO: 36 - DNA, 6089 bp, Artificial Sequence
  Synthetic Plasmid pNP-Muscle-S1D7270/HCLC, Expression vector includes genes for S1D7270 heavy and light chains, desmin promoters
ctagatccgctctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacaggggtaccgtcgacgccgccaccatgagtgtacctacccaggttttgggactgctgttgctctggctcacagacgcaaggtgcgacatccagatgacacagtcaccctctagtgtgtcagccagtgtgggtgacagagtcaccatcacctgccgcgcatctcagggaattagtacttggctggtctggtatcaacagaagcctggaaaagcccctaacctccttatttatggagcctctagcctccagagtggcgtaccttcacggttttccggttccggcagtggcacagacttcaccctgactatctctagcctgcagcccgaagatttcgctacctattattgccaacaggcaaacgcctacccctataccttcggccaaggcacaaaactggagattaagcgtacagtggccgcacctagcgtatttattttccccccatccgatgaacagctcaaatcagggaccgctagcgtggtgtgcttgctgaataatttctaccctagggaggctaaggtccagtggaaagtcgacaacgcactccagagcggtaatagccaggaaagtgtgaccgagcaggactctaaagattccacatactccctctcaagcaccctcaccttgtctaaggccgactatgagaagcacaaagtgtacgcatgtgaagtcacacatcaggggcttagcagccccgttacaaaatcctttaacaggggggagtgctgataagaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgggctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggatccgctctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacaggggtaccgtcgacgccgccaccatggagtggagctgggtgtttttgtttttcctgagtgtgaccactggagtccacagtgaggtccagttggtcgagtctggggggggactcatccagcctggtggcagtctgcgtctctcctgcgcagcttccggctttaccgtgtcctctaactacatgtcatgggttcgacaggctccagggaagggtctggagtgggtgtcaattatctatcccggagggtctactgagtatgcagacagcgttaaaggtcgattcacaattagccgcgataattcccggaacaccctgtatctgcaaatgaatagcttgagggccgaagacaccgctgtgtattactgcgcccgggagctcggctactacgggatggatgtgtggggtcagggaaccaccgtcaccgtctcctccgctagcaccaagggcccatctgtgtttccacttgctccttcttctaaaagcaccagcggtgggacagccgccctgggttgcctggtcaaagactacttccccgaacctgtcaccgtcagctggaactcaggagctctgacatccggggttcataccttccctgcagtgctgcaaagctcagggctgtacagtttgtctagcgtagtgaccgtgccttcttcctccctgggaacccagacctacatctgcaacgtgaatcataagccatctaatactaaagttgataagagagtggaacccaaaagttgtgataaaactcacacttgccctccttgtccagctcctgaagcagccggaggacccagtgtgtttctgttccctcccaagcccaaggacactctgatgattagccgtactcctgaagtcacttgtgtggttgtggatgttagccatgaggaccccgaagtgaagtttaactggtacgtggatggcgttgaggttcataatgctaaaaccaagccaagggaagaacagtacaatagcacctaccgggtcgtatcagtcctgactgtcttgcatcaagattggcttaacggcaaggagtacaagtgcaaggtaagcaataaggctctcccagctcccattgagaaaactatttcaaaggccaaaggtcaacctcgcgagcctcaggtgtacaccctgccaccttctcgcgaagaaatgacaaaaaaccaggttagcctgacctgtcttgttaagggattttacccatctgatatcgctgtagaatgggagtctaatgggcagccagagaataactataagacaactcctcccgtcctggactccgacggttctttttttctctactctaaacttacagtggacaaatcaagatggcagcagggaaatgtcttttcctgctccgttatgcacgaggccctgcataatcattacacccagaaaagcttgtctctgtcccctggcaaatgataagaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggg

SEQ ID NO: 37 - protein, 1270 aa, Severe acute respiratory syndrome coronavirus 2
  SITE (1)..(1270): SARS-CoV-2 Alpha variant S protein
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 1270 aa, Severe acute respiratory syndrome coronavirus 2
  SITE (1)..(1270): SARS-CoV-2 Beta variant S protein
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - protein, 1273 aa, Severe acute respiratory syndrome coronavirus 2
  SITE (1)..(1273): SARS-CoV-2 Gamma variant S protein
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - protein, 1271 aa, Severe acute respiratory syndrome coronavirus 2
  SITE (1)..(1271): SARS-CoV-2 Delta variant S protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 1271 aa, Severe acute respiratory syndrome coronavirus 2
  SITE (1)..(1271): SARS-CoV-2 Delta plus variant S protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 42 - protein, 1273 aa, Severe acute respiratory syndrome coronavirus 2
  SITE (1)..(1273): SARS-CoV-2 Kappa variant S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTDWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - DNA, 1738 bp, Artificial Sequence
  Synthetic Desmin enhancer/promoter long version
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacaggggtaccgtcgacgccgccacc

SEQ ID NO: 44 - DNA, 1265 bp, Artificial Sequence
  Synthetic Desmin enhancer/promoter short version
ccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagccactacgggtctaggctgcccatgtaaggaggcaaggcctggggacacccgagatgcctggttataattaacccagacatgtggctgcccccccccccccaacacctgctgcctgctaaaaataaccctgtccctggtggtctagaggaggggggcaagggcagggggaggcaccttcctcaggacatcgggtcctagagggaccttgctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatataaaaggtgagctcgtttagtgaaccgtcagtccgcctggagacctcgagccgagcggtcgtccgccgcctcctccgtgcgcccgccagcctcgcccgcgccgtcacctctagacacaggtaagtatcaaggttacaagacaggtttaaggaggccaatagaaactgggcttgtcgagacagagaagattcttgcgtttctgataggcacctattggtcttactgacatccactttgcctttctctccacaggggtaccgtcgacgccgccacc

SEQ ID NO: 45 - protein, 10 aa, Artificial Sequence
  Synthetic IgG1 upper hinge sequence
EPKSCDKTHT

SEQ ID NO: 46 - protein, 30 aa, Artificial Sequence
  Synthetic peptide linker
  SITE (6)..(30): “Gly Gly Gly Gly Ser” may be present or absent
GGGGSGGGGSGGGGSGGGGSGGGGSGGGGS

(19) United States
(12) Patent Application Publication
(10) Pub. No.: US 2023/0416343 A1
(43) Pub. Date: Dec. 28, 2023

(54) Neutralizing Antibodies that Bind the SARS-COV-2 S Protein

(71) Applicant: Sorrento Therapeutics, Inc., San Diego, CA (US)

(72) Inventors: Henry Hongjun Ji, Rancho Santa Fe, CA (US); Robert Allen, Corvallis, OR (US); Heyue Zhou, San Diego, CA (US); Yanliang Zhang, San Diego, CA (US); Yanwen Fu, San Diego, CA (US); Wenzhong Guo, San Diego, CA (US); Hui Xie, San Diego, CA (US); Xia Cao, San Diego, CA (US); Lucy Lu, San Diego, CA (US); Donghui Li, San Diego, CA (US); Reyna Lim, San Diego, CA (US); Arthur Ledesma, San Diego, CA (US); Daniel Lee, San Diego, CA (US)

(73) Assignee: Sorrento Therapeutics, Inc., San Diego, CA (US)

(21) Appl. No.: 18/040,617

(22) PCT Filed: Aug. 6, 2021
(86) PCT No.: PCT/US21/45010

§ 371 Date: Feb. 3, 2023

Related U.S. Application Data
(60) Provisional application No. 63/063,021, filed on Aug. 7, 2020.
(60) Provisional application No. 63/085,088, filed on Sep. 29, 2020.
(60) Provisional application No. 63/106,823, filed on Oct. 28, 2020.
(60) Provisional application No. 63/137,714, filed on Jan. 14, 2021.
(60) Provisional application No. 63/138,754, filed on Jan. 18, 2021.
(60) Provisional application No. 63/140,182, filed on Jan. 21, 2021.

(51) Int. Cl. C07K 16/10; A61P 31/14

(52) U.S. Cl. CPC C07K 16/1003; A61P 31/14; C07K 2317/565; C07K 2317/92; C07K 2317/52

(57) ABSTRACT

The present disclosure provides fully human antibodies that specifically bind the spike (S) protein of the SARS-CoV-2 coronavirus with high affinity, or antigen-binding proteins derived from such antibodies, and uses thereof. Included are anti-spike protein antibodies, antibody fragments, and single-chain antibodies, that are coronavirus neutralizing antibodies, as well as pharmaceutical compositions that include such antibodies and antibody fragments. Methods for using the anti-spike protein antibodies include methods of treating or preventing infection with a coronavirus, such as the SARS-CoV-2 coronavirus, by administering an antibody or antibody fragment as disclosed herein, including by intranasal delivery. Methods and compositions for treating or preventing coronavirus infection by administering a composition that includes a nucleic acid construct that encodes a neutralizing antibody are also provided.

### CROSS REFERENCE TO RELATED APPLICATIONS

This application claims the benefit of priority to U.S. provisional application No. 63/063,021, filed Aug. 7, 2020, to U.S. provisional application No. 63/085,088, filed Sep. 29, 2020, to U.S. provisional application No. 63/106,823, filed Oct. 28, 2020, to U.S. provisional application No. 63/137,714, filed Jan. 14, 2021, to U.S. provisional application No. 63/138,754, filed Jan. 18, 2021, and to U.S. provisional application No. 63/140,182, filed Jan. 21, 2021, the contents of each of which are incorporated herein by reference in their entireties.

### SEQUENCE LISTING

The present application is filed with a Sequence Listing in electronic format. The Sequence Listing is provided as a file entitled 01223-0085-OOPCT_ST25.txt and created on Aug. 4, 2021, which is 161,228 bytes in size. The information in the electronic format of the sequence listing is incorporated herein by reference in its entirety.

### TECHNICAL FIELD

The present disclosure provides antigen binding proteins that specifically bind the spike protein (S protein) of SARS-CoV-2 and nucleic acids that encode the antigen binding proteins, vectors comprising the nucleic acids, host cells harboring the vectors. The disclosure encompasses methods of treatment of a subject infected with, suspected of being infected with, or at risk of being affected with, a coronavirus, using the described antigen-binding proteins.

### BACKGROUND

The Severe Acute Respiratory Syndrome Coronavirus 2 (SARS-CoV-2, previously called 2019-nCoV), is the causative agent of the deadly Covid-19 pandemic. By the end of June 2020, over 10 million infections were reported worldwide, with over half a million deaths.

SARS-CoV-2 gains entry to human cells by using the angiotensin-converting enzyme 2 (ACE2) protein as a receptor. The spike (S) proteins of both SARS-CoV and SARS-CoV-2 are transmembrane glycoproteins that form homotrimers. Binding of ACE2 on host cells by the S protein leads to internalization of the virus.

The SARS-CoV-2 spike protein (S protein, NCBI Accession YP_009724390, isolate “Wuhan-Hu-1”) includes two regions or domains known as S1 (the N-terminus to amino acid 685) and S2 (amino acids 686 to 1273) that are cleaved into subunits by a cellular protease during the infection process. The S1 subunit, which mediates the interaction between the Spike (S) protein and ACE2, includes the “N-terminal domain” (NTD) which is followed by the receptor binding domain (RBD) at amino acids 331 to 524. The S2 subunit, which includes an extracellular domain, a transmembrane domain, and a cytoplasmic tail, mediates virus-host membrane fusion that results in entry of the virus into the host cell.

The appearance of variants of the original SARS-CoV-2 Washington State isolate (WA-1/2020), including the Alpha (“UK”) variant, the beta variant, the gamma variant, the delta and delta plus variants, and the kappa variant, has complicated efforts to prevent spread of the virus. There is an urgent need to find therapeutic and prophylactic agents that can be rapidly deployed to treat individuals with Covid-19 and halt the destructive spread of the SARS-CoV-2 pandemic.

### SUMMARY

To treat and prevent infection of individuals with SARS-CoV-2 and, potentially, other related coronaviruses, fully human neutralizing antibodies have been engineered that are able to inhibit binding of SARS-CoV-2 to target cells expressing the ACE2 protein. In some embodiments these antibodies are further engineered to include mutations of the Fc region, such as mutations to reduce antibody-dependent-enhancement (ADE) of infection.

Provided herein in a first aspect are antigen-binding proteins that specifically bind the spike (S) protein of the SARS-CoV-2 coronavirus (Genbank Accession QHD43416, or a variant S protein having at least 80% amino acid identity thereto), where the antigen-binding proteins comprise a heavy chain variable domain sequence having at least 95%, at least 96%, at least 97%, at least 98%, or at least 99% sequence identity to the amino acid sequence of SEQ ID NO:28 and a light chain variable region having at least 95%, at least 96%, at least 97%, at least 98%, or at least 99% sequence identity to the amino acid sequence of SEQ ID NO:29. In some examples an antigen-binding protein as provided herein that specifically binds the S protein of the SARS-CoV-2 coronavirus comprises a heavy chain variable domain having the sequence of SEQ ID NO:28 and a light chain variable domain having the sequence of SEQ ID NO:29.

In a related aspect are antigen-binding proteins are provided that specifically bind the S protein of SARS-CoV-2 (e.g., Genbank Accession QHD43416, or a variant S protein having at least 80% amino acid identity thereto) where the antigen-binding proteins comprise a heavy chain complementarity-determining region (CDR) 1 sequence having the amino acid sequence of SEQ ID NO:30, a heavy chain CDR2 sequence having the amino acid sequence of SEQ ID NO:31, and a heavy chain CDR3 sequence having the amino acid sequence of SEQ ID NO:32, and further comprise a light chain CDR1 sequence having the amino acid sequence of SEQ ID NO:33, a light chain CDR2 sequence having the amino acid sequence of SEQ ID NO:34, and a light chain CDR3 sequence having the amino acid sequence of SEQ ID NO:35. In various embodiments the antigen-binding proteins having the heavy chain CDR1 sequence of SEQ ID NO:30, the heavy chain CDR2 sequence of SEQ ID NO:31, the heavy chain CDR3 sequence of SEQ ID NO:32, the light chain CDR1 sequence of SEQ ID NO:33, the light chain CDR2 sequence of SEQ ID NO:34, and the light chain CDR3 sequence of SEQ ID NO:35 and have a heavy chain variable region comprising an amino acid sequence having at least 95%, at least 96%, at least 97%, at least 98%, or at least 99% sequence identity to the amino acid sequence of SEQ ID NO:28 and a light chain variable region comprising an amino acid sequence 95%, at least 96%, at least 97%, at least 98%, or at least 99% sequence identity to the amino acid sequence of SEQ ID NO:29.

An antigen-binding protein as provided herein can be or can be derived from an antibody, such as an IgG, IgA, IgD, IgE, or IgM antibody, that specifically binds the S protein of SARS-CoV-2. In various embodiments the anti-S protein antibody comprises or is derived from an IgG1, IgG2, IgG3, or IgG4 antibody. For example, the anti-S protein antibody or antigen-binding protein can comprise or be derived from an IgG1 or IgG4 antibody.

In further embodiments, an antigen-binding protein as provided herein comprising a heavy chain variable domain sequence having at least 95% amino acid sequence identity to SEQ ID NO:28 and a light chain variable region having at least 95% amino acid sequence identity to SEQ ID NO:29 and/or having the heavy chain CDR sequences of SEQ ID NOs:30, 31, and 32 and the light chain CDR sequences of SEQ ID NOs:33, 34, and 35 can be or comprise an antibody fragment, such as for example a Fab fragment, a Fab′ fragment, or a F(ab′)2 fragment. In additional embodiments an antigen-binding protein as provided herein having an amino acid sequence with at least 95% identity to SEQ ID NO:28 and an amino acid sequence with at least 95% identity to SEQ ID NO:29 and/or having the heavy chain CDR sequences of SEQ ID NOs:30, 31, and 32 and the light chain CDR sequences of SEQ ID NOs:33, 34, and 35 can be or comprise a single chain antibody (e.g., an scFv or scFab).

In some embodiments, the antigen-binding protein provided herein is a fully human antibody or a fully human antibody fragment, for example, a fully human IgG1, IgG2, IgG3, IgG4, IgA, IgD, IgE, or IgM, a fully human single chain antibody, a fully human Fab fragment, or is a single chain antibody, or is an antigen binding protein derived from or comprising any of these.

In some embodiments the antibody is an IgG antibody having one or more mutations in the Fc region, for example one or more mutations that decreases antibody dependent enhancement (ADE) and/or one or more mutations that increases antibody half-life. In some embodiments the antibody has one or more mutations in the Fc region that reduce ADE selected from L234A; L235A or L235E; N297A, N297Q, or N297D; and P329A or P329G. For example, the anti-S antibody can include the mutations L234A and L235A (referred to as a LALA mutant). Alternatively or in addition, the antibody may have one or more mutations in the Fc region that increase the half-life of the antibody in serum, for example, the antibody may have one or more mutations selected from M252Y; S254T; T256D or T256E; T307Q or T307W; M428L; and N434S. For example, the anti-S antibody can include the mutations M252Y; S254T; and T256E (referred to as a YTE mutant).

In some embodiments, the antigen-binding protein that specifically binds the S protein of SARS-CoV-2, which can be, as nonlimiting examples, an IgG, a Fab fragment, or a single chain antibody, or can be an antigen-binding protein comprising or derived from any thereof, specifically binds a coronavirus spike protein (e.g., a spike protein comprising SEQ ID NO:1 or SEQ ID NO:2, or a spike protein of a coronavirus comprising a sequence having at least 95%, 96%, 97%, 98%, or 99% identity to SEQ ID NO:1 or SEQ ID NO:2) with a K_{d} of less than 200 nM, less than 100 nM, less than 50 nM, less than 10 nM, less than 1 nM, less than 0.1 nM, or less than 0.01 nM. For example, the antigen-binding protein can bind the S protein of a coronavirus (e.g., the S protein of SARS-CoV-2) with a K_{d} of between of between about 200 nM and about 0.01 nM, or between 100 nM and 0.1 nM, between 100 nM and 1 nM, or between 10 nM and 0.1 nM. In some embodiments, the antibody is the S1D7270 antibody having a heavy chain variable sequence of SEQ ID NO:28 and a light chain variable sequence of SEQ ID NO:29, and optionally comprising one or more mutations in the Fc region, having a K_{d} for binding the S protein of SARS-CoV-2 of between about 10 nM and about 0.1 nM or between about 5 nM and about 0.5 nM.

In some embodiments, an antigen-binding protein provided herein, which can in various embodiments be an antibody, such as a fully human antibody, and may be, as nonlimiting examples, an IgG, a Fab fragment, or a single chain antibody, or can be an antigen-binding protein derived from any thereof, specifically binds the S1 subunit of a coronavirus S protein (e.g., SEQ ID NO:4 or an S1 subunit of a spike protein of a coronavirus comprising a sequence having at least 95%, 96%, 97%, 98%, or 99% identity to SEQ ID NO:4) with a K_{d} of less than 200 nM, less than 100 nM, less than 50 nM, less than 10 nM, less than 1 nM, less than 0.1 nM, or less than 0.01 nM. For example, the antigen-binding protein can bind the S1 subunit of a coronavirus (e.g., the S1 subunit of SARS-CoV-2) with a K_{d} of between of between about 200 nM and about 0.01 nM, or between 100 nM and 0.1 nM, between 100 nM and 1 nM, or between about 10 nM and about 0.1 nM. In one embodiment, the antibody is the fully human S1D7270 antibody or antibody fragment, or an antigen-binding protein derived therefrom, having a variable heavy chain sequence of SEQ ID NO:28 and a variable light chain sequence of SEQ ID NO:29, and optionally comprising one or more mutations in the Fc region, that binds the S1 subunit of the S protein of SARS-CoV-2 with a K_{d} of between about 10 nM and about 0.1 nM or between about 5 nM and about 0.5 nM.

In some embodiments, an antigen-binding protein provided herein, which can be in various embodiments be an antibody, such as a fully human antibody, and may be, as nonlimiting examples, an IgG, a Fab fragment, or a single chain antibody, or can be an antigen-binding protein derived from any thereof, specifically binds the receptor binding domain (RBD) of a coronavirus S protein (e.g., SEQ ID NO:5 or an RBD of a spike protein of a coronavirus comprising an amino acid sequence having at least 95%, 96%, 97%, 98%, or 99% identity to SEQ ID NO:5) with a K_{d} of less than 100 nM, less than 50 nM, less than 10 nM, less than 1 nM, less than 0.1 nM, or less than 0.01 nM. For example, the antigen-binding protein can bind the RBD of the S protein of a coronavirus (e.g., the RBD the SARS-CoV-2 S protein) with a K_{d} of between of between about 200 nM and about 0.01 nM, between 100 nM and 0.1 nM, or between 10 nM and 0.1 nM. In some embodiments, the antigen binding protein is the fully human S1D7270 antibody having a variable heavy chain sequence of SEQ ID NO:28 and a variable light chain sequence of SEQ ID NO:29, and optionally comprising one or more mutations in the Fc region, that binds the RBD of the S protein of SARS-CoV-2 with a K_{d} of between about 10 nM and about 0.1 nM or between about 5 nM and about 0.5 nM. For example, the antigen binding protein may be the fully human S1D7270 antibody disclosed herein having a variable heavy chain sequence of SEQ ID NO:28 and a variable light chain sequence of SEQ ID NO:29, and optionally having L234A and L235A mutations in the Fc region, where the antibody binds the RBD of the S protein of SARS-CoV-2 with a K_{d} of between about 10 nM and about 0.1 nM, or between about 5 nM and about 0.2 nM, or between 5 nM and 1 nM as measured by SPR.

In various embodiments, the antigen-binding proteins described herein block binding between the S protein of a coronavirus (such as HCoV-NL63, SARS-CoV, or SARS-CoV-2) and the ACE2 protein, for example, block binding of the ectodomain of the human ACE2 protein (hACE2) by the S protein of a coronavirus. In various embodiments, the antigen-binding proteins described herein block binding between the S protein of SARS-CoV-2 and the human ACE2 protein with an IC₅₀ of between about 0.01 μg/ml and about 100 μg/ml, between about 0.05 μg/ml and about 50 μg/ml, between about 0.1 μg/ml and about 10 μg/ml, or between about 0.1 μg/ml and about 5 μg/ml. In some embodiments, the antigen binding proteins described herein block binding between the S1 domain or subunit of SARS-CoV-2 and the human ACE2 protein with an IC₅₀ of between about 0.01 μg/ml and about 100 μg/ml, between about 0.05 μg/ml and about 50 μg/ml, between about 0.1 μg/ml and about 10 μg/ml, or between about 0.1 μg/ml and about 5 μg/ml. For example, an antigen-binding protein as disclosed herein can block the binding of the S1 subunit of SARS-CoV-2 and the human ACE2 protein with an IC₅₀ of less than 100 nM, less than 50 nM, less than 10 nM, less than 5 nM, or less than 1 nM.

For example, an antigen-binding protein as disclosed herein that includes an amino acid sequence having at least 95% identity to SEQ ID NO:28 and an amino acid sequence having at least 95% identity to SEQ ID NO:29 can in some embodiments block the binding of the S1 subunit of SARS-CoV-2 (e.g., SEQ ID NO:4) to the ACE2 polypeptide (or the ACE2 ectodomain, e.g., SEQ ID NO:23 or SEQ ID NO:24) with an IC₅₀ of less than 100 nM, less than 50 nM, less than 10 nM, less than 5 nM, or less than 1 nM, for example, with an IC₅₀ of between about 100 nM and about 0.1 nM or between about 50 nM and about 0.5 nM, or between about 10 nM and about 0.1 nM, and in some embodiments between about 5 nM and about o0.5 nM. In some embodiments the antigen binding protein is the fully human S1D7270 antibody having a variable heavy chain sequence of SEQ ID NO:28 and a variable light chain sequence of SEQ ID NO:29, and optionally comprising one or more mutations in the Fc region, that binds the S1 subunit of the S protein of SARS-CoV-2 and can block binding of the SARS-CoV-2 S1 subunit to the ACE2 protein or the ectodomain thereof with an IC₅₀ of between about 10 nM and about 0.1 nM, for example between about 5 nM and about 0.5 nM, or between about 2 nM and about 1 nM.

In various embodiments provided herein, an antigen-binding protein as disclosed herein, which can be or comprise, as nonlimiting examples, an IgG1, IgG2, IgG3, or IgG4, a Fab fragment or a single chain antibody, or can be an antigen binding protein derived from or comprising any of these, is a neutralizing antigen binding protein that is able to inhibit binding to a target cell by a coronavirus such as HCoV-NL63, SARS-CoV, or SARS-CoV-2. For example, in various embodiments the antigen-binding protein, when included in a mixture that includes coronavirus and target cells expressing the ACE2 receptor, can reduce binding of the coronavirus to cells expressing the ACE2 receptor with an IC₅₀ of between about 0.001 μg/ml and about 200 μg/ml, or between about 0.01 μg/ml and about 100 μg/ml, or between about 0.01 μg/ml and about 50 μg/ml, or between about 0.01 μg/ml and about 10 μg/ml, or between about 0.01 μg/ml and about 5 μg/ml, or between about 0.01 μg/ml and about 1 μg/ml, or between about 0.1 μg/ml and about 100 μg/ml, or between about 0.1 μg/ml and about 50 μg/ml. In some embodiments an antigen-binding protein can reduce binding of the coronavirus to a cell expressing the ACE2 receptor with an IC₅₀ of between about 1 μg/ml and about 50 μg/ml, or between about 1 μg/ml and about 10 μg/ml, or between about 1 μg/ml and about 5 μg/ml. In additional embodiments an antigen-binding protein can reduce binding of the coronavirus to a cell expressing the ACE2 receptor with an IC₅₀ of between about 0.1 μg/ml and about 10 μg/ml, or between about 0.1 μg/ml and about 5 μg/ml, or between about 0.1 μg/ml and about 1 μg/ml.

Any of the antibodies or antigen binding proteins disclosed herein may be isolated. Any of the antibodies disclosed herein may be humanized or fully human antibodies.

In a further aspect provided herein are pharmaceutical compositions that include a neutralizing antigen-binding protein as disclosed herein that specifically binds the S protein of a coronavirus, such as the S protein of SARS-CoV-2, and a pharmaceutically carrier. A neutralizing antigen-binding protein as disclosed herein and considered for use in the pharmaceutical compositions and methods provided herein comprises a heavy chain variable domain sequence having at least 95%, at least 96%, at least 97%, at least 98%, or at least 99% sequence identity to the amino acid sequence of SEQ ID NO:28 and a light chain variable region having at least 95%, at least 96%, at least 97%, at least 98%, or at least 99% sequence identity to the amino acid sequence of SEQ ID NO:29, where the antigen-binding protein specifically binds the S protein of SARS-CoV-2 and has neutralizing activity with respect to at least one coronavirus, e.g., SARS-CoV-2. In various embodiments a neutralizing antibody as disclosed herein specifically binds the S protein of SARS-CoV-2 and includes heavy chain CDR1, CDR2, and CDR3 sequences of SEQ ID NO:30, SEQ ID NO:31, and SEQ ID NO:32, respectively, and further includes light chain CDR1, CDR2, and CDR3 sequences of SEQ ID NO:33, SEQ ID NO:34, and SEQ ID NO:35, respectively.

The pharmaceutical composition can be formulated for intramuscular or subcutaneous injection, or for intravenous, oral, nasal, or pulmonary delivery, as nonlimiting examples. The pharmaceutical composition can optionally be formulated as a liquid or solid, depending on the mode of delivery and/or storage and packaging considerations, and can optionally be formulated and packaged in single doses. In some embodiments, the pharmaceutical can be formulated as a liquid composition for intravenous infusion. In further embodiments, the pharmaceutical can be formulated as a liquid composition for nasal delivery, e.g., for deposition in nasal passages, and may be packaged as a liquid, for example, in a spray bottle or a bottle that allows for depositing the liquid formulation in the nostrils by squeezing or by means of a dropper or syringe that may be provided with the liquid formulation.

Also provided herein in another aspect is a method of treating a subject infected or suspected of being infected with a coronavirus such as SARS-CoV or SARS-Cov-2. The method includes administering an effective amount of an antigen-binding protein as disclosed herein, e.g., a neutralizing antibody as disclosed herein, for example in a pharmaceutical composition as disclosed herein, to the subject. Administration can be, as nonlimiting examples, by intramuscular or subcutaneous injection, by intravenous delivery, by oral delivery, by nasal delivery (e.g., topical nasal delivery), or by pulmonary delivery, for example by inhalation. Further included is a method of preventing infection with a coronavirus such as HCoV-NL63, SARS-CoV, or SARS-Cov-2. The method includes administering an effective amount of the antibody disclosed herein, for example in a pharmaceutical composition as disclosed herein, to the subject. Administration can be, as nonlimiting examples, by intramuscular or subcutaneous injection, or by intravenous, oral, nasal, or pulmonary delivery, such as by inhalation

In various embodiments the disclosure provides methods of treating infection with a coronavirus such as HCoV-NL63, SARS-CoV, or SARS-CoV-2 by nasal administration of a neutralizing antibody. Using the methods provided herein, a pharmaceutical composition that includes at least one neutralizing antibody can be deposited in the nasal passages of a subject within one, two, three, four, five, six, seven or more days of exposure or suspected exposure of the subject to a coronavirus or at any time after diagnosis of infection with coronavirus. For example, using the methods provided herein, a pharmaceutical composition that includes at least one neutralizing antibody can be deposited in the nasal passages of a subject exhibiting symptoms of infection with a coronavirus or having a confirmed infection with a coronavirus. In various embodiments provided herein, the pharmaceutical composition is deposited in the nasal passages without the use of an inhalation device such as a nebulizer, dry powder inhaler, or metered-dose inhaler. The pharmaceutical composition can include more than one therapeutic compound, and can include, for example, more than one neutralizing antibody.

In a further aspect, a subject having a coronavirus infection, exhibiting symptoms of a coronavirus infection, or having had exposure or suspected exposure to a coronavirus, can be treated with a nasal composition as provided herein that includes a neutralizing antibody that binds a coronavirus and with an intravenously delivered neutralizing antibody that binds a coronavirus. In various embodiments the neutralizing antibody in the nasal pharmaceutical composition, the intravenous pharmaceutical composition, or both, can be any described herein, and one or both of the pharmaceutical compositions can include more than one neutralizing antibody. In some examples, a subject is treated with an intranasal composition that includes a neutralizing antibody and an intravenous composition that includes a neutralizing antibody on the same day. In some examples, a subject is treated with an intranasal composition that includes a neutralizing antibody and an intravenous composition that includes a neutralizing antibody on the different days. The subject can receive a single dose or multiple doses of an intranasal neutralizing antibody composition and an intravenous neutralizing composition. In various embodiments, one or both of a neutralizing antibody in the intranasal composition and a neutralizing antibody in the intravenous composition are a neutralizing antibody as provided herein, such as, for example, a neutralizing antibody having a heavy chain variable domain sequence having at least 95%, at least 96%, at least 97%, at least 98%, or at least 99% sequence identity to the amino acid sequence of SEQ ID NO:28 and a light chain variable region having at least 95%, at least 96%, at least 97%, at least 98%, or at least 99% sequence identity to the amino acid sequence of SEQ ID NO:29. In various embodiments a neutralizing antibody as disclosed herein specifically binds the S protein of SARS-CoV-2 and includes heavy chain CDR1, CDR2, and CDR3 sequences of SEQ ID NO:30, SEQ ID NO:31, and SEQ ID NO:32, respectively, and further includes light chain CDR1, CDR2, and CDR3 sequences of SEQ ID NO:33, SEQ ID NO:34, and SEQ ID NO:35, respectively.

Yet another aspect are methods of detecting a coronavirus using an antigen-binding protein that specifically binds the S protein of a coronavirus as disclosed herein. The methods include detecting the presence of a coronavirus, or a protein of a coronavirus, e.g., an S protein or S1 subunit of a coronavirus, in a sample, comprising: (a) contacting the sample with an antigen-binding protein as disclosed herein under conditions suitable to form an antibody-antigen complex; and (b) detecting the presence of the antibody-antigen complex to detect the presence of a coronavirus or protein thereof. In some embodiments, this method can be used to detect the presence of a coronavirus in a sample from a subject and thereby diagnose a subject suspected of having a coronavirus infection. In various embodiments the sample from the subject comprises phlegm, mucous, saliva, blood, pleural fluid, cheek scaping, tissue biopsy, or semen. In some embodiments, the antigen-binding protein that specifically binds the S protein, which can be an antibody or antibody fragment that specifically binds the S protein, can be labeled for direct or indirect detection of an antigen-antibody complex, where the label can comprise a radionuclide, fluorophore, enzyme, enzyme substrate, enzyme cofactor, enzyme inhibitor, or ligand (e.g., biotin, a hapten). In various embodiments, the presence of the antibody-antigen complex can be detected using any detection mode including detection of radioactivity, detection of fluorescence, detection of luminescence, or colorimetric, antigenic, or enzymatic detection, or detection of a magnetic or electrodense (e.g., gold) bead, and may optionally use binding moieties such as but not limited to biotin, streptavidin, or protein A.

Also included herein are nucleic acid molecules encoding an antigen-binding protein comprising a heavy chain variable domain sequence having at least 95% amino acid sequence identity to SEQ ID NO:28 and nucleic acid molecules encoding a light chain variable region having at least 95% amino acid sequence identity to SEQ ID NO:29. A nucleic acid molecule as provided herein can encode one or both of a heavy chain variable domain sequence having at least 95% amino acid sequence identity to SEQ ID NO:28 and a light chain variable region having at least 95% amino acid sequence identity to SEQ ID NO:29, and can be an expression vector that includes a promoter or promoters operably linked to the antigen-binding protein encoding sequence(s).

Also included herein are pharmaceutical compositions that include nucleic acid molecules that may be administered to a subject, such as a human subject for treatment or prevention of a coronavirus infection. A nucleic acid molecule that encodes a neutralizing antigen binding protein as provided herein can be an RNA molecule or a DNA molecule and can include one or more non-naturally occurring linkages (e.g., backbone linkages) or nucleobases.

A nucleic acid molecule provided in a pharmaceutical composition can be, for example, DNA encoding a neutralizing antigen-binding protein and can be a plasmid, where the plasmid can encode an antigen-binding protein comprising a heavy chain variable domain sequence having at least 95% amino acid sequence identity to SEQ ID NO:28 and a light chain variable region having at least 95% amino acid sequence identity to SEQ ID NO:29. A nucleic acid molecule as provided herein can encode one or both of a heavy chain variable domain sequence having at least 95% amino acid sequence identity to SEQ ID NO:28 and a light chain variable region having at least 95% amino acid sequence identity to SEQ ID NO:29, and can be an expression vector that includes a promoter or promoters operably linked to the antigen-binding protein encoding sequence(s). A pharmaceutical composition can include one or more nucleic acid molecules, for example, can include a nucleic acid molecule that encodes a heavy chain of an antibody and a second nucleic molecule that encodes a light chain of an antibody, or a pharmaceutical composition can include a single nucleic acid construct that includes two open reading frames or genes, each operably linked to its own promoter, for example, encoding a light chain of an antibody and a heavy chain of an antibody. In some embodiments the composition is formulated for intramuscular injection, and a first gene encoding a light chain of an antibody and a second gene encoding a heavy chain of the antibody are each independently linked to a promoter active in muscle cells. The heavy and light chain genes can be on the same or different nucleic acid molecules. The nucleic acid molecule(s) can be a plasmid, for example, a nanoplasmid having fewer than 500 base pairs of sequence of a bacterial plasmid, three or fewer CpG sequences, and/or can lack an antibiotic resistance marker such as any disclosed in U.S. Pat. Nos. 9,550,998; 10,047,365; or 10,844,388, all of which are incorporated herein by reference in their entireties.

A pharmaceutical composition can include compounds that enhance delivery of nucleic acid molecules into cells, such as for example amphiphilic block copolymers, for example, linear and/or X-shaped copolymers, and can include one or more poloxamers or poloxamines, or of any of an ethylene oxide/propylene oxide copolymer, Synperonics®, Pluronics®, Kolliphor®, poloxamer 181, poloxamer 188, or poloxamer 407, poloxamines, Tetronics®, T/908, or T/1301. The pharmaceutical composition can further include any of alginate, a cationic lipid, or a PEG polymer or copolymer, e.g., DSPE-PEG, as nonlimiting examples, and can be formulated for injection and can include a buffer such as PBS, TBS, Ringer's, or Tyrode's.

Provided herein are methods of treatment and methods of prophylaxis that include administering to a subject a composition as described that includes at least one nucleic acid construct that encodes a neutralizing antibody that binds a coronavirus. The subject can be a human subject, for example, a human subject that has a coronavirus infection, is suspected of having a coronavirus infection, or is at risk of becoming infected with a coronavirus. The subject can also be a non-human animal. The administering can be by injection, for example, intramuscular injection. Single or multiple doses, including multiple doses over weeks or months, can be administered. The amount of DNA (e.g., plasmid or plasmids encoding a neutralizing antibody) to be delivered can be determined for example, at least in part by experiments on non-human animals.

Further included are transgenic cells engineered to express an antibody comprising a heavy chain variable domain sequence having at least 95% amino acid sequence identity to SEQ ID NO:28 and a light chain variable region having at least 95% amino acid sequence identity to SEQ ID NO:29. The cells can include one or more nucleic acid molecules that encodes a heavy chain variable domain and/or light chain variable domain as disclosed herein. The cells can be prokaryotic or eukaryotic. In some embodiments the transgenic cells are mammalian cells, such as cells of a mammalian cell line.

### BRIEF DESCRIPTION OF THE DRAWINGS

FIG. 1 is a bar graph showing the results of a blocking ELISA in which twenty-three IgG variants of parental antibody S1D2 were assayed alongside S1D2 for the ability to inhibit binding of the SARS-CoV-2 S1 protein to the ACE2 protein.

FIG. 2 provides a sensorgram for S1D2 variant antibody S1D7270 binding to the RBD of the SARS-CoV-2 S protein and a table of the binding parameters.

FIG. 3 provides IC₅₀ curves for the inhibition of binding of the SARS-CoV-2 S1 protein to the ACE2 protein by the S1D2 antibody and antibody variants of S1D2 based on ELISAs.

FIG. 4 provides EC50 curves for binding of the S1D2_{LALA} (STI-1499) antibody (lower curves of each graph) and S1D7270_{LALA} (STI-2020) antibody (upper curves of each graph) to the S protein (left graph) or D614G mutant S protein (right graph) expressed on the cell surface.

FIG. 5 provides a graph showing the inhibition of the cytopathic effect (CPE) of the SARS-CoV-2 WA isolate and the SARS-CoV-2 2020001 isolate by the S1D7270_{LALA} antibody and the S1D2_{LALA} antibody.

FIG. 6A provides graphs of the % change in individual weights over time of hamsters infected with SARS-CoV-2 and treated intravenously with the S1D7270_{LALA} (STI-2020) antibody at various doses and an isotype control. FIG. 6B is a graph in which the curves represent the averages of the % weight change each treatment group per day. FIG. 6C is a graph depicting the viral titer from lungs harvested from animals on Day 5 of the viral challenge study. Depicted are dots representing individual titers of tissue from animals treated with isotype control antibody, 500 μg of S1D7270_{LALA} (STI-2020) antibody, 300 μg S1D7270_{LALA} (STI-2020) antibody, and 100 μg S1D7270_{LALA} (STI-2020) antibody.

FIG. 7A-D provides graphs of the concentrations of neutralizing antibody STI-2020 found in the organs of mice treated with the antibody. (FIG. 7A) Concentration of STI-2020 in serum and lung lavage collected from female CD-1 mice administered STI-2020 intravenously (IV) at doses of 0.5 mg/kg (second column of each group), 0.05 mg/kg (third column of each group), or 0.005 mg/kg (fourth column of each group) at 24 hours post-administration as compared to samples collected from untreated mice (first column of each group). (FIG. 7B) Concentration of STI-2020 in lysates of collected spleens, lungs, small intestines, and large intestines from animals dosed intravenously at the same dose levels detailed in panel A. (FIG. 7C) Concentration of STI-2020 in serum and lung lavage collected following administration of STI-2020 intranasally (IN) at doses of 2.5 mg/kg (second column of each group), 0.5 mg/kg (third column of each group), 0.05 mg/kg (fourth column of each group), and 0.005 mg/kg (fifth column of each group) as compared to samples from untreated mice (first column of each group). (FIG. 7D) Concentration of STI-2020 in lysates of collected spleens, lungs, small intestines, and large intestines from animals dosed intranasally at the same dose levels detailed in panel C. Values represent mean±SEM (n=3 animals no treatment group, n=5 in treatment groups). Significant differences are denoted by *, P<0.05; **, P<0.01; ***, P<0.001, ****, P<0.0001.

FIG. 8A-C provides graphs of the concentrations of neutralizing antibody STI-2020 found in the lung and serum of mice treated intranasally with 5 mg/kg STI-2020 over time after antibody administration. (FIG. 8A) Concentration of STI-2020 in lung tissue collected from female CD-1 mice administered STI-2020 intranasally (IN) at a dose of 5 mg/kg. Samples from treated mice were collected at the indicated times post-administration and STI-2020 antibody concentrations were quantified by ELISA and compared to samples collected from untreated mice. (FIG. 8B) Concentration of STI-2020 in serum isolated from female CD-1 mice administered STI-2020 intranasally (IN) at a dose of 5 mg/kg. Serum samples were collected from treated mice and compared to samples from untreated mice. (FIG. 8C) Overlay of STI-2020 concentrations in lung tissue vs. serum following IN administration of a 5 mg/kg dose. Values represent mean SD (n=3 animals no treatment group, n=6 per time point in treatment groups).

FIG. 9 is a table providing pharmacokinetic (PK) parameters of STI-2020 in mice after intranasal dosing.

FIG. 10A-F provides graphs of % daily weight changes of animals administered intravenously (FIGS. 10A, B, and C) or intranasally (FIGS. 10D, E, and F) with neutralizing antibody STI-2020 twelve hours after inoculation wit SARS-CoV-2. Female hamsters were inoculated with SARS-CoV-2 WA-1 isolate on day 0. Twelve hours post-infection, animals (n=5 per group) were administered a single intravenous dose of Control IgG (500 mg, red circles) or STI-2020 (500 mg, blue squares). Daily weight changes from day 0 to day 10 were recorded and (FIG. 10B) plotted for each individual animal. (FIG. 10C) Average % daily weight change ±standard deviation was plotted for each group. (FIG. 10D) Female hamsters were infected as described in FIG. 10A. Twelve hours post-infection, a single dose of 500 mg STI-2020 or Control IgG was administered intranasally (IN) and daily weight changes were recorded and (FIG. 10E) plotted for each individual animal. (FIG. 10F) Average % daily weight change ±standard deviation was plotted for each group. Days on which there was a significant difference in average % weight change between STI-2020 500 mg-treated animals (blue squares) and Control IgG 500 mg-treated animals (red circles) are denoted by *, P<0.05; **, P<0.01.

FIG. 11 provides data from flow cytometry assays of binding of the S1D7270 antibody to surface expressed spike protein derived from emerging pandemic virus isolates. EC50s are provided in the table.

FIG. 12 shows luciferase expression in muscle two days after intramuscular injection of 10 μg of a DNA vector encoding luciferase. Fluorescence was measured using an in vivo imaging system (IVIS).

FIG. 13 provides serum levels of S1D7270 antibody following intramuscular injection into mice of nanoplasmid encoding the heavy and light chains of neutralizing antibody S1D7270. 50 μg of plasmid was administered to the gastrocnemius muscle and human IgG was detected by ELISA.

FIG. 14A-B is a graph providing the human IgG concentrations in plasma collected from individual Tg32 mice treated with a DNA plasmid encoding STI-2020 formulated with block polymer ICA 614. FIG. 14A shows the antibody concentration of plasma isolated of individual mice administered 100 μg of DNA on days 7, 14, 21, and 28 post-treatment, with the average values of the sample provided above the sample points. FIG. 14B shows the antibody concentration of plasma isolated of individual mice administered 100 μg of DNA on days 7, 14, 21, and 28 post-treatment, with the average values of the sample provided above the sample points. Error bars represent the standard deviations of the means.

FIG. 15A provides the curve for the percentage of plaque reduction with increasing expressed antibody concentration in the PRNT assay using pooled plasma derived from Tg32 mice 14 days after being treated with ICA614-formulated S1D7270_{LALA} (STI-2020)-encoding plasmid pNP-Muscle-S1D7270/HCLC. FIG. 15B provides the curve for the percentage of plaque reduction with increasing expressed antibody concentration in the PRNT assay using pooled plasma derived from Tg32 mice 28 days after being treated with ICA614-formulated S1D7270_{LALA} (STI-2020)-encoding plasmid pNP-Muscle-S1D7270/HCLC.

FIG. 16A provides the amounts of human IgG detected in quadriceps muscle tissue of mice sacrificed 28 days after injection with either 100 μg or 250 μg ICA614-formulated S1D7270_{LALA} (STI-2020)-encoding plasmid pNP-Muscle-S1D7270/HCLC. RQ, right quadricep; LQ, left quadricep. FIG. 16B provides the data as averages values from pooled right and left quadriceps samples.

FIG. 17A provides the EC50 curve for binding of the S1D7270_{LALA} (STI-2020) antibody to the S protein of the SARS-CoV-2 delta (B1.617.2) variant (including mutations T19R, T95I, G142D, del157-158, A222V, L452R, T478K, D614G, P681R, D950N). FIG. 17B provides the EC50 curve for binding of the S1D7270_{LALA} (STI-2020) antibody to the S protein of the SARS-CoV-2 delta (B1.617.2.1) variant (including mutations T19R, T95I, G142D, del157-158, A222V, K417N, L452R, T478K, D614G, P681R, and D950N). The EC50 for binding of STI-2020 to the delta spike protein on cells was found to be 0.1057 μg/ml and the EC50 for binding of STI-2020 to the delta plus spike protein on cells was found to be 0.1868 μg/ml in this assay.

### DESCRIPTION

Headings provided herein are solely for the convenience of the reader and do not limit the various aspects of the disclosure, which aspects can be understood by reference to the specification as a whole.

The disclosures of all publications, patents, and patent applications cited herein are hereby incorporated by reference in their entireties into this application.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation and delivery, and treatment of patients.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Where ranges are included in the description, the range includes the recited boundary values.

The terms “peptide”, “polypeptide” and “protein” and other related terms used herein are used interchangeably and refer to a polymer of amino acids that is not limited to any particular length. Polypeptides may comprise natural and non-natural amino acids. Polypeptides include recombinant and chemically-synthesized polypeptides. Polypeptides include precursor molecules and mature (e.g., processed) molecules. Precursor molecules include those that have not yet been subjected to cleavage, for example cleavage of a secretory signal peptide or by enzymatic or non-enzymatic cleavage at certain amino acid residue(s). Polypeptides include mature molecules that have undergone cleavage. These terms encompass native proteins, recombinant proteins, and artificial proteins, protein fragments and polypeptide analogs (such as muteins, variants, chimeric proteins and fusion proteins) of a protein sequence as well as post-translationally, or otherwise covalently or non-covalently, modified proteins. Polypeptides that bind the S protein of a coronavirus and that are produced using recombinant procedures are described herein.

The terms “nucleic acid”, “nucleic acid molecule”, “polynucleotide” and “oligonucleotide” and other related terms used herein are used interchangeably and refer to polymers of nucleotides that are not limited to any particular length. Nucleic acids include recombinant and chemically-synthesized forms. Nucleic acids include DNA molecules (e.g., cDNA or genomic DNA, expression constructs, DNA fragments, etc.), RNA molecules (e.g., mRNA), analogs of the DNA or RNA generated using nucleotide analogs (e.g., peptide nucleic acids and non-naturally occurring nucleotide analogs), and hybrids thereof, as well as peptide nucleic acids, locked nucleic acids, and other synthetic nucleic acid analogs and hybrids thereof. A nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In some embodiments, nucleic acids comprise one type of polynucleotides or a mixture of two or more different types of polynucleotides. Nucleic acids encoding anti-S protein antibodies or antigen-binding portions thereof are described herein.

The term “recover” or “recovery” or “recovering”, and other related terms, refer to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence (e.g., leader peptide sequence) which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or protein G (cell wall components from Staphylococcus aureus).

The term “isolated” refers to a protein (e.g., an antibody or an antigen binding portion thereof) or polynucleotide that is substantially free of other cellular material. The term isolated also refers in some embodiments to protein or polynucleotides that are substantially free of other molecules of the same species, for example other proteins or polynucleotides having different amino acid or nucleotide sequences, respectively. The purity or homogeneity of the desired molecule can be assayed using techniques well known in the art, including low resolution methods such as gel electrophoresis and high resolution methods such as HPLC or mass spectrometry. In various embodiments any of the anti-S antibodies disclosed herein (e.g., S1D7270 or STI-2020 antibodies or antigen binding proteins derived therefrom) are isolated.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “leader sequence” or “leader peptide” or “[peptide] signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length and is cleaved from the polypeptide upon secretion of the mature polypeptide or insertion of the mature polypeptide into the membrane. Thus, proteins provided herein such as membrane proteins and antibodies having signal peptides that are identified by their precursor sequences that include a signal peptide sequence are also intended to encompass the mature forms of the polypeptides lacking the signal peptide, and proteins provided herein such as membrane proteins and antibodies having signal peptides that are identified by their mature polypeptide sequences that lack a signal peptide sequence are also intended to encompass forms of the polypeptides that include a signal peptide, whether native to the protein or derived from another secreted or membrane-inserted protein. In one embodiment, a leader sequence includes signal sequences comprising CD8a, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO:17).

An “antigen-binding protein” and related terms used herein refer to a protein comprising a portion that binds to an antigen and, optionally, a scaffold or framework portion that allows the antigen binding portion to adopt a conformation that promotes binding of the antigen-binding protein to the antigen. Examples of antigen-binding proteins include antibodies, antibody fragments (e.g., an antigen binding portion of an antibody), antibody derivatives, and antibody analogs. As used herein an “antigen-binding protein derived from [a referenced] antibody” is an antigen-binding protein that includes the variable light chain sequence and variable heavy chain sequence of the referenced antibody. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind the spike protein of SARS-CoV-2 are described herein.

An antigen binding protein can have, in some examples, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of three hypervariable regions, also called complementarity determining regions or CDRs, joined by relatively conserved framework regions (FR). From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (e.g., “Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 J. Mol. Biol. 273:927-948; Contact (Maccallum et al., 1996 J. Mol. Biol. 262:732-745, and Aho (Honegger and Pluckthun 2001 J. Mol. Biol. 309:657-670.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)2, Fv, single domain antibodies (dAbs), and complementarity determining region (CDR) fragments, single-chain antibodies (scFv), chimeric antibodies, diabodies, triabodies, tetrabodies, nanobodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies (e.g., single chain Fab antibodies (scFabs), e.g., Hust et al. (2007) BMC Biotechnology 7:14), single chain variable fragment antibodies (scFvs), camelized antibodies, affibodies, disulfide-linked Fvs (sdFv), anti-idiotypic antibodies (anti-Id), minibodies. Antibodies include monoclonal and polyclonal antibody populations.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Monoclonal antibodies include monoclonal antibodies produced using hybridoma methods that provide a cell line producing a population of identical antibody molecules, and also include chimeric, hybrid, and recombinant antibodies produced by cloning methods such that a cell transfected with the construct or constructs that include the antibody-encoding sequences and the progeny of the transfected cell produce a population of antibody molecules directed against a single antigenic site. For example, variable regions of an antibody (variable heavy chain and light chain regions or variable heavy and light chain CDRs) may be cloned into an antibody framework that includes constant regions of any species, including human constant regions, where expression of the construct in a cell can produce a single antibody molecule or antigen-binding protein that is referred to herein as monoclonal.

The modifier “monoclonal” thus indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In various embodiments, an antibody specifically binds to a target antigen if it binds to the antigen with a dissociation constant (K_{d}) of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ or less, or 10⁻¹² or less.

Binding affinity of an antigen-binding protein for a target antigen can be reported as a dissociation constant (K_{d}) which can be measured using a surface plasmon resonance (SPR) assay. Surface plasmon resonance refers to an optical phenomenon that allows for the analysis of real-time interactions by detection of alterations in protein concentrations within a biosensor matrix, for example using a BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

With respect to antibodies, the term “antagonist” and “antagonistic” refers to a blocking antibody that binds its cognate target antigen and inhibits or reduces the biological activity of the bound antigen. The term “agonist” or “agonistic” refers to an antibody that binds its cognate target antigen in a manner that mimics the binding of the physiological ligand which causes antibody-mediated downstream signaling.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)2 fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain. In one embodiment, the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-Spike protein antibodies described herein.

A “humanized antibody” refers to an antibody originating from a non-human species that has one or more variable and constant regions that has been sequence modified to conform to corresponding human immunoglobulin amino acid sequences. For example, the constant regions of a humanized antibody may be human constant region sequences, where the amino acid sequence of a variable domains may be from an antibody sequence of another species, such as a mouse (in which the antibody may have been generated). A humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In some embodiments, the constant domain(s) from a human antibody are fused to the variable domain(s) of a non-human species. In some embodiments, one or more amino acid residues in one or more CDR sequences of a non-human antibody is changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

In some embodiments, an antibody can be a “fully human” antibody in which all of the constant and variable domains (optionally excepting from the CDRs) are derived from human immunoglobulin sequences. A fully human antibody as disclosed herein may have one or more mutations (which may be, for example amino acid substitutions, deletions, or insertions) in the constant regions, such as for example the Fc constant regions of the heavy chain, with respect to a wild type human antibody sequence. For example, a fully human antibody can have one or more mutation in the constant regions of either the light or heavy chain of the antibody, where the sequence of either or both of the light chain constant region or heavy chain constant regions (CH1, CH2, and CH3) of the fully human antibody are greater than 95%, greater than 96%, greater than 97%, and preferably greater than 98% or at least 99% identical to the sequence of the non-mutant human constant regions. Humanized and fully human antibodies may be prepared in a variety of ways, examples of which are described below, including through recombinant methodologies or through immunization with an antigen of interest of a mouse that is genetically modified to express antibodies derived from human heavy and/or light chain-encoding genes, e.g., the “Xenomouse II” that, when challenged with an antigen, generates high affinity fully human antibodies Mendez et al. ((1997) Nature Genetics 15: 146-156). This was achieved by germ-line integration of megabase human heavy chain and light chain loci into mice with deletion of the endogenous JH region. The antibodies produced in these mice closely resemble that seen in humans in all respects, including gene rearrangement, assembly, and repertoire.

Alternatively, phage display technology (McCafferty et al., Nature 348, 552-553 [1990]) can be used to produce human antibodies and antibody fragments in vitro, from immunoglobulin variable (V) domain gene repertoires from immunized or nonimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; see, e.g., Johnson, Kevin S. and Chiswell, David J., Current Opinion in Structural Biology 3, 564-571 (1993). Any of a number of sources of V-gene segments can be used for phage display, e.g., the spleens of immunized mice (Clackson et al., Nature 352, 624-628 (1991)) or blood cells of nonimmunized human donors can be used to generate antibodies to a diverse array of antigens (including self-antigens) can be isolated essentially following the techniques described by Marks et al., J. Mol. Biol. 222, 581-597 (1991) or Griffith et al., EMBO J. 12, 725-734 (1993).

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions of a chimeric antibody may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides. As used herein a “variant” of a virus, such as SARS-CoV-2, is a virus that has differentiated in sequence from the originally identified strain (e.g., Wuhan-Hu-1 SARS-CoV-2), typically by variation arising in a population through naturally-occurring mutation(s) of the original strain. A variant virus can differ from the original, or parental, strain by about 1% to 10% or more of its nucleotide sequence and may differ from the parental strain by one or more amino acids in one or more open reading frames encoded by the viral genome. For example, one or more polypeptides encoded by a variant virus can have 99% or less, 98% or less, 97% or less, 96% or less, 95% or less, 94% or less, 93% or less, 92% or less, 91% or less, or 90% or less amino acid sequence identity to the parental or reference virus.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation.

Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8a hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 45). In one embodiment, the hinge region comprises an IgG1 core hinge sequence CPXC, wherein X is P, R or S. In one embodiment, the hinge region comprises a lower hinge/CH2 sequence PAPELLGGP (SEQ ID NO:18). In one embodiment, the hinge is joined to an Fc region (CH2) having the amino acid sequence SVFLFPPKPKDT (SEQ ID NO:19). In one embodiment, the hinge region includes the amino acid sequence of an upper, core and lower hinge and comprises EPKSCDKTHTCPPCPAP ELLGGP (SEQ ID NO:20). In one embodiment, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions and may, or may not, include a portion of the hinge region. An Fc domain can bind Fc cell surface receptors and some proteins of the immune complement system. An Fc region can bind a complement component C1q. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a). In one embodiment, the Fc region can include a mutation that increases or decreases any one or any combination of these functions. In one embodiment, the Fc domain comprises a LALA mutation (e.g., equivalent to L234A, L235A according to Kabat numbering) which reduces effector function (see, for example Hezareh et al. (2001) J Virol 12161-12168). In some embodiments, the Fc domain comprises a LALA-PG mutation (e.g., equivalent to L234A, L235A, P329G according to Kabat numbering) which reduces effector function (e.g., Lo et al. (2017) J Biol Chem. 292:3900-3908). In some embodiments, a mutation in the Fc domain (such as but not limited to the YTE mutations) can increase or decrease the serum half-life of the protein complex and/or can increase or decrease the thermal stability of the protein complex.

The term “labeled” or related terms as used herein with respect to a polypeptide refers to joinder antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-PD-1 antibodies described herein can be unlabeled or can be joined to a detectable label or moiety.

The term “labeled” or related terms as used herein with respect to a polypeptide refers to joinder thereof to a detectable label or moiety for detection. Exemplary detectable labels or moieties include radioactive, colorimetric, antigenic, enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-spike protein antibodies described herein or antigen-binding portions thereof that described herein can be unlabeled or can be joined to a detectable label or detectable moiety.

A “neutralizing antibody” and related terms refers to an antibody that is capable of specifically binding to a target antigen (e.g., a coronavirus spike protein) and substantially inhibiting or eliminating the biological activity of the target antigen. In the present context, a neutralizing antibody binds to a coronavirus and inhibits infection of susceptible cells by the coronavirus. An antibody that blocks binding of the coronavirus to a target cell is a neutralizing antibody as binding is required for infection of the target cell. As provided herein, a “neutralizing antibody”, an “antibody with neutralizing activity”, or “inhibitory antibody” is an antibody that neutralizes 100 times the tissue culture infectious dose required to infect 50% of cells (100×TCID₅₀) of a virus, for example, a SARS coronavirus, such as for example, SARS-CoV-1 or SARS-CoV-2. In some embodiments, a “neutralizing antibody” is an antibody that neutralizes 200 times the tissue culture infectious dose required to infect 50% of cells (200×TCID₅₀) of a virus, for example, a SARS Corona virus, such as SARS-CoV-1 or SARS-CoV-2. Neutralizing antibodies such as those disclosed herein are effective at antibody concentrations of less than 20 μg/ml, less than 15 μg/ml, less than 12.5 μg/ml, less than 10 μg/ml, less than 5 μg/ml, less than 3.5 μg/ml, less than 2 μg/ml or less than 1 μg/ml. In some preferred embodiments, neutralizing antibodies are effective at antibody concentrations of <0.8 μg/ml. In some preferred embodiments, neutralizing antibodies are effective at antibody concentrations of less than 0.5 μg/ml and in some further preferred embodiments, neutralizing antibodies are effective at antibody concentrations of less than 0.2 μg/ml or less than 0.1 μg/ml.

The term “TCID₅₀” or “median tissue culture infective dose” refers to the amount of virus necessary to infect 50% of cells in tissue culture. The 100× and 200× refer to 100 and 200 times the TCID₅₀ concentration of virus.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not necessarily identical to any of the amino acid sequences of the polypeptides that make up any of the anti-spike protein antibodies, or antigen binding protein thereof, described herein. The similarities between the test antibody and the polypeptides can be at least 95%, or at or at least 96% identical, or at least 97% identical, or at least 98% identical, or at least 99% identical, to any of the polypeptides that make up any of the anti-spike protein antibodies, or antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Expression vectors can include one or more origin of replication sequences. Vectors can be single-stranded or double-stranded nucleic acid molecules. Vectors can be linear or circular nucleic acid molecules. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated viral, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequences. Regulatory sequences direct transcription, or transcription and translation, of a transgene linked to or inserted into the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606.

A transgene is “operably linked” to a regulatory sequence (e.g., a promoter) when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell, such as an antibody production host cell. A “transfected” or “transformed” or “transduced” host cell is one which has been introduced with exogenous nucleic acid (transgene). The host cell includes the primary subject cell and its progeny. Exogenous nucleic acids encoding at least a portion of any of the anti-spike protein antibodies described herein can be introduced into a host cell. Expression vectors comprising at least a portion of any of the anti-spike protein antibodies described herein can be introduced into a host cell, and the host cell can express polypeptides comprising at least a portion of the anti-spike protein antibody.

In this context, a host cell can be a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with a nucleic acid either to be expressed or not to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Thus the terms “host cell” or “or a population of host cells” or related terms as used herein may refer to a cell (or a population thereof or a plurality of host cells) to be used for production of the antibody or fragment thereof, is a cell or cells into which foreign (exogenous or transgene) nucleic acids have been introduced, for example, to direct production of the anti-spike protein antibody by the production host cell. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell, can be extracted from a subject, or can be the cell of an organism, including a human subject. The host cell (or a population of host cells) includes the primary subject cell and its progeny without any regard for the number of generations or passages. The host cell (or a population thereof) includes immortalized cell lines. Progeny cells may or may not harbor identical genetic material compared to the parent cell. In one embodiment, a production host cell describes any cell (including its progeny) that has been modified, transfected, transduced, transformed, and/or manipulated in any way to express an antibody, as disclosed herein. In one example, the host cell (or population thereof) can be transfected or transduced with an expression vector operably linked to a nucleic acid encoding the desired antibody, or an antigen binding portion thereof, as described herein. Production host cells and populations thereof can harbor an expression vector that is stably integrated into the host's genome or can harbor an extrachromosomal expression vector. In one embodiment, host cells and populations thereof can harbor an extrachromosomal vector that is present after several cell divisions or is present transiently and is lost after several cell divisions.

In other contexts, the disclosure may use the term “host cell” or “host cells” to refer to a cell or cells that are infected with a virus (such as a coronavirus), cells capable of being infected by a virus (e.g., lung cells of a subject), or cells used in assays or experiments testing their ability to be infected by a virus. Other terms for virally-infected cells, cells capable of being infected by a virus, or cells used in assays that include viral infection procedures, may include, as nonlimiting examples, “target cells”, “susceptible cells”, “test cells”, “virus propagating cells”, “infected cells”, and the like.

The term “subject” as used herein refers to human and non-human animals, including vertebrates, mammals and non-mammals. In one embodiment, the subject can be human, non-human primates, simian, ape, murine (e.g., mice), bovine, porcine, equine, canine, feline, caprine, lupine, ranine, or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually, or topically. Intranasal delivery can be by topical administration of a liquid, paste, gel, or powder formulation applied to the nasal passages. Deposition of a liquid, paste, gel, or powder formulation within the nasal passages that may subsequently lead to inhalation of the antibody therapeutic provided in the composition which may then be delivered to the lung. Administering can also be performed, for example, once, a plurality of times, and/or over one or more extended periods. Any of the anti-spike protein neutralizing antibodies described herein (or antigen binding protein thereof) can be administered to a subject using art-known methods and delivery routes.

The terms “effective amount”, “therapeutically effective amount” or “effective dose” or related terms may be used interchangeably and refer to an amount of antibody or an antigen binding protein (e.g., any of the anti-spike protein antibodies described herein or antigen binding protein thereof) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered to a subject at about 0.01 g/kg-50 mg/kg per day, about 0.01 mg/kg-30 mg/kg per day, or about 0.1 mg/kg-20 mg/kg per day. The polypeptide may be administered daily (e.g., once, twice, three times, or four times daily) or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject testing positive for a coronavirus infection, such as an infection with SARS-CoV or SARS-CoV-2. The present disclosure also provides methods for treating a subject suspected of being infected or at risk of being infected with a coronavirus, such as SARS-CoV or SARS-CoV-2.

Antigen Binding Proteins that Specifically Bind the Coronavirus S1 Protein

The present disclosure provides antigen-binding proteins that specifically bind the spike (S) protein of a beta coronavirus, such as but not limited to the S protein of HCoV-NL63, SARS-CoV, or SARS-CoV-2. The term spike protein or S protein, as used herein, includes both the precursor form of an S protein that includes the N-terminal leader sequence and the processed or mature form that lacks the N-terminal leader sequence. Thus an antigen-binding protein that binds an S protein can bind both the precursor that includes the leader sequence and the mature S protein that lacks the N-terminal leader sequence, but preferably binds at least the mature processed form of the S protein of a coronavirus such as the S protein of SARS-CoV-2. (The leader sequence of the SARS-CoV-2 S protein is not precisely defined, but may extend from the N-terminus to amino acid 11, 12, 13, 14, 15, or 16 of SEQ ID NO:1, for example from the N-terminus to amino acid 12, 13, 14, or 15 of SEQ ID NO:1.) An antigen-binding protein as provided herein can specifically bind the spike protein of SARS-CoV-2 having the amino acid sequence of SEQ ID NO:1 or comprising the amino acid sequence of SEQ ID NO:2, and in various embodiments can specifically bind an S protein of a coronavirus where the S protein comprises an amino acid sequence having at least 95%, at least 96%, at least 97%, at least 98%, or at least 99% identity to the amino acid sequence of SEQ ID NO:1 or SEQ ID NO:2.

The SARS-CoV2 coronavirus spike protein, a transmembrane protein having an ectodomain that extends from the N-terminus of the mature protein to amino acid 1208 of SEQ ID NO:1, has two regions or domains, referred to as S1 and S2, that are cleaved into the S1 and S2 subunits after binding of the S1 domain, which includes the receptor binding domain (RBD) of the S protein, to the ACE2 protein on target cells. In general, the term “S1 subunit” or “S1 protein” as used herein will refer to the cleaved S1 region of the spike or “S” protein of a coronavirus, i.e., a separate polypeptide.

In various embodiments the antigen-binding protein disclosed herein specifically binds the S1 subunit of the SARS-CoV-2 coronavirus. For example, an antigen-binding protein as provided herein can specifically bind the S1 subunit or the S1 domain of a spike protein of SARS-CoV-2 having the amino acid sequence of SEQ ID NO:4 and in various embodiments can specifically bind an S1 subunit of a coronavirus having at least 95%, at least 96%, at least 97%, at least 98%, or at least 99% sequence identity to the amino acid sequence of SEQ ID NO:4.

In various embodiments the antigen-binding protein disclosed herein specifically binds the RBD of the S1 subunit of the SARS-CoV-2 coronavirus. For example, an antigen-binding protein as provided herein can specifically bind the RBD of a spike protein of SARS-CoV-2 having the amino acid sequence of SEQ ID NO:5 and in various embodiments can specifically bind an S1 subunit of a coronavirus having at least 95%, at least 96%, at least 97%, at least 98%, or at least 99% sequence identity to the amino acid sequence of SEQ ID NO:5.

The antigen-binding proteins provided herein that bind the S protein of a coronavirus such as SARS-CoV-2 can comprise an amino acid sequence having at least 95%, at least 96%, at least 97%, at least 98%, or at least 99% sequence identity to the amino acid sequence of SEQ ID NO:28 (the heavy chain variable region of antibody S1D7270) and an amino acid sequence having at least 95%, at least 96%, at least 97%, at least 98%, or at least 99% sequence identity to the amino acid sequence of SEQ ID NO:29 (the light chain variable region of antibody S1D7270). Alternatively or in addition, the antigen-binding proteins provided herein that specifically bind the S protein of a betacoronavirus, such as but not limited to the S protein of SARS-CoV-2, include the heavy chain complementarity-determining regions (CDRs) of SEQ ID NO:30 (heavy chain CDR1), SEQ ID NO:31 (heavy chain CDR2), and SEQ ID NO:32 (heavy chain CDR3) and further include the light chain complementarity-determining regions (CDRs) of SEQ ID NO:33 (light chain CDR1), SEQ ID NO:34 (light chain CDR2), and SEQ ID NO:35 (light chain CDR3). Antigen-binding proteins as provided herein that include the CDR sequences of SEQ ID NO:30, SEQ ID NO:31, SEQ ID NO:32, SEQ ID NO:33, SEQ ID NO:34, and SEQ ID NO:35 can in some embodiments include a heavy chain variable region sequence having at least 95%, at least 96%, at least 97%, at least 98%, or at least 99% identity to the amino acid sequence of SEQ ID NO:28 and a light chain variable region sequence having at least 95%, at least 96%, at least 97%, at least 98%, or at least 99% identity to the amino acid sequence of SEQ ID NO:29. In some exemplary embodiments of the antigen-binding proteins provided herein that specifically bind the S protein of a betacoronavirus, such as the S protein of SARS-CoV-2, the antigen-binding proteins include the heavy chain variable region amino acid sequence of SEQ ID NO:28 and the light chain variable region amino acid sequence of SEQ ID NO:29. In various embodiments the antigen-binding protein is an antibody or an antibody fragment.

The antigen-binding proteins provided herein having an amino acid sequence having at least 95% sequence identity to the amino acid sequence of SEQ ID NO:28 and an amino acid sequence having at least 95% sequence identity to the amino acid sequence of SEQ ID NO:29 and/or comprising the heavy chain CDR1 sequence of SEQ ID NO:30, the heavy chain CDR2 sequence of SEQ ID NO:31, the heavy chain CDR3 sequence of SEQ ID NO:32, the light chain CDR1 sequence of SEQ ID NO:33, the light chain CDR2 sequence of SEQ ID NO:34, and the light chain CDR3 sequence of SEQ ID NO:35 can bind the S protein of a coronavirus such as the S protein of SARS-CoV-2 with a binding affinity (K_{d}) of 10⁻⁵ M (10 μM) or less, 10⁻⁶ M (1 μM) or less, 10⁻⁷ M (100 nM) or less, 5×10⁻⁸ M (50 nM) or less, 10⁻⁸ M (10 nM) or less, 10⁻⁹ M (1 nM) or less, or 10⁻¹⁰ M (0.1 nM) or less. For example, the antigen-binding protein can bind the S protein of a coronavirus such as SARS-Cov-2 with a K_{d} of between 10⁻⁵ M and 10⁻⁶ M, or between 10⁻⁶ M and 10⁻⁷ M, or between 10⁻⁷ M and 10⁻⁸ M, or between 10⁻⁸ M and 10⁻⁹ M, between 10⁻⁹ M and 10⁻¹⁰ M, or between 10⁻¹⁰ M and 10⁻¹¹ M.

For example, an antigen-binding protein as provided herein that specifically binds the S protein of SARS-CoV-2, which can be, as nonlimiting examples, an IgG, a Fab fragment, or a single chain antibody, or can be an antigen-binding protein comprising or derived from any thereof, can specifically bind a coronavirus spike protein (e.g., a spike protein comprising the amino acid sequences of SEQ ID NO:1, comprising amino acids 16-1273 of SEQ ID NO:1, or comprising SEQ ID NO:2, or a spike protein of a coronavirus comprising an amino acid sequence having at least 95%, 96%, 97%, 98%, or 99% identity to SEQ ID NO:1, amino acids 16-1273 of SEQ ID NO: 1, or SEQ ID NO:2) with a K_{d} of between about 200 nM and about 0.01 nM, or between 100 nM and 0.1 nM, or between 10 nM and 0.1 nM. In some embodiments, the antigen-binding protein is the S1D7270 antibody having a heavy chain variable sequence of SEQ ID NO:28 and a light chain variable sequence of SEQ ID NO:29, and optionally comprising one or more mutations in the Fc region, having a K_{d} for binding the S protein of SARS-CoV-2 of between about 10 nM and about 0.1 nM or between about 5 nM and about 0.5 nM.

In some embodiments the antigen-binding protein can bind the S1 subunit of the S protein of a coronavirus such as the S1 subunit of SARS-CoV-2 with a binding affinity (K_{d}) of 10⁻⁵ M or less, or 10⁻⁶ M or less, or 10⁻⁷ M or less, or 10⁻⁸ M or less, or 10⁻⁹ M or less, or 10⁻¹⁰ M or less. For example, the antigen-binding protein can bind the S1 subunit of an S protein of a coronavirus such as SARS-CoV-2 with a K_{d} of between 10⁻⁵ M and 10⁻⁶ M, or between 10⁻⁶ M and 10⁻⁷ M, or between 10⁻⁷ M and 10⁻⁸ M, or between 10⁻⁸ M and 10⁻⁹ M, or between 10⁻⁹ M and 10⁻¹⁰ M. For example, an antigen-binding protein provided herein, which can in various embodiments be an antibody, such as a fully human antibody, and may be, as nonlimiting examples, an IgG, a Fab fragment, or a single chain antibody, or can be an antigen-binding protein derived from any thereof, specifically binds the S1 subunit of a coronavirus S protein (e.g., SEQ ID NO:4 or an S1 subunit of a spike protein of a coronavirus comprising a sequence having at least 95%, 96%, 97%, 98%, or 99% identity to SEQ ID NO:4) with a K_{d} of between about 200 nM and about 0.01 nM, or between 100 nM and 0.1 nM, or between 10 nM and 0.1 nM. In some embodiments, the antibody is the fully human S1D7270 antibody or is an antibody fragment or antigen-binding protein derived therefrom, having a variable heavy chain sequence of SEQ ID NO:28 and a variable light chain sequence of SEQ ID NO:29, that binds the S1 subunit of the S protein of SARS-CoV-2 with a K_{d} of between about 10 nM and about 0.1 nM or between about 5 nM and about 0.5 nM. In some embodiments the antibody is a fully humanized IgG having a variable heavy chain sequence of SEQ ID NO:28 and a variable light chain sequence of SEQ ID NO:29 that optionally comprises one or more mutations in the Fc region. In some embodiments the one or more mutations in the Fc region is a mutation that reduces ADE, such as the LALA (Leu234Ala, Leu235Ala) mutations.

In some embodiments the antigen-binding protein can bind the RBD of the S protein of a coronavirus (which is localized to the S1 domain) such as the RBD of the SARS-CoV S protein or SARS-CoV-2 S protein with a binding affinity (K_{d}) of 10⁻⁵ M or less, or 10⁻⁶ M or less, or 10⁻⁷ M or less, or 10⁻⁸ M or less, or 10⁻⁹ M or less, or 10⁻¹⁰ M or less. For example, the antigen-binding protein in various embodiments can bind the RBD of an S protein of a coronavirus such as SARS-CoV-2 with a K_{d} of between 10⁻⁵ M and 10⁻⁶ M, or between 10⁻⁶ M and 10⁻⁷ M, or between 10⁻⁷ M and 10⁻⁸ M, or between 10⁻⁸ M and 10⁻⁹ M, or between 10⁻⁹ M and 10⁻¹⁰ M. In some embodiments, an antigen-binding protein provided herein, which can be in various embodiments be an antibody, such as a fully human antibody, and may be, as nonlimiting examples, an IgG, a Fab fragment, or a single chain antibody, or can be an antigen-binding protein derived from any thereof, specifically binds the receptor binding domain (RBD) of a coronavirus S protein (e.g., SEQ ID NO:5 or an RBD of a spike protein of a coronavirus comprising an amino acid sequence having at least 95%, 96%, 97%, 98%, or 99% identity to SEQ ID NO:5) with a K_{d} of between of between about 200 nM and about 0.01 nM, or between 100 nM and 0.1 nM, or between 10 nM and 0.1 nM. In some embodiments, the antigen binding protein is the fully human S1D7270 antibody having a variable heavy chain sequence of SEQ ID NO:28 and a variable light chain sequence of SEQ ID NO:29, and optionally comprising one or more mutations in the Fc region, that binds the RBD of the S protein of SARS-CoV-2 with a K_{d} of between about 10 nM and about 0.1 nM or between about 5 nM and about 0.1 nM. For example, the antigen binding protein may be the fully human S1D7270 antibody disclosed herein having a variable heavy chain sequence of SEQ ID NO:28 and a variable light chain sequence of SEQ ID NO:29, and optionally having L234A and L235A mutations in the Fc region, where the antibody binds the RBD of the S protein of SARS-CoV-2 with a K_{d} of between about 10 nM and about 0.1 nM, or between about 5 nM and about 0.5 nM, or between about 2 nM and about 1 nM, as measured by SPR.

Binding affinity of an antigen-binding protein as provided herein to an S protein, S1 subunit, or RBD can be calculated based on binding assays performed using methods known in the art such as surface plasmon resonance (SPR). The sequences of many coronavirus proteins are publicly available such that the proteins can be produced using recombinant methods and many, including for example coronavirus S proteins, S1 subunits, and the RBDs of S proteins, are commercially available. Commercial sources for coronavirus proteins include for example Sino Biological US (Wayne, PA) and Acrobiosystems (San Jose, CA, USA).

In various embodiments, the antigen-binding proteins described herein block binding between the S protein of a coronavirus (such as HCoV-NL63, SARS-CoV, or SARS-CoV-2) and the ACE2 protein, for example, block binding of the ectodomain of the human ACE2 protein (hACE2) by the S protein of a coronavirus. In various embodiments, the antigen-binding proteins described herein can block binding between the S protein of SARS-CoV-2 and the human ACE2 protein with an IC₅₀ of between about 0.001 μg/ml and about 200 μg/ml, between about 0.01 μg/ml and about 100 μg/ml, between about 0.05 μg/ml and about 50 μg/ml, between about 0.1 μg/ml and about 10 μg/ml, between about 1 μg/ml and about 10 μg/ml, or between about 1 μg/ml and about 50 μg/ml, between about 0.1 μg/ml and about 5 μg/ml, or between about 0.1 μg/ml and about 1 μg/ml. In some embodiments, the antigen binding proteins described herein block binding between the S1 domain or subunit of SARS-CoV-2 and the human ACE2 protein with an IC₅₀ of between about 0.001 μg/ml and about 200 μg/ml, between about 0.01 μg/ml and about 100 μg/ml, between about 0.05 μg/ml and about 50 μg/ml, between about 0.1 μg/ml and about 10 μg/ml, between about 1 μg/ml and about 10 μg/ml, or between about 1 μg/ml and about 50 μg/ml, between about 0.1 μg/ml and about 5 μg/ml, or between about 0.1 μg/ml and about 1 μg/ml. For example, an antigen-binding protein as disclosed herein can block the binding of the S1 subunit of SARS-CoV-2 and the human ACE2 protein with an IC₅₀ of less than 200 nM, less than 100 nM, less than 50 nM, less than 10 nM, less than 5 nM, or less than 1 nM, for example, with an IC₅₀ of between about 100 nM and about 0.1 nM or between about 50 nM and about 0.5 nM, or between about 20 nM and about 0.2 nM or between about 50 nM and about 1 nM, for example, between about 10 nM and about 1 nM, or between about 5 nM and about 0.5 nM.

In various embodiments provided herein, an antigen-binding protein that specifically binds the S protein, S1 subunit, or S protein RBD of a coronavirus as disclosed herein is a neutralizing antibody that it is able to inhibit infection of a target cell by a coronavirus such as the SARS-CoV virus or the SARS-CoV-2 virus. For example, as demonstrated herein, an antigen-binding protein as provided herein, such as but not limited to the S1D7270 fully human antibody, optionally including a LALA mutation in the Fc region (e.g., antibody STI-2020), can inhibit the cytopathic effect (CPE) on susceptible cells at a concentration of between about 0.1 ng/mL and about 500 ng/mL, or between about 1 ng/mL and 200 ng/mL, or between about 5 ng/mL and about 100 ng/mL or about 10 ng/mL and about 80 ng/mL, or between about 45 ng/mL and about 65 ng/mL.

An antigen-binding protein as provided herein can be or can be derived from an antibody, such as an IgG, IgA, IgD, IgE, or IgM antibody that specifically binds the S protein of SARS-CoV-2. In some embodiments, an IgG, IgA, IgD, IgE, or IgM antibody as provided herein, or an antigen-binding protein derived therefrom, can specifically bind the S protein of a coronavirus (such as but not limited to SARS-CoV or SARS-CoV-2 or a variant of SARS-CoV or SARS-CoV-2) having an S protein with at least 65%, at least 70%, at least 75%, at least 80%, at least 85%, at least 90%, at least 95%, at least 96%, at least 97%, at least 98%, or at least 99% amino acid sequence identity to SEQ ID NO:1 or SEQ ID NO:2. In various embodiments an IgG, IgA, IgD, IgE, or IgM antibody as provided herein, or an antigen-binding protein derived therefrom, can specifically bind the S1 subunit of a coronavirus (such as but not limited to SARS or SARS-CoV-2 or a variant of SARS or SARS-CoV-2) having an S1 subunit with at least 65%, at least 70%, at least 75%, at least 80%, at least 85%, at least 90%, at least 95%, at least 96%, at least 97%, at least 98%, or at least 99% amino acid sequence identity to SEQ ID NO:4. In some embodiments the antigen-binding protein specifically binds the RBD that is within the S1 domain of the S protein.

An antigen-binding protein as provided herein that specifically binds the S protein of a coronavirus can be or comprise an antibody, such as for example an IgG, IgA, IgD, IgE, IgM, or a single chain antibody and/or can be or can comprise antibody fragments, such as but not limited to Fab, Fab′, or F(ab′)2 antibody fragments. In some embodiments, an antigen-binding protein as provided herein comprises an IgG or IgM antibody. In some embodiments, an antigen-binding protein comprises an IgG1, IgG2, IgG3, or IgG4 antibody. In some embodiments, an antigen-binding protein comprises an IgG1 or IgG4 antibody. For example, an antigen-binding protein as provided herein can be an IgG1 or IgG4 antibody having an ADE-reducing mutation, such as the LALA mutation, in the Fc region, or can be single chain antibody (e.g., an scFv or scFab) that optionally includes an Fc region that can optionally include an ADE-reducing mutation, such as the LALA mutation. In further embodiments, an antigen-binding protein as provided herein can be a Fab, Fab′, or F(ab′)2 antibody fragment.

An antigen-binding protein as provided herein that is “derived from” an IgG, IgA, IgD, IgE, or IgM antibody as provided herein that specifically binds the S protein of a coronavirus can have heavy chain CDR amino acid sequences of SEQ ID NO:30, SEQ ID NO:31, and SEQ ID NO:32 and light chain CDR amino acid sequences of SEQ ID NO:33, SEQ ID NO:34, and SEQ ID NO:35 or can have a heavy chain sequence having at least 95% identity to SEQ ID NO:28 and a light chain sequence having at least 95% identity to SEQ ID NO:29. For example, an antigen-binding protein derived from an antibody as provided herein that specifically binds the S protein of a coronavirus can include the heavy chain CDR amino acid sequences of SEQ ID NO:30, SEQ ID NO:31, and SEQ ID NO:32 and light chain CDR amino acid sequences of SEQ ID NO:33, SEQ ID NO:34, and SEQ ID NO:35 and can further include an amino acid sequence having at least 95% identity to SEQ ID NO:28 (the heavy chain variable domain of antibody S1D7270) and an amino acid sequence having at least 95% identity to SEQ ID NO:29 (the light chain variable domain of antibody S1D7270). The antigen-binding protein can have neutralizing activity against a coronavirus such as SARS-CoV-2 or a variant thereof. In various embodiments the S protein-binding protein as provided herein comprises or is derived from an IgG1, IgG2, IgG3, or IgG4 antibody. For example, the antigen-binding protein that binds an S protein can comprise or be derived from an IgG1 or IgG4 class antibody. In further embodiments, an antigen-binding protein as provided herein can be or comprise an antibody fragment, such as but not limited to a Fab fragment, a Fab′ fragment, or F(ab′)2 fragment. In additional embodiments an antigen-binding protein as provided herein can be or comprise a single chain antibody (e.g., an ScFv). An antigen binding protein as provided herein that may be or may not be an immunoglobulin molecule can comprise more than one polypeptide chain where the two or more polypeptide chains assemble together, for example, can be an immunoglobulin having two heavy and two light chains, or can be a multi-subunit protein having one heavy and one light chain, or can be a non-immunoglobulin protein that comprises multiple polypeptide chains that may comprise, for example, one or more Fab fragments or ScFvs, etc.

Antigen-binding proteins as provided herein can comprise or be derived from antibodies, antibody fragments, and single chain antibodies. For example, an antigen-binding protein as provided herein can include heavy chain variable region and light chain variable region sequences of an antibody disclosed herein, such as the S1D7270 antibody, or sequences having at least 95% amino acid sequence identity thereto, or can include CDR sequences of an antibody disclosed herein, where the antigen-binding protein can have a framework that is not an antibody framework, or the antigen binding protein can include additional protein moieties that may be, as nonlimiting examples, active domains or additional binding domains.

In some nonlimiting embodiments, the antigen-binding protein provided herein is or comprises a fully human antibody or a fully human antibody fragment, for example, a fully human IgG1, IgG2, IgG3, IgG4, IgA, IgD, IgE, or IgM, or a fully human single chain antibody, fully human Fab fragment, or fully human single chain antibody, or is or comprises an antigen binding protein derived from or comprising any of these.

In some embodiments the antigen-binding protein is an IgG, IgA, IgD, IgE, or IgM antibody having one or more mutations in the Fc region, for example one or more mutations that decreases antibody dependent enhancement (ADE) and/or one or more mutations that increases antibody half-life. ADE can occur when the presence of virus-specific antibodies increase pathogenicity of a virus by enhancing the entry of virus due to binding of IgG Fc regions to Fc-receptors on the surface of cells that naturally bind Fc regions of antibodies such as macrophages, monocytes, and dendritic cells (see, for example, Khandia et al. (2018) Frontiers Immunol. 9; doi:103389/fimmu.2018.00597). Mutations that reduce or eliminate interaction of the Fc region of antibody with its receptor (e.g., FcγRs) on such cells can reduce or eliminate ADE.

In some embodiments the antibody has one or more mutations in the Fc region selected from L234A; L235A or L235E; N297A, N297Q, or N297D; and P329A or P329G. For example, the anti-S antibody can include the mutations L234A and L235A (LALA). An antibody having one or more mutations in the Fc region selected from L234A; L235A or L235E; N297A, N297Q, or N297D; and P329A or P329G can demonstrate reduced ADE with respect to the same antibody without the mutation(s). For example, an antibody as provided herein that includes the LALA mutations can have reduced ADE with respect to the antibody without the LALA mutations.

Alternatively or in addition, the antibody may have one or more mutations in the Fc region that increase half-life of the antibody in human serum. Antibody therapeutics administered by the intravenous route may have a typical serum half-life of approximately 20 days. Mutations such as the M252Y/S254T/T256E (YTE) mutation in the Fc region of an antibody has a serum half-life of up 100 days (e.g., US 2011/0158985, incorporated by reference in its entirety). Extending the half-life a neutralizing antibody as provided herein that binds the S protein of a coronavirus such as SARS-CoV-2 can improve its usefulness as a prophylactic to confer prolonged passive immunity to those that are regularly exposed to the virus (e.g. healthcare workers) or individuals at particular risk following an exposure event (e.g. the elderly, patients with hypertension, or immune-compromised patients). In various examples, an neutralizing antibody the specifically binds the S protein of a coronavirus such as SARS-CoV-2, can have an Fc region mutation that extends the half-life of the antibody such as any selected from M252Y, S254T, T256D or T256E, T307Q or T307W, M428L, and N434S. For example, the anti-S antibody can include the mutations M252Y, S254T, and T256E (YTE).

An antigen-binding protein as provided herein that specifically binds a coronavirus S protein, including an antibody or antibody fragment that specifically binds a coronavirus S protein, can include a label, such as, for example, a fluorophore. An antigen-binding protein as disclosed herein can further include one or more additional amino acid sequences in addition to antibody-derived sequences (e.g., heavy chain and/or light chain sequences), such as but not limited to peptide tags, localization sequences, linkers, fluorescent protein sequences, or functional domains, including additional binding domains or enzymatic domains. Peptide or protein tags that can be used for detection and/or purification of the antigen-binding protein can include, without limitation, his, FLAG, a myc, HA, V5, polyarginine, calmodulin, a maltose binding protein tag, a GST tag, and a Halo tag.

In some examples an antigen-binding protein as provided herein that specifically binds the S protein of the SARS-CoV-2 coronavirus has a heavy chain variable domain having the sequence of SEQ ID NO:28 and a light chain variable domain having the sequence of SEQ ID NO:29. In some embodiments an antigen binding protein as provided herein is or comprises an IgG antibody having the heavy chain variable region of SEQ ID NO:28 and the light chain variable region of SEQ ID NO:29. The antibody can specifically bind the S1 subunit of the coronavirus S protein, and in some embodiments specifically binds the RBD of the S protein. In some embodiments, the present disclosure provides a fully human antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to SEQ ID NO:28 and SEQ ID NO:29. In some embodiments an antigen binding protein as provided herein is or comprises a monoclonal fully human IgG antibody having the heavy chain variable region of SEQ ID NO:28 and the light chain variable region of SEQ ID NO:29 (herein called S1D7270).

As disclosed herein, antibody S1D7270 is a fully human recombinant monoclonal IgG1 antibody that is a variant of parent antibody S1D2 (which has a heavy chain variable domain having the sequence of SEQ ID NO:6 and a light chain variable domain having the sequence of SEQ ID NO:7), where variant antibody S1D7270 has a heavy chain variable domain having the sequence of SEQ ID NO:28 and a light chain variable domain having the sequence of SEQ ID NO:29. The S1D7270 antibody has a heavy chain CDR1 with the amino acid sequence of SEQ ID NO:30, a heavy chain CDR2 with the amino acid sequence of SEQ ID NO:31, a heavy chain CDR3 with the amino acid sequence of SEQ ID NO:32, a light chain CDR1 with the amino acid sequence of SEQ ID NO:33, a light chain CDR2 with the amino acid sequence of SEQ ID NO:34, and a light chain CDR3 with the amino acid sequence of SEQ ID NO:35. As disclosed in the examples herein, the S1D7270 antibody specifically binds the RBD of the S1 subunit protein of the SARS-CoV-2 coronavirus (SEQ ID NO:5) with a K_{d} of between about 10 nM and about 0.1 nM, for example, of between about 5 nM and about 0.5 nM, or about 1.3 nM. In some embodiments the S1D7270 antibody includes the mutations L234A and L235A in the Fc region (S1D7270_{LALA}, referred to in the Examples herein as STI-2020).

The present disclosure also provides a Fab fully human antibody fragment, comprising a heavy variable region from a heavy chain and a variable region from a light chain, wherein the sequence of the variable region from the heavy chain is at least 95% identical, or at least 96% identical, or at least 97% identical, or at least 98% identical, or at least 99% identical to the amino acid sequence of SEQ ID NO:28. The sequence of the variable region from the light chain can be at least 95% identical, or at least 96% identical, or at least 97% identical, or at least 98% identical, or at least 99% identical to the amino acid sequence of SEQ ID NO:29.

The present disclosure further provides a single chain fully human antibody comprising a polypeptide chain having a variable region from a fully human heavy chain and a variable region from a fully human light chain, and optionally a linker joining the variable heavy and variable light chain regions, wherein the variable heavy chain region comprises at least 95% sequence identity, or at least 96% sequence identity, or at least 97% sequence identity, or at least 98% sequence identity, or at least 99% sequence identity to the amino acid sequence of SEQ ID NO:28, and the variable light chain region comprises at least 95% sequence identity, or at least 96% sequence identity, or at least 97% sequence identity, or at least 98% sequence identity, or at least 99% sequence identity to the amino acid sequence of SEQ ID NO:29. In one embodiment, the linker comprises a peptide linker having the sequence (GGGGS)_{N} (SEQ ID NO: 46) wherein ‘N’ is 1-6. In some embodiments, the linker comprises, consists of, or consists essentially of the amino acid sequence GGGGSGGGGSGGGGS (SEQ ID NO:21).

### Nucleic Acid Molecules, Production Hosts and Methods

The present disclosure provides nucleic acid molecules encoding one or more polypeptides of an antigen-binding protein as disclosed herein that specifically binds the S protein of a coronavirus.

In some embodiments, the present disclosure provides a first nucleic acid molecule encoding a first polypeptide comprising a heavy chain variable region having a heavy chain CDR1 having the amino acid sequence of SEQ ID NO:30, a heavy chain CDR2 region having the amino acid sequence of SEQ ID NO:31, and a heavy chain CDR3 region having the amino acid sequence of SEQ ID NO:32, and a second nucleic acid molecule encoding a second polypeptide comprising the light chain variable region having a light chain CDR1 having the amino acid sequence of SEQ ID NO:33, a light chain CDR2 region having the amino acid sequence of SEQ ID NO:34, and a light chain CDR3 region having the amino acid sequence of SEQ ID NO:35. The first and second nucleic acid molecules can encode a heavy chain and light chain of an antibody, for example. Alternatively, the disclosure provides a single nucleic acid molecule encoding a first polypeptide that comprises a heavy chain variable region having a heavy chain CDR1 having the amino acid sequence of SEQ ID NO:30, a heavy chain CDR2 region having the amino acid sequence of SEQ ID NO:31, and a heavy chain CDR3 region having the amino acid sequence of SEQ ID NO:32, and a second polypeptide comprising the light chain variable region having a light chain CDR1 having the amino acid sequence of SEQ ID NO:33, a light chain CDR2 region having the amino acid sequence of SEQ ID NO:34, and a light chain CDR3 region having the amino acid sequence of SEQ ID NO:35. In a further alternative, the disclosure provides a nucleic acid molecule that encodes a polypeptide having a heavy chain CDR1 having the amino acid sequence of SEQ ID NO:30, a heavy chain CDR2 region having the amino acid sequence of SEQ ID NO:31, and a heavy chain CDR3 region having the amino acid sequence of SEQ ID NO:32, a light chain CDR1 having the amino acid sequence of SEQ ID NO:33, a light chain CDR2 region having the amino acid sequence of SEQ ID NO:34, and a light chain CDR3 region having the amino acid sequence of SEQ ID NO:35. An antigen-binding protein encoded by one or more nucleic acid molecules as provided herein can be, as nonlimiting examples, an IgG or a single chain antibody.

In further embodiments, the present disclosure provides a first nucleic acid molecule encoding a first polypeptide comprising a heavy chain variable region having a heavy chain variable region having at least 95% sequence identity, at least 96% sequence identity, at least 97% sequence identity, at least 98% sequence identity, or at least 99% sequence identity to SEQ ID NO:28, and a second nucleic acid molecule encoding a second polypeptide comprising the light chain variable region having at least 95% sequence identity, at least 96% sequence identity, at least 97% sequence identity, at least 98% sequence identity, or at least 99% sequence identity to SEQ ID NO:29. The first and second nucleic acid molecules can encode a heavy chain and light chain of an antibody, for example. Alternatively, the disclosure provides a single nucleic acid molecule encoding a first polypeptide that comprises a heavy chain variable region having a heavy chain CDR1 having an amino acid having at least 95%, at least 96%, at least 97%, at least 98%, or at least 99% to SEQ ID NO:28, and a second polypeptide comprising a light chain variable region having at least 95%, at least 96%, at least 97%, at least 98%, or at least 99% to SEQ ID NO:29. In further alternatives, the disclosure provides a nucleic acid molecule that encodes a polypeptide having a heavy chain amino acid sequence having at least 95% identity to SEQ ID NO:28 and a light chain amino acid sequence having at least 95% identity to SEQ ID NO:29. An antigen-binding protein encoded by one or more nucleic acid molecules as provided herein can be, as nonlimiting examples, an IgG or a single chain antibody.

A nucleic acid molecule encoding an antigen-binding protein that specifically binds the S protein of a coronavirus as disclosed herein can be an expression vector that includes a promoter operably linked to the protein-encoding sequence. A promoter operably linked to the polypeptide-encoding sequence(s) can be a eukaryotic or prokaryotic promoter but is preferably a eukaryotic promoter that is active in a mammalian cell. The expression vector(s) can direct transcription and/or translation of the transgene in the host cell and can include ribosomal binding sites and/or polyadenylation sites. The polypeptide(s) that include heavy and light chain sequences as disclosed above can be displayed on the surface of the transgenic host cell or secreted into the cell culture medium. In various embodiments, the host cell, or population of host cells, harbor one or more expression vectors that can direct transient introduction of the transgene into the host cells or stable insertion of the transgene into the host cells' genome, where the transgene comprises nucleic acids encoding any of the first and/or second polypeptides described herein.

A production host cell can be a prokaryote, for example, E. coli, or it can be a eukaryote, for example, a single-celled eukaryote (e.g., a yeast or other fungus), a plant cell (e.g., a tobacco or tomato plant cell), a mammalian cell (e.g., a human cell, a monkey cell, a hamster cell, a rat cell, a mouse cell, or an insect cell) or a hybridoma. In one embodiment, a production host cell can be transfected with an expression vector operably linked to a nucleic acid encoding a desired antigen-binding protein thereby generating a transfected/transformed host cell which is cultured under conditions suitable for expression of the antigen-binding protein by the transfected/transformed host cell, and optionally recovering the antibody from the transfected/transformed host cells (e.g., recovery from host cell lysate) or recovery from the culture medium. In one embodiment, production host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In some embodiments, host cells can be lymphoid cells such as Y0, NS0 or Sp20. In some embodiments, a host cell is a mammalian host cell, but is not a human host cell.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

The recombinant DNA can also encode any type of protein tag sequence that may be useful for purifying the protein. Examples of protein tags include but are not limited to a histidine (his) tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into a host cell, e.g., a production host cell, using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is a viral vector).

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, E. coli host cells are suitable for expressing small polypeptides. The protein can then be purified from culture media or cell extracts.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Nucleic acids encoding any of the various polypeptides disclosed herein may be synthesized chemically or using gene synthesis methods (available for example through commercial entities such as Blue Heron, DNA 2.0, GeneWiz, etc.). Codon usage may be selected so as to improve expression in a cell. Such codon usage will depend on the production host cell type. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein can be at least 65% pure, at least 75% pure, at least 85% pure, at least 95% pure, or at least 98% pure. Regardless of the exact numerical value of the purity, the polypeptide is sufficiently pure for use as a pharmaceutical product. Any of the anti-spike protein antibodies, or antigen binding protein thereof, described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, a form of glycosylation can be sialylation, which conjugates one or more sialic acid moieties to the polypeptide. Sialic acid moieties improve solubility and serum half-life while also reducing the possible immunogenicity of the protein. See Rajuetal. Biochemistry 2001 31; 40:8868-76.

In some embodiments, the antibodies and antigen binding proteins described herein can be modified to increase their solubility and/or serum half-life which comprises linking the antibodies and antigen binding proteins to non-proteinaceous polymers. For example, polyethylene glycol (“PEG”), polypropylene glycol, or polyoxyalkylenes can be conjugated to antigen-binding proteins, for example in the manner as set forth in U.S. Pat. Nos. 4,640,835; 4,496,689; 4,301,144; 4,670,417; 4,791,192; or 4,179,337.

The term “polyethylene glycol” or “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C14 alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. Branched PEG molecules are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

### Pharmaceutical Compositions

The present disclosure provides pharmaceutical compositions comprising any of the anti-spike protein neutralizing antibodies or antigen-binding proteins described herein and a pharmaceutically acceptable excipient. In some embodiments, the pharmaceutical compositions comprise an anti-spike protein antibody or antigen binding protein as disclosed herein that is neutralizing with respect to a coronavirus such as SARS-CoV-2, comprising a heavy chain variable region with an amino acid sequence having at least 95%, at least 96%, at least 97%, at least 98%, or at least 99% sequence identity to the amino acid sequence of SEQ ID NO:28 (the heavy chain variable region of antibody S1D7270) and an amino acid sequence having at least 95%, at least 96%, at least 97%, at least 98%, or at least 99% sequence identity to the amino acid sequence of SEQ ID NO:29 (the light chain variable region of antibody S1D7270). Alternatively or in addition, an anti-spike protein antigen binding protein as provided in a pharmaceutical composition as provided herein can be a neutralizing antibody or antigen-binding protein that includes the heavy chain CDR1 sequence of SEQ ID NO:30, the heavy chain CDR2 sequence of SEQ ID NO:31, the heavy chain CDR3 sequence of SEQ ID NO:32, the light chain CDR1 sequence of SEQ ID NO:33, the light chain CDR2 sequence of SEQ ID NO:34, and the light chain CDR3 sequence of SEQ ID NO:35. In various embodiments the pharmaceutical composition includes the IgG antibody S1D7270 or STI-2020 (S1D7270_{LALA}). Any of the pharmaceutical compositions disclosed herein can include, in addition to an antigen binding protein as disclosed herein, one or more additional therapeutic compounds, including one or more additional antibodies, which may be one or more additional neutralizing antibodies.

The pharmaceutical compositions can be produced to be sterile and stable under the conditions of manufacture and storage. The antigen-binding proteins of the composition can be in powder form, for example for reconstitution in the appropriate pharmaceutically acceptable excipient before or at the time of delivery. Alternatively, the antigen-binding proteins can be in solution with an appropriate pharmaceutically acceptable excipient or a pharmaceutically acceptable excipient can be added and/or mixed before or at the time of delivery, for example to provide a unit dosage in injectable or inhalable form. Preferably, the pharmaceutically acceptable excipient used in the present invention is suitable to high drug concentration, can maintain proper fluidity, and, in some embodiments, can delay absorption.

The present disclosure provides therapeutic compositions comprising any of the anti-spike protein antibodies, or an antigen binding portion thereof, described herein and a pharmaceutically-acceptable excipient. Excipients encompass carriers and stabilizers. Examples of pharmaceutically acceptable excipients include for example inert diluents or fillers (e.g., sucrose and sorbitol), lubricating agents, glidants, anti-adhesives (e.g., magnesium stearate, zinc stearate, stearic acid, silicas, hydrogenated vegetable oils, or talc), and, as appropriate to the mode of delivery, adhesives such as mucoadhesives (See, Ugwoke et al., (2005) Advanced Drug Delivery 57:1640-65)). Additional examples of excipients include buffering agents, stabilizing agents, preservatives, non-ionic detergents, anti-oxidants, and isotonifiers.

Therapeutic compositions and methods for preparing them are well known in the art and are found, for example, in Remington, the Science and Practice of Pharmacy, 23rd Edition, Academic Press (2020)). Therapeutic compositions can be formulated for parenteral administration may, and can contain excipients such as, for example, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may in some cases be used to control the release of the antibody or antigen binding protein described herein. Nanoparticulate formulations (e.g., biodegradable nanoparticles, solid lipid nanoparticles, liposomes) may be used to control the biodistribution of the antibody or antigen binding protein. Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the anti-spike protein antibodies disclosed herein (or antigen binding protein) may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding portions thereof) is formulated in the presence of sodium acetate to increase thermal stability.

Any of the anti-spike protein antibodies or antigen-binding proteins disclosed herein may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

In some embodiments, a pharmaceutical composition as provided herein is formulated for nasal delivery. “Nasal delivery” is used herein to refer to deposition of the pharmaceutical composition within one or preferably both nares (nostrils or nasal passages) of a subject; “nasal delivery” and “intranasal delivery” are used interchangeably herein. Nasal delivery may be topical administration, i.e., deposition or application of a liquid, gel, paste, powder, or particles within the nasal passages, for example using a dropper, squeeze bottle, or applicator. In some instances, nasal delivery may be by inhalation, including by means of a dry powder inhaler, metered-dose inhaler, or nebulizer that generates aerosols for delivery of particles or droplets to the lung. Contemplated herein are methods of topical nasal delivery that do not require an inhalation device such as a dry powder inhaler, metered dose inhaler, or nebulizer, although the formulations and methods provided herein are not limited to such methods.

A coronavirus-neutralizing antigen-binding protein as provided herein can be formulated with any suitable excipient(s) for nasal delivery. Reference may be made to standard handbooks, such as for example Remington's Introduction to the Pharmaceutical Sciences, 2nd Ed., Lippincott Williams and Wilkins, USA (2011) or Remington, the Science and Practice of Pharmacy, 23rd Edition, Academic Press (2020). Preferably a neutralizing antibody for nasal administration is formulated as a composition or pharmaceutical composition comprising at least a therapeutically effective amount of a neutralizing antibody, such as a neutralizing antibody disclosed herein, and at least one pharmaceutically acceptable nasal carrier, and optionally one or more additional pharmaceutically acceptable additives and/or agents. A “nasal carrier” as set forth in the present invention is a carrier that is suitable for application through the nasal route, i.e. deposition within the nostril or application to the nasal mucosa. The nasal carrier may be a solid, semi-liquid, or liquid filler, diluent, or encapsulating material, for example. The nasal composition can be provided in a variety of forms, including fluid or semi-liquid or viscous solutions, gels, creams, pastes, powders, microspheres, and films for direct application to the nasal mucosa including application as liquid drops into the nasal passage. The nasal carrier should be “pharmaceutically acceptable” in the sense of being compatible with the other ingredients of the formulation and not eliciting an unacceptable deleterious effect in the subject. Preferred nasal carriers are those which maintain the solubility of the neutralizing antibody (when applied as a liquid or in a gel or paste) and those that improve the contact of the pharmaceutical composition with the nasal mucus or nasal mucosa. A carrier provided in the composition may also facilitate the diffusion of the antibody from the composition to the nasal mucosa and/or may prolong the nasal residence time of the composition allowing dissemination to the lungs via respiration, for example.

Suitable nasal carriers are known to those skilled in the art of pharmacology. Such carriers may be used in suitable amounts known per se, which will be clear to the skilled person based on the disclosure and art cited herein. A carrier used in a composition for intranasal delivery can be a liquid or solvent or dispersion medium containing, for example, water, ethanol, a polyol (for example, glycerol, propylene glycol, polyethylene glycol, and the like), and suitable mixtures thereof. Other preferred liquid carriers are aqueous saline, e.g. physiological saline, or an aqueous buffer, e.g. a phosphate/citric acid buffer. Further components of the composition, which can be soluble, in semi-solid or gelatinous form, sparingly soluble, or in solid form can include, without limitation, additional salts, one or more sugars, or polymers, for example, mannitol, lactose, starch, magnesium stearate, sodium saccharin, talcum, cellulose, glucose, sucrose, magnesium carbonate, and the like. Further carriers include polyacrylates, sodium carboxy methyl cellulose, starches and their derivatives, alginic acid and salts, hyaluronic acid and salts, pectic acid and salts, gelatin and its derivatives, gums, polylactic acid and its copolymers, polyvinyl acetate, celluloses and their derivatives, coated celluloses, crosslinked dextrans, polylactic acid and its copolymers, and polyvinyl acetate. Non-limiting examples of other solid nasal carriers are described in U.S. Pat. No. 5,578,5674, WO 04/093917 and WO 05/120551.

In some embodiments, the neutralizing antibody may be released from the composition by diffusion or by disintegration of the nasal carrier. In some circumstances, the neutralizing antibody is dispersed in microcapsules (microspheres) or nanocapsules (nanospheres) prepared from a suitable polymer, e.g., isobutyl 2-cyanoacrylate (see, e.g., Michael et al., J. Pharmacy Pharmacol. 1991; 43: 1-5). These particular microspheres not only demonstrate mucoadhesion properties, but also protect against enzymatic degradation. They may further allow manipulation of the rate of release of the neutralizing antibody or antibodies to provide sustained delivery and biological activity over a protracted time (Morimoto et al., Eur. J. Pharm. Sci. 2001 May; 13(2): 179-85).

The pharmaceutical composition can include a bioadhesive nasal carrier, e.g., a compound that adheres to the nasal mucosa by chemical or physical binding such as Van der Waals interaction, ionic interaction, hydrogen bonding or by polymer chain entanglement. The adhesion may be to the epithelial (cellular) surface or to the mucus overlying the surface (a mucoadhesive). These compounds promote binding of drugs to biological material in the nasal cavity, thereby extending residence time and allowing delivery to the lungs through respiration. Examples of bioadhesive or mucoadhesive materials include, without limitation, carhopol, cellulose and cellulose derivatives (e.g., hydroxypropyl methylcellulose, hydroxypropylcellulose) or cellulose-containing compounds, coated cellulose (e.g., microcrystalline cellulose coated with glycerol monooleate) starch, dextran, and chitosan (See, for example, Ilium, Bioadhesive formulations for nasal peptide delivery. In: E Mathiowitz, D E Chickering III, C Lehr, eds. Bioadhesive Drug Delivery Systems. New York: Marcel Dekker, 1999: 507-541; EP 0490806; and WO 96/03142).

To further enhance mucosal delivery of a neutralizing antibody as provided herein, formulations comprising neutralizing antibody may also contain a hydrophilic low molecular weight compound as a nasal carrier, base, or excipient. Such hydrophilic low molecular weight compounds provide a passage medium through which a water-soluble active agent, such as the neutralizing antibody of the invention, may diffuse. Exemplary hydrophilic low molecular weight compound are disclosed in WO 05/120551 and include polyol compounds, such as oligo-, di- and monosaccarides such as sucrose, mannitol, lactose, L-arabinose, D-erytrose, D-ribose, D-xylose, D-mannose, D-galactose, lactulose, cellobiose, gentibiose, glycerin, and polyethylene glycol. Other non-limiting examples of hydrophilic low molecular weight compounds useful as such carriers or bases include N-methylpyrrolidone, and alcohols (e.g. oligovinyl alcohol, ethanol, ethylene glycol, propylene glycol, etc.). These hydrophilic low molecular weight compounds can be used alone or in combination with one another or with other active or inactive components of the intranasal formulation.

A carrier in a nasal formulation may further contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, stabilizers, tonicity adjustors, wetting agents or other biocompatible materials. A tabulation of ingredients listed by the above categories can be found in the U.S. Pharmacopeia National Formulary, pp. 1857-1859, 1990. Examples of pharmaceutically acceptable preservatives include benzalkonium chloride, an alkyl p-hydroxybenzoate (paraben) such as methyl p-hydroxybenzoate and propyl p-hydroxybenzoate, or sodium methylmercurithiosalicylate (Thiomersal). Further non-limiting examples of pharmaceutically acceptable preservatives are described in U.S. Pat. No. 5,759,565, US 20100129354, and WO 04/093917. Examples of pharmaceutically acceptable antioxidants include alkali metal sulfites, alkali metal bisulfites, alkali metal pyrosulfites, sodium thiosulfate, thiodipropionic acid, cysteine in free or salt form (such as cysteine hydrochloride), ascorbic acid, citraconic acid, propyl or ethyl gallate, nordihydroguaiaretic acid, butylated hydroxyanisole or -toluene, and tocol. Further non-limiting examples of pharmaceutically acceptable antioxidants are provided in US 20100129354, WO 04/093917, and WO 05/120551.

The desired viscosity for the compositions of the invention will depend on the particular form for administration, e.g. whether administration is to be by nasal drops or nasal spray. For example, for nasal drops an appropriate viscosity may be from about 2 to about 40×10⁻³ Pa s, and for nasal sprays the viscosity may be less than 2×10⁻³ Pa s, e.g. from 1 to 2×10⁻³ Pa s. Such values are exemplary only, and acceptable viscosities for a therapeutic or prophylactic neutralizing antibody composition can be determined empirically. Examples of pharmaceutically acceptable compounds for enhancing viscosity include, for example, methylcellulose, hydroxymethylcellulose, hydroxypropylmethylcellulose, (which can also serve as mucoadhesives) sucrose, PVA, PVP, polyacrylic acid, or natural polymers. Further non-limiting examples of viscosity builders are described in U.S. Pat. No. 5,578,567.

Examples of pharmaceutically acceptable stabilizers include albumin, e.g. human serum albumin, aprotinin or S-aminocaproic acid. In some instances, the activity or physical stability of proteins can also be enhanced by various additives to aqueous solutions of the neutralizing antibody or antibodies. For example, additives, such as polyols (including sugars), amino acids, and various salts may be used. Further non-limiting examples of stabilizers are provided in US 20100129354. Examples of pharmaceutically acceptable tonicity adjustors include nasally acceptable sugars, e.g. glucose, mannitol, sorbitol, ribose, mannose, arabinose, xylose or another aldose or glucosamine. Further non-limiting examples of tonicity adjustors are provided in US20100129354. Such additives may be used in suitable amounts as known in the art and as can be determined by the skilled person based on the disclosure and art, including art cited herein.

Enzyme inhibitors may also be added to the composition for nasal delivery to reduce the activity of any hydrolytic enzymes in the nasal mucosa that can potentially degrade the neutralizing antibody or other components of the pharmaceutical composition. Enzyme inhibitors that can reduce degradative activities for use within the invention can be selected from a wide range of non-protein inhibitors that vary in their degree of potency and toxicity (see, e.g., L. Stryer, Biochemistry, WI-1: Freeman and Company, NY, N.Y., 1988). Non-limiting examples include amastatin and bestatin (O'Hagan et al., Pharm. Res. 1990, 7: 772-776). Various classes of enzyme inhibitors that may be considered are extensively described and exemplified in WO 05/120551. Another means to inhibit degradation is pegylation with PEG molecules, preferably low molecular weight PEG molecules (e.g. 2 kDa; Lee et al., Calcif Tissue Int. 2003, 73: 545-549).

When the nasal carrier is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7. Liquid compositions of the invention further preferably have a mildly acid pH, e.g. from about pH 3 to about pH 6.5, from about pH 3.5 to about pH 6.5, or preferably from about pH 4.5 to about pH 6.5 to minimize nasal irritation (Betel et al. Adv. Drug Delivery Rev. 1998; 29: 89-116). The required degree of acidity may conveniently be achieved, e.g by the addition of a buffering agent, e.g. a mixture of citric acid and disodium hydrogen phosphate, or an acid such as HCl or another appropriate mineral or an organic acid, e.g. phosphoric acid. Solid compositions may also comprise a buffering agent when they are prepared by lyophilization of a liquid composition buffered to a pH value as indicated above. Non-limiting examples of pharmaceutically acceptable buffering agents are provided in US20100129354, WO 04/093917, and WO 05/120551. In some embodiments provided herein, a composition for topical nasal delivery that includes a neutralizing antibody as provided herein, such as the STI-2020 antibody, comprises: a buffering agent such as histidine, for example, from 5-100 mM histidine (e.g., from about 10-50 mM histidine), from about 200 to about 300 mM sucrose, and from about 0.1% to 0.5%, for example from about 0.2% to about 0.3% hydroxypropyl methyl cellulose (HPMC), and from about 0.01% to about 0.2% polysorbate 80, at a pH of about 5.5-6, such as about pH 5.8. For example, a formulation for nasal delivery can include 20 mM Histidine, 240 mM Sucrose, 0.3% Hydroxypropyl methyl cellulose (HPMC), and 0.05% Polysorbate 80, at a pH of about 5.8. The concentration of antibody can be, for example, from about 1 mg/mL to about 200 mg/mL, from about 2 mg/mL to about 100 mg/mL, or from about 5 mg/mL to about 80 mg/mL, or from about 10 mg/mL to about 50 mg/mL.

Alternatively, a neutralizing antibody, such as a neutralizing antibody provided herein may be formulated for intranasal delivery in the form of a powder (such as a freeze-dried or micronised powder) or mist; for example with a particle size within the ranges indicated herein. Compounds may also be included in a pharmaceutical composition for intranasal delivery to reduce or prevent aggregation of the neutralizing antigen-binding protein. Aggregation inhibitory agents include, for example, polymers such as polyethylene glycol, dextran, diethylaminoethyl dextran, and carboxymethyl cellulose, which significantly increase the stability and reduce the solid-phase aggregation of peptides and proteins. Certain additives, in particular sugars and other polyols, also impart significant physical stability to dry, e.g., lyophilized proteins. These additives can also be used within the invention to protect the proteins against aggregation not only during lyophilization but also during storage in the dry state. For example, sucrose and Ficoll 70 (a polymer with sucrose units) exhibit significant protection against peptide or protein aggregation during solid-phase incubation under various conditions. These additives may also enhance the stability of solid proteins embedded within polymer matrices. Yet additional additives, for example sucrose, stabilize proteins against solid-state aggregation in humid atmospheres at elevated temperatures, as may occur in certain sustained-release formulations of the invention. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time. A wide non-limiting range of suitable methods and anti-aggregation agents are available for incorporation within the compositions of the invention such as disclosed in WO 05/120551, Breslow et al. (J. Am. Chem. Soc. 1996; 118: 11678-11681), Breslow et al. (PNAS USA 1997; 94: 11156-11158), Breslow et al. (Tetrahedron Lett. 1998; 2887-2890), Zutshi et al. (Curr. Opin. Chem. Biol. 1998; 2: 62-66), Daugherty et al. (J. Am. Chem. Soc. 1999; 121: 4325-4333), Zutshi et al. (J. Am. Chem. Soc. 1997; 119: 4841-4845), Ghosh et al. (Chem. Biol. 1997; 5: 439-445), Hamuro et al. (Angew. Chem. Int. Ed. Engl. 1997; 36: 2680-2683), Alberg et al., Science 1993; 262: 248-250), Tauton et al. (J. Am. Chem. Soc. 1996; 118: 10412-10422), Park et al. (J. Am. Chem. Soc. 1999; 121: 8-13), Prasanna et al. (Biochemistry 1998; 37:6883-6893), Tiley et al. (J. Am. Chem. Soc. 1997; 119: 7589-7590), Judice et al. (PNAS USA 1997; 94: 13426-13430), Fan et al. (J. Am. Chem. Soc. 1998; 120: 8893-8894), Gamboni et al. (Biochemistry 1998; 37: 12189-12194).

Where a carrier is included in a solid nasal composition, the particle size of the components including the carriers, of the invention may be from 5 to 500μ, preferably from 10 to 250μ, more preferably from 20 to 200μ. For example, the average particle size may be in the range of 50 to 100μ.

A therapeutic or prophylactic composition for nasal delivery as provided herein can be provided in a kit comprising an anti-S antigen binding protein as disclosed herein. In one embodiment, the kit comprises an antigen binding protein that specifically binds the S protein of a coronavirus as disclosed herein, such as an anti-S protein comprising a heavy chain variable region having at least 95% identity, or at least 96% identity, or at least 97% identity, or at least 98% identity, or at least 99% identity to SEQ ID NO:28 and a light chain variable region having at least 95% identity, or at least 96% identity, or at least 97% identity, or at least 98% identity, or at least 99% identity to SEQ ID NO:29. The kit can further include one or more sterile pharmaceutically acceptable solutions for resuspension or dilution of the antibody, one or more additional pharmaceutical formulations, which may be, as nonlimiting examples, any of an additional antibody, an analgesic, an antibiotic, an anti-inflammatory drug, a bronchodilator, or an antiviral drug. The kit can be used for treating a subject having a coronavirus infection, or for providing prophylaxis against infection with a coronavirus. The components of the kit of can be provided in suitable containers and labeled for diagnosis, prophylaxis and/or treatment of coronavirus infection. The above-mentioned components may be stored in unit or multi-dose containers, for example, sealed ampules, vials, bottles, syringes, and test tubes, as an aqueous, preferably sterile, solution or as a lyophilized, preferably sterile, formulation for reconstitution. The containers may be formed from a variety of materials such as glass or plastic and may have a sterile access port (for example, the container may be an intravenous solution bag or a vial having a stopper pierceable by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. Associated with the kits can be instructions customarily included in commercial packages of therapeutic, prophylactic or diagnostic products, that contain information about, for example, the indications, usage, dosage, manufacture, administration, contraindications and/or warnings concerning the use of such therapeutic, prophylactic or diagnostic products.

### Methods of Treatment

The present disclosure provides methods for treating a subject having a coronavirus infection or suspected of having a coronavirus infection, the method comprising: administering to the subject an effective amount of a therapeutic composition comprising a neutralizing anti-S1 antigen-binding protein as described herein, e.g., an antibody as described herein. The subject can be a human subject or an animal. The subject may be, for example, a subject who has tested positive for the coronavirus, a subject who has had contact with another individual or animal that has tested positive for coronavirus, a subject who is likely to come into contact with another individual or animal infected with coronavirus, and/or can be a subject exhibiting symptoms associated with coronavirus infection. The antigen-binding protein or antibody is preferably a fully human neutralizing antigen-binding protein or antibody and may be a fully human neutralizing antibody having one or more mutations in the Fc region that result in reduced Fc effector function. In some embodiments, the subject is infected with or suspected of being infected with HCoV-NL63, SARS-CoV, or SARS-CoV-2, for example, the subject may be a human subject infected with or suspected of being infected with SARS-CoV-2.

The subject may be infected with or suspected of being infected with a variant of SARS-Cov-2, for example, the variant B.1.1.7 or B.1.351 or another variant, for example, a variant encoding an S protein having at least 85%, at least 90%, at least 95%, at least 96%, at least 97%, at least 98%, or at least 99% identity to SEQ ID NO:1 or SEQ ID NO:2 or encoding an S protein having an S1 domain having at least 85%, at least 90%, at least 95%, at least 96%, at least 97%, at least 98%, or at least 99% identity to SEQ ID NO:4.

The present disclosure also provides methods for preventing a coronavirus infection in a subject, the method comprising: administering to a subject at risk of becoming infected with a coronavirus an effective amount of a therapeutic composition comprising an anti-S1 antigen-binding protein as described herein, which may be an antibody or antibody fragment as described herein. The subject can be an animal or a human subject. The subject may be a health care worker; a first responder; a human services worker; a transportation worker; a delivery person; a restaurant, food services, airline, or hospitality industry worker; a worker in a food processing or meat-packing plant; or a worker in a factory, assembly plant, or warehouse. The subject may be an incarcerated subject in a jail or prison. The subject may be a person living or working in a nursing home, rehabilitation center, or an assisted living facility. The subject may be a person living or working at a college, university, or school. The subject may live in an area with a high rate of increase of people testing positive for the coronavirus. The antigen-binding protein or antibody is preferably a fully human neutralizing antigen-binding protein or antibody as disclosed herein and may be a fully human neutralizing antibody such as an IgG having one or more mutations in the Fc region that result in reduced Fc effector function. In some embodiments, the subject is at risk of being infected with HCoV-NL63, SARS-CoV, or SARS-CoV-2, for example, the subject may be a human subject at risk of becoming infected with SARS-CoV-2.

The subject may be at risk of being infected with a variant of SARS-Cov-2, for example, the variant B.1.1.7 or B.1.351 or another variant, for example, a variant encoding an S protein having at least 85%, at least 90%, at least 95%, at least 96%, at least 97%, at least 98%, or at least 99% identity to SEQ ID NO:1 or SEQ ID NO:2 or encoding an S protein having an S1 domain having at least 85%, at least 90%, at least 95%, at least 96%, at least 97%, at least 98%, or at least 99% identity to SEQ ID NO:4.

In some embodiments, administration of the antigen binding protein that specifically binds the S protein of a coronavirus, such as SARS-CoV-2, can be by oral delivery. Oral dosage forms can be formulated for example as tablets, troches, lozenges, aqueous or oily suspensions, dispersible powders or granules, emulsions, hard capsules, soft gelatin capsules, syrups or elixirs, pills, dragees, liquids, gels, or slurries. These formulations can include pharmaceutically excipients including, but not limited to, inert diluents such as calcium carbonate, sodium carbonate, lactose, calcium phosphate or sodium phosphate; granulating and disintegrating agents such as corn starch or alginic acid; binding agents such as starch, gelatin or acacia; lubricating agents such as calcium stearate, glyceryl behenate, hydrogenated vegetable oils, magnesium stearate, mineral oil, polyethylene glycol, sodium stearyl, fumarate, stearic acid, talc, zinc stearate; preservatives such as n-propyl-p-hydroxybenzoate; coloring, flavoring or sweetening agents such as sucrose, saccharine, glycerol, propylene glycol or sorbitol; vegetable oils such as arachis oil, olive oil, sesame oil or coconut oil; mineral oils such as liquid paraffin; wetting agents such as benzalkonium chloride, docusate sodium, lecithin, poloxamer, sodium lauryl sulfate, sorbitan esters; and thickening agents such as agar, alginic acid, beeswax, carboxymethyl cellulose calcium, carageenan, dextrin or gelatin.

Alternatively, administration of the antigen binding protein, e.g., antibody composition, can be by injection or intravenous or intra-arterial delivery, and may be, for example, by epidermal, intradermal, subcutaneous, intramuscular, intraperitoneal, intrapleural, intra-abdominal, or intracavitary delivery. Delivery by injection can be by “push” or bolus injection that may be intravenous or intraarterial, or intramuscular, intradermal, subcutaneous, or epidermal. Alternatively intravenous or intra-arterial delivery, for example, can be by infusion. Formulations for parenteral administration can be inter alia in the form of aqueous or nonaqueous isotonic sterile non-toxic injection or infusion solutions or suspensions. Preferred parenteral administration routes include intravenous, intra-arterial, intraperitoneal, epidural, and intramuscular injection or infusion. The solutions or suspensions may comprise agents that are non-toxic to recipients at the dosages and concentrations employed such as 1,3-butanediol, Ringer's solution, Hank's solution, isotonic sodium chloride solution, oils such as synthetic mono- or diglycerides or fatty acids such as oleic acid, local anesthetic agents, preservatives, buffers, viscosity or solubility increasing agents, water-soluble antioxidants such as ascorbic acid, cysteine hydrochloride, sodium bisulfate, sodium metabisulfite, sodium sulfite and the like, oil-soluble antioxidants such as ascorbyl palmitate, butylated hydroxyanisole (BHA), butylated hydroxytoluene (BHT), lecithin, propyl gallate, alpha-tocopherol, and the like, and metal chelating agents, such as citric acid, ethylenediamine tetraacetic acid (EDTA), sorbitol, tartaric acid, phosphoric acid, etc. The antibody concentration can be determined using guidance from animal studies and clinical studies, and can range from, as nonlimiting examples, about 1 μg/mL to about 10 g/mL, or from about 10 μg/ml to about 5 g/mL, for example, from about 10 μg/ml to about 5 g/mL, from about 20 μg/ml to about 2 g/mL, from about 100 μg/ml to about 2 g/mL, for example, from about 1 mg/mL to about 2 g/mL, or from about 1 mg/mL to about 1 g/mL, or from about 2 mg/mL to about 500 mg/mL, from about 2 mg/mL to about 200 mg/mL, from about 2 mg/mL to about 100 mg/mL, from about 2 mg/mL to about 50 mg/mL, from about 5 mg/mL to about 200 mg/mL, from about 5 mg/mL to about 100 mg/mL, or from about 5 mg/mL to about 50 mg/mL. A total single dose can be from about 1 μg to about 10 g, or from about 10 μg to about 5 mg/mL, for example, from about 10 μg to about 2 g, from about 20 μg to about 500 mg, from about 100 μg to about 200 mg, for example, from about 1 mg to about 1 g, or from about 2 mg to about 500 mg, or from about 2 mg to about 200 mg, from about 2 mg/mL to about 100 mg, from about 5 mg to about 200 mg, from about 5 mg to about 100 mg.

A subject can be treated with a single intravenous dose or with multiple intravenous doses, for example, over a period of hours or days.

In various embodiments, an antibody (or antibody fragment) that specifically binds an epitope of a coronavirus S1 subunit, such as any of the neutralizing antibodies disclosed herein, can be incorporated into a pharmaceutical composition suitable for pulmonary administration to a subject. For example, an antibody as provided herein antibody that specifically binds an epitope of a coronavirus S1 subunit can be formulated into a liquid pharmaceutical composition that includes a pharmaceutical excipient, where pharmaceutical composition is suitable for inhalation by a subject. The neutralizing antibody can comprise a heavy chain variable region having at least 95%, at least 96%, at least 97%, at least 98%, or at least 99% sequence identity to the amino acid sequence of SEQ ID NO:28 and can comprise a light chain variable region having 95%, at least 96%, at least 97%, at least 98%, or at least 99% sequence identity to the amino acid sequence of SEQ ID NO:29. In some embodiments, the neutralizing antibody comprises a heavy chain variable region comprising the sequence of SEQ ID NO:28 or a sequence having at least 99% identity to SEQ ID NO:28. In various embodiments the antigen-binding proteins have a heavy chain CDR1 sequence of SEQ ID NO:30, a heavy chain CDR2 sequence of SEQ ID NO:31, a heavy chain CDR3 sequence of SEQ ID NO:32, a light chain CDR1 sequence of SEQ ID NO:33, a light chain CDR2 sequence of SEQ ID NO:34, and a light chain CDR3 sequence of SEQ ID NO:35. In some embodiments, the neutralizing antibody comprises a light chain variable region comprising the sequence of SEQ ID NO:29 or a sequence having at least 99% identity to SEQ ID NO:29. The neutralizing antibody can be an immunoglobulin molecule than optionally includes one or more mutations in the Fc region, for example one or both of a LALA mutation and a YTE mutation as described hereinabove.

A liquid composition that comprises an anti-neutralizing antibody that specifically binds an epitope of a coronavirus S protein as disclosed herein formulated for pulmonary administration can comprise a neutralizing antibody formulated into a solution or suspension, e.g., an isotonic saline solution, which is optionally buffered, at an appropriate concentration for pulmonary administration as an aerosol, mist, or vapor. Preferably, a solution or suspension that includes the neutralizing antibody is isotonic with respect to pulmonary fluids and of about the same pH, for example, has a pH of from about pH 4.0 to about pH 8.5 or from pH 5.5 to pH 7.8, or, for example, from about 7.0 to about 8.2. Suitable buffering agents that can be present in a liquid pharmaceutical composition for pulmonary delivery include, but are not limited to, citrate buffer, phosphate buffer, and succinate buffer. Alternatively or in addition, imidazole, histidine, or another compound that maintains pH in the range of about pH 4.0 to about 8.5 can be used. For example, Ringer's solution, isotonic sodium chloride, and phosphate buffered saline may be used. One of skill in the art can determine an appropriate saline content and pH for an aqueous solution for pulmonary administration.

Additional compounds that may be present in a liquid formulation for pulmonary delivery include, without limitation, sugars, sugar alcohols, alcohols (e.g., benzyl alcohol), polyols, amino acids, salts, polymers, surfactants, and preservatives (e.g., ethyl or n-propyl p-hydroxybenzoate). Other possible ingredients include suspending agents such as cellulose derivatives, sodium alginate, polyvinyl-pyrrolidone, and gum tragacanth, and a wetting agent such as lecithin. The compositions can include any of a variety of compounds to aid in solubility, stability, or delivery, where the added compounds do not negatively affect the coronavirus S-protein binding activity of the neutralizing antibody.

For example, a liquid pharmaceutical composition for pulmonary delivery of an S1-binding neutralizing antibody as provided herein may include an excipient or stabilizer including but not limited to a sugar, alcohol, sugar alcohol, or an amino acid. Preferred sugars include sucrose, trehalose, raffinose, stachyose, sorbitol, glucose, lactose, dextrose, or any combination thereof. A sugar can optionally be present in the range of about 0% to about 9.0% (w/v), preferably about 0.5% to about 5.0%, for example about 1.0%. An amino acid, for example, can be optionally be present in the range of about 0% to about 1.0% (w/v), preferably about 0.3% to about 0.7%, for example about 0.5%.

A buffering agent such as phosphate, citrate, succinate, histidine, imidazole, or Tris can also optionally be present in the liquid neutralizing antibody formulation. EDTA may be present as a stabilizer. Any of various surfactants may also be present, such as for example, polyoxyethylene sorbitol esters such as polysorbate 80 (Tween 80) and polysorbate 20 (Tween 20); polyoxypropylene-polyoxyethylene esters such as Poloxamer 188; polyoxyethylene alcohols such as Brij 35; a mixture of polysorbate surfactants with phospholipids (such as phosphatidylcholine and derivatives), dimyristoylglycerol and other members of the phospholipid glycerol series; lysophosphatidylcholine and derivatives thereof, mixtures of polysorbates with lysolecithin or cholesterol; bile salts and their derivatives such as sodium cholate, sodium deoxycholate, sodium glycodeoxycholate, sodium taurocholate, etc. Additional examples of suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty acids, sorbitan trioleate (Span 85), glycocholate, surfactin, poloxomers, sorbitan fatty acid esters, tyloxapol, phospholipids, and alkylated sugars.

Such pharmaceutical compositions may be administered for example, as a propellant-free inhalable solution comprising a soluble S protein-binding neutralizing antibody and may be administered to the subject via a nebulizer. Other suitable preparations include, but are not limited to, mist, vapor, or spray preparations so long as the particles comprising the protein composition are delivered in a size range consistent with that described for the delivery device.

Therapeutic compositions are preferably sterile and stable under the conditions of manufacture and storage. The formulation can be formulated as a solution, microemulsion, dispersion, or suspension. Sterile inhalable solutions can be prepared by incorporating the active compound (i.e., a soluble S1-binding neutralizing antibody as provided herein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. Fluidity of a solution can be maintained, for example, by the use of a coating such as lecithin, by the maintenance of the required particle size in the case of dispersion, and/or by the use of surfactants.

The concentration of S protein-binding neutralizing antibody in a liquid formulation for pulmonary delivery can range for example, from about 1 μg per ml to about 500 mg per ml, and may be in the range of, for example, from about 10 μg per ml to about 200 mg per ml, or from about 20 μg per ml to about 100 mg per ml, although these ranges are not limiting.

Also provided herein are methods of administering a pharmaceutical composition that comprises one or more nucleic acid molecules that encode a neutralizing antigen binding protein as provided herein. The nucleic acid molecule(s) can comprise RNA or DNA. In some embodiments the nucleic acid molecule(s) is a plasmid that includes expression control sequences such as, but not limited to, one or more promoters, enhancers, or polyA addition sequences. The plasmid can be a nanoplasmid that has minimal bacterial sequences (e.g., U.S. Pat. No. 9,550,998, incorporated herein by reference). The nucleic acid molecule can be administered by injection, e.g., intramuscular or intradermal injection, or by electroporation or needleless injection. The nucleic acid molecule(s) can be formulated with one or more compounds that promote the transfer of nucleic acid molecules into cells, such as but not limited to any disclosed herein. The subject being treated can be a subject being treated for a coronavirus infection or the method can be a method of preventing a coronavirus infection. Dosing may optionally be repeated, for example, over a period of weeks or months.

A pharmaceutical composition that comprises one or more nucleic acid molecules encoding a neutralizing antigen binding protein as provided herein, can further include one or more additional therapeutic compounds. A pharmaceutical composition that comprises one or more nucleic acid molecules encoding a neutralizing antigen binding protein as provided herein, can further include one or more additional nucleic acid molecules, for example, one or more additional nucleic acid molecules that encode one or more additional neutralizing antibodies.

A pharmaceutical composition that includes a nucleic acid molecule encoding an antigen-binding protein as provided herein can include one or more compounds to enhance nucleic acid delivery into cells. (e.g., linear polaxamers including Synperonics®, Pluronics®, and Kolliphor®, and X-shaped polaxamines such as Tetronics®). In various embodiments formulations can include one or more poloxamers, for example, one or more of poloxamer 181, poloxamer 188, and poloxamer 407 at concentrations ranging from 0.1% to 10% or 0.5-5% of the composition. For example, a formulation that includes a nucleic acid molecule can include SP1017 (a composition of poloxamer 181 and poloxamer 407) at a concentration ranging from 0.1% to 10% of the overall composition.

Alternatively or in addition to a poloxamer, a pharmaceutical nucleic acid formulation can include a poloxamine (e.g., T/908 or T/1301 at concentrations ranging from 0.1 to 10% or 0.5-5% of the formulation; additional examples of poloxamines (Tetronics) that may be considered are disclosed in Alvarez-Lorenzo et al. (2010) Frontiers in Bioscience 3:424-440); a cationic lipid such as 1,2-dioleyloxy-3-dimethylaminopropane (DODMA, at concentrations ranging from 0.01% to 1%); alginate, for example, at concentrations ranging from 0.01% to 1%; or DSPE-PEG2000 (1,2-distearoyl-sn-glycero-3-phosphoethanolamine-N-[amino(polyethylene glycol)-2000]) at concentrations ranging from 0.01% to 1%. In some examples a nucleic acid formulation can include poly-beta-amino esters, for example, at a concentration of from about 0.01% to 1%.

A nucleic acid formulation can also include a buffer such as, for example, 0.1%-5% tyrode's solution (137 mM NaCl, 2.7 mM KCl, 1.0 mM MgCl₂, 1.8 mM CaCl₂) 20 mM Hepes, and 5.6 mM glucose, pH 7.1), tris-buffered saline (TBS), or PBS.

The present disclosure provides kits comprising any of the neutralizing antibodies having increased in vivo serum half-life and/or reduced effector function described herein, or antigen binding protein thereof, in an admixture with a pharmaceutically-acceptable excipient, where the heavy chain variable region amino acid sequence has at least 95% sequence identity, at least 96% sequence identity, at least 97% sequence identity, at least 98% sequence identity, or at least 99% sequence identity to SEQ ID NO:28 and the light chain variable region amino acid sequence has at least 95% sequence identity, at least 96% sequence identity, at least 97% sequence identity, at least 98% sequence identity, or at least 99% sequence identity to SEQ ID NO:29. The antibody can be provided as a dry powder, formulated with one or more suitable excipients, or as a liquid formulation. The kit can further include: solutions for resuspension or dilution of the antibody and a means for dispensing the pharmaceutical composition comprising the antibody into a nebulizer or metered dose inhaler. The kit may in some embodiments include a metered dose inhaler. In one embodiment, the kit can be used for treating a subject having a coronavirus-associated infection or disease.

The present disclosure provides methods for treating a subject having a coronavirus infection, the method comprising: administering to the subject an effective amount of a therapeutic composition comprising a neutralizing antibody as provided herein by inhalation. The present disclosure also provides methods of preventing infection with a coronavirus such as SARS-CoV or SARS-Cov-2. The method includes administering to a subject at risk of becoming infected with a coronavirus such as SARS-CoV or SARS-Cov-2 an effective amount of a neutralizing antibody as disclosed herein, for example in a pharmaceutical formulation as disclosed herein, to the subject. Administration is by bronchial or pulmonary delivery, such as by inhalation.

In various embodiments of treatment the composition is administered by pulmonary delivery, for example by oral inhalation. Pulmonary delivery can use any delivery device that can deliver a liquid (e.g., droplets) to the lungs, e.g., can deliver aerosols comprising a therapeutic composition such as a liquid pharmaceutical composition comprising a neutralizing antibody as provided herein to the lungs.

The subject can be a human subject and can be a patient testing positive for a coronavirus such as hCov-NL63, SARS-CoV, or SARS-CoV-2. In some embodiments the subject is a subject testing positive for SARS-CoV-2 or exhibiting symptoms of infection with SARS-CoV-2. In some embodiments, the neutralizing antibody(ies) can be administered to the subject in combination with at least one anti-viral agent and/or at least one viral entry inhibitor. One skilled in the art can routinely select an appropriate anti-viral agent or viral entry inhibitor to be administered with a neutralizing antibody. In one embodiment, the anti-viral agent and/or the viral entry inhibitor can be administered prior to, during, or after, administration of the neutralizing antibody.

Administration of a pharmaceutical formulation that include a neutralizing S1-binding antibody by pulmonary delivery via inhalation can use any device that provides respiratable droplets or particles that are able to reach the lungs by inhalation, preferably by oral inhalation. For example, pulmonary delivery can be by means of a delivery device such as but not limited to a nebulizer or a metered dose inhaler.

The formulations of the invention may include a “therapeutically effective amount” of a neutralizing S1-binding antibody as provided herein. A “therapeutically effective amount” refers to an amount effective, at dosages and for periods of time necessary, to achieve the desired therapeutic result. A therapeutically effective amount of the neutralizing antibody may vary according to factors such as the viral load, disease state, age, sex, and weight of the individual, and the ability of the neutralizing S1-binding antibody to elicit a desired response in the individual. A therapeutically effective amount is also one in which any toxic or detrimental effects of the neutralizing S1-binding antibody are outweighed by the therapeutically beneficial effects.

Toxicity and therapeutic efficacy of the active ingredients described herein can be determined using standard pharmaceutical procedures including in vitro, in cell cultures, and experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually, for example, to provide serum and cell levels of the active ingredient which are sufficient to induce or suppress the biological effect (minimal effective concentration, MEC). Dosages necessary to achieve the MEC will depend on individual characteristics including the severity of the viral infection and related pathologies, the condition of the patient, and the judgment of the physician. Depending on the severity and responsiveness of the condition to be treated, dosing can be of a single or a plurality of administrations, with course of treatment lasting from several days to several weeks or until cure is effected or diminution of the disease state is achieved.

The delivery device can deliver, in a single dose or in multiple doses, a pharmaceutically effective amount of the composition to the subject's lungs by pulmonary inhalation. Devices suitable for pulmonary delivery of a dry powder form of a protein composition as a nonaqueous suspension are commercially available. Examples of such devices include the Ventolin metered-dose inhaler (Glaxo Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference. An aerosol propellant used in an aerosol delivery device may be any conventional material employed for this purpose, such as a chlorofluorocarbon, a hydrochloro-fluorocarbon, a hydrofluorocarbon, or a hydrocarbon, including trichlorofluoromethane, dichlorodifluoro-methane, dichlorotetrafluoromethane, dichlorodifluoro-methane, dichlorotetrafluoroethanol, and 1,1,1,2-tetra-fluoroethane, or combinations thereof.

Where the solid or dry powder form of the formulation is to be delivered as a dry powder form, a dry powder inhaler or other appropriate delivery device is preferably used. The dry powder form of the formulation is preferably prepared as a dry powder aerosol by dispersion in a flowing air or other physiologically acceptable gas stream. For example, the delivery device can be any of dispenser is of a type selected from the group consisting of a reservoir dry powder inhaler (RDPI), a multi-dose dry powder inhaler (MDPI), and a metered dose inhaler (MDI).

Liquid aerosol delivery by nebulizer is another form of pulmonary drug delivery that can be employed. Nebulizers as they are generally more effective for delivery to the deep lung and may be preferred for delivering protein therapeutics in active form. Nebulizers create liquid aerosols, which are forced from a small orifice at high velocity by the release of compressed air, resulting in low pressure at the exit region due to the Bernoulli effect. See, e.g., U.S. Pat. No. 5,511,726. The low pressure is used to draw the fluid to be aerosolized out of a second tube. This fluid breaks into small droplets as it accelerates in the air stream. Types of nebulizers for liquid formulation aerosolization include, for example, air jet nebulizers, liquid jet nebulizers, ultrasonic nebulizers, and vibrating mesh nebulizers. Nonlimiting examples of nebulizers include the Akita™ (Activaero GmbH) (see U.S. Pat. No. 7,766,012, EP1258264 and the portable Aeroneb™ Go, Pro, and Lab nebulizers (AeroGen). The nebulizer can use a pharmaceutical composition that includes any pharmaceutically acceptable carrier, including a saline solution. A dry powder formulation can also be delivered by a nebulizer. Nebulizers can be customized for delivery of the particular protein, e.g. a neutralizing anti-S protein antibody, to reduce any denaturation, aggregation, and loss of activity during nebulization.

Ultrasonic nebulizers use flat or concave piezoelectric disks submerged below a liquid reservoir to resonate the surface of the liquid reservoir, forming a liquid cone which sheds aerosol particles from its surface (U.S. 2006/0249144 and U.S. Pat. No. 5,551,416). Since no airflow is required in the aerosolization process, high aerosol concentrations can be achieved. Smaller and more uniform liquid respirable dry particles can be obtained by passing the liquid to be aerosolized through micron-sized holes. See, e.g., U.S. Pat. Nos. 6,131,570; 5,724,957; and 6,098,620.

Vibrating mesh nebulizers, which are considered less likely to cause protein denaturation (See, Bodier-Montagutelli et al. (2018) Exp Op Drug Deliv 15:729-736), may be used to generate aerosols for delivery of a liquid composition the includes a neutralizing anti-S1 antibody to the lungs of a subject. Vibrating mesh nebulizers force liquid through a vibrating membrane with apertures of specific sizes, resulting in droplets having diameters within a specified range, and may be customized for optimal delivery and stability of specific protein formulations. Nonlimiting examples of vibrating mesh nebulizers include the ALX-0171 Nanobody™ nebulizer, the Vectura FOS-Flamingo®, the PARI eFlow®, the Philips I-neb AAD®, and the Aeroneb™ Pro (Rohm et al. (2017) Intl J Pharmaceutics 532:537-546; Bodier-Montagutelli et al. (2018)).

In various embodiments, a dosage regimen can include a single dose of a liquid formulation of the invention, of 0.001 to 500 mg neutralizing anti-S protein antibody, or about 10 μg to 200 mg neutralizing anti-S protein antibody, administered daily, every other day, or weekly, or a plurality of doses administered at least twice, 2-3 times, 2-4 times or 2-6 times daily; or a plurality of doses administered once every 36 hours, once every 36-48 hours, once every 36-72 hours, once every 2-3 days, once every 2-4 days, once every 2-5 days, or once every week; or a plurality of doses administered once every 36 hours, once every 36-48 hours, once every 36-72 hours, once every 2-3 days, once every 2-4 days, once every 2-5 days, or once every week.

In some embodiments, the method further comprises detecting reduced infection or reduced viral load of the coronavirus in a subject diagnosed as having a coronavirus infection after pulmonary delivery of a neutralizing antibody as provided herein to the subject.

The disclosure provides methods for treating coronavirus infection by delivering to the lungs of a subject having or suspected of having a coronavirus infection a composition that includes a polypeptide comprising a neutralizing anti-S protein antibody formulated for pulmonary administration. The composition can be a pharmaceutical composition that includes, in addition to a neutralizing anti-S protein antibody, at least one pharmaceutically acceptable excipient or carrier compound. The pharmaceutical formulation that includes a neutralizing anti-51 antibody can be a formulation for delivery by aerosol inhalation such as by a nebulizer and can be in liquid form. Compositions as provided herein can be packaged in single dose units, for example, in vials or dispensers such as nebulizers that generate aerosols for delivery of particles

In addition to administration to the lungs by or droplets to the lung.inhalation, for example, through the use of a nebulizer, dry powder inhaler, or metered dose inhaler, or a liquid pharmaceutical composition that includes a neutralizing antibody as provided herein can be formulated for intranasal topical nasal application, for example, for delivering up to approximately 1-3 ml of nasal formation in the nostrils. As used herein, topical nasal delivery does not use an inhalation device such as a nebulizer, dry powder inhaler, or metered dose inhaler. Nasal formulations for topical delivery are described hereinabove, and can be, for example, liquid, gel-based, or in the form of a paste or dry powder for administration to the nasal passages. Provided herein are methods of treating or preventing a coronavirus infections where the methods include administering a composition that includes a neutralizing antigen-binding protein as provided herein that binds the S protein of a coronavirus to the nasal passages of a subject infected with coronavirus, suspected of being infected with a coronavirus, or at risk of becoming infected with a coronavirus, such as, but not limited to, SARS-CoV-2 or a variant thereof. The method includes applying a liquid, gel, paste, or powder composition that includes a neutralizing antigen binding protein as disclosed herein that binds the S protein of a coronavirus to the nasal passages of a subject. The composition can include, for example, an antigen binding protein that binds the S protein of SARS-CoV-2 that includes a heavy chain variable domain sequence having at least 95%, at least 96%, at least 97%, at least 98%, or at least 99% sequence identity to the amino acid sequence of SEQ ID NO:28 and a light chain variable domain sequence having at least 95%, at least 96%, at least 97%, at least 98%, or at least 99% sequence identity to the amino acid sequence of SEQ ID NO:29, where the antigen-binding protein specifically binds the S protein of SARS-CoV-2 and has neutralizing activity with respect to at least one coronavirus, e.g., SARS-CoV-2. In various embodiments a neutralizing antibody as disclosed herein specifically binds the S protein of SARS-CoV-2 and includes heavy chain CDR1, CDR2, and CDR3 sequences of SEQ ID NO:30, SEQ ID NO:31, and SEQ ID NO:32, respectively, and further includes light chain CDR1, CDR2, and CDR3 sequences of SEQ ID NO:33, SEQ ID NO:34, and SEQ ID NO:35, respectively. In various embodiments the antigen binding protein is an antibody, such as a fully human IgG antibody, having the heavy chain variable domain sequence of SEQ ID NO:28 and the light chain variable domain sequence of SEQ ID NO:29. The antibody can further include an Fc region having at least one mutation, such as the LALA mutation. In some embodiments the antibody in an intranasal pharmaceutical composition used in the methods is STI-2020.

The pharmaceutical composition can be deposited as a powder, gel, paste, or liquid, such as by liquid drops that can be administered by the subject or by a health professional. In some embodiments, during application of the pharmaceutical composition, the subject is in a horizontal (supine) position or has the head tilted back such that the nasal passages are in a horizontal or near-horizontal orientation for a period of time of from a few seconds to about 10 minutes or more to allow for retention or adhesion of the composition in the nasal passages during and immediately following delivery of the composition. The concentration of neutralizing antibody in the pharmaceutical composition can be, as nonlimiting examples, between about 1 μg/mL and about 1 g/mL, between about 50 μg/mL and about 500 mg/mL, between about 100 μg/mL and about 250 mg/mL, between about 1 mg/mL and about 200 mg/mL, or between about 2 mg/mL and about 100 mg/mL, such as between about 5 mg/mL and about 100 mg/mL, or from between about 10 mg/mL and about 50 mg/mL. These values are exemplary and not limiting. The composition can be deposited in the nasal passages of a subject in a volume of from about 50 μl to about 4 ml for a single application, such as for example about 100 μl to about 3 ml or about 50 μl to about 2 ml or from about 100 μl to about 1 ml for a single nostril application. The dosing can make use of a dropper, syringe, squeeze bottle, or the like, or for a gel, paste, or powder can use an applicator. The application of a single dosing can be to one or both nostrils. The dosing of a subject can be a single dosing or can be multiple dosings, for example over a period of days. For example, a subject can be dosed daily, every other day, twice per week, weekly, or biweekly, where the dosing can be optionally performed by the subject outside of a clinical setting (for example, in the home).

In further embodiments a subject may be treated with an intranasal pharmaceutical composition as provided herein, such as an intranasal composition formulated for topical delivery to the nasal passages of the subject, and may also be treated with an intravenous pharmaceutical composition as provided herein. For example, a subject may be treated with an intranasal composition and an intravenous composition on the same day or on different days, and may receive more than one dose of either or both of the intranasal composition and the intravenous composition.

### Methods of Detecting Coronavirus

The present disclosure provides methods (e.g., in vitro) for detecting the presence of a coronavirus, or a protein from a coronavirus, in a sample, comprising: (a) contacting the sample (containing a target antigen) with any one or any combination of two or more of the neutralizing antibodies having increased in vivo serum half-life and/or reduced effector function described herein, under conditions suitable to form an antibody-antigen complex; and (b) detecting the presence of the antibody-antigen complex. In one embodiment, this method can be used to detect the presence of a coronavirus in a sample from a subject and thus can be used to diagnose a subject suspected of having a coronavirus infection. In one embodiment, the sample from the subject comprises phlegm, saliva, blood, cheek scaping, tissue biopsy, hair or semen. In one embodiment, the sample from the subject can be obtained from an acutely coronavirus infected subject or a convalescing subject. In one embodiment, the subject can be human, non-human primates, simian, ape, murine (e.g., mice and rats), bovine, porcine, equine, canine, feline, caprine, lupine, ranine or piscine. In one embodiment, the sample can comprise cells expressing a coronavirus membrane protein, or a coronavirus. In one embodiment, the neutralizing antibodies can be labeled so permit detection of an antigen-antibody complex, where the label comprises a radionuclide, fluorescer, enzyme, enzyme substrate, enzyme cofactors, enzyme inhibitors and ligands (e.g., biotin, haptens). In one embodiment, the presence of the antibody-antigen complex can be detected using any detection mode including radioactive, colorimetric, antigenic, enzymatic, a detectable bead (such as a magnetic or electrodense (e.g., gold) bead), biotin, streptavidin or protein A.

The present disclosure further provides methods (e.g., in vitro) for identifying a compound that modulates (increases or decreases) binding between a coronavirus S protein and an ACE2 target receptor (or cells expressing ACE2 e.g., Vero E6 cells), comprising: (a) contacting (i) a candidate compound with (ii) a coronavirus S protein and with (iii) any one or any combination of two or more of the neutralizing antibodies having increased in vivo serum half-life and/or reduced effector function described herein, under conditions suitable to form an antibody-S1 complex; and (b) detecting the presence or absence of the antibody-S protein complex. In one embodiment, the lack of formation of the complex may indicate that the candidate compound competes for the same or overlapping epitope on the S protein subunit as the neutralizing antibody. In one embodiment, the concentration of the candidate compound and/or the neutralizing antibody can be increased to obtain a dose response curve. In one embodiment, the neutralizing antibodies can be labeled so permit detection of an antigen-antibody complex, where the label comprises a radionuclide, fluorescer, enzyme, enzyme substrate, enzyme cofactors, enzyme inhibitors and ligands (e.g., biotin, haptens). In one embodiment, the presence of the antibody-antigen complex can be detected using any detection mode including radiation, fluorescence, colorimetric, absorption wavelength, electron densityantigenic, enzymatic, a detectable bead (such as a magnetic or electrodense (e.g., gold) bead), biotin, streptavidin or protein A.

SEQUENCES

SEQ ID NO: 1
Protein
SARS-Cov-2 spike (S) protein (including leader sequence) with S1 and S2 subunits
Genbank ACCESSION QHD43416
MFVFLVLLPLVSSQCVNLTTRTQLPPAYTNSFTRGVYYPDKVERSSVLHSTQDLFLPFFS
NVTWFHAIHVSGTNGTKRFDNPVLPENDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGESALEPLVDLPIGINITREQT
LLALHRSYLTPGDSSSGWTAGAAAYYVGYLQPRTELLKYNENGTITDAVDCALDPLSETK
CTLKSFTVEKGIYQTSNERVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLERKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKELPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGDC
LGDIAARDLICAQKENGLTVLPPLLTDEMIAQYTSALLAGTITSGWTFGAGAALQIPFAM
QMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNETTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKEDEDD
SEPVLKGVKLHYT
SEQ ID NO: 2
Protein
SARS-Cov-2 spike protein without leader sequence, that includes the S1 subunit and
the S2 subunit up to amino acid 1213 (amino acids 16-1213 of SEQ ID NO: 1)
VNLTTRTQLPPAYTNSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGVYYHKNNKSWMES
EFRVYSSANNCTFEYVSQPFLMDLEGKQGNEKNLREFVEKNIDGYFKIYSKHTPINLVRDLPQGESALEP
LVDLPIGINITRFQTLLALHRSYLTPGDSSSGWTAGAAAYYVGYLQPRTELLKYNENGTITDAVDCALDP
LSETKCTLKSFTVEKGIYQTSNERVQPTESIVRFPNITNLCPFGEVENATRFASVYAWNRKRISNCVADY
SVLYNSASFSTFKCYGVSPTKLNDLCFTNVYADSFVIRGDEVRQIAPGQTGKIADYNYKLPDDETGCVIA
WNSNNLDSKVGGNYNYLYRLFRKSNLKPFERDISTEIYQAGSTPCNGVEGENCYFPLQSYGFQPTNGVGY
QPYRVVVLSFELLHAPATVCGPKKSTNLVKNKCVNFNFNGLTGTGVLTESNKKELPFQQFGRDIADTTDA
VRDPQTLEILDITPCSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARSVASQSIIAYTMSLGAENSVAYSNNSIAIP
TNFTISVTTEILPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLENKVTLADAGFIKQYGDCLGDIAARDLICAQKF
NGLTVLPPLLTDEMIAQYTSALLAGTITSGWTFGAGAALQIPFAMQMAYRENGIGVTQNVLYENQKLIAN
QFNSAIGKIQDSLSSTASALGKLQDVVNQNAQALNTLVKQLSSNFGAISSVLNDILSRLDKVEAEVQIDR
LITGRLQSLQTYVTQQLIRAAEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDRLNEVAKNLNESLIDLQELGK
YEQYIKWP
SEQ ID NO: 3
SARS-Cov-2 spike S1 subunit (including leader sequence)
MFVFLVLLPLVSSQCVNLTTRTQLPPAYTNSFTRGVYYPDKVFRSSVLHSTQDLFLPFFS
NVTWFHAIHVSGTNGTKRFDNPVLPENDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTELLKYNENGTITDAVDCALDPLSETK
CTLKSFTVEKGIYQTSNERVQPTESIVREPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKELPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRAR
SEQ ID NO: 4
SARS-Cov-2 spike S1 subunit (no leader sequence)
Amino acids 16-685 of SEQ ID NO: 1
VNLTTRTQLPPAYTNSFTRGVYYPDKVFRSSVLHSTQDLFLPFFSNVTWFHAIHVSGTNGTKREDNPVLP
FNDGVYFASTEKSNIIRGWIFGTTLDSKTQSLLIVNNATNVVIKVCEFQFCNDPFLGVYYHKNNKSWMES
EFRVYSSANNCTFEYVSQPFLMDLEGKQGNFKNLREFVEKNIDGYFKIYSKHTPINLVRDLPQGESALEP
LVDLPIGINITRFQTLLALHRSYLTPGDSSSGWTAGAAAYYVGYLQPRTELLKYNENGTITDAVDCALDP
LSETKCTLKSFTVEKGIYQTSNERVQPTESIVREPNITNLCPFGEVENATRFASVYAWNRKRISNCVADY
SVLYNSASFSTFKCYGVSPTKLNDLCFTNVYADSFVIRGDEVRQIAPGQTGKIADYNYKLPDDETGCVIA
WNSNNLDSKVGGNYNYLYRLERKSNLKPFERDISTEIYQAGSTPCNGVEGENCYFPLQSYGFQPTNGVGY
QPYRVVVLSFELLHAPATVCGPKKSTNLVKNKCVNFNFNGLTGTGVLTESNKKELPFQQFGRDIADTTDA
VRDPQTLEILDITPCSFGGVSVITPGTNTSNQVAVLYQDVNCTEVPVAIHADQLTPTWRVYSTGSNVFQT
RAGCLIGAEHVNNSYECDIPIGAGICASYQTQTNSPRRAR
SEQ ID NO: 5
SARS-Cov-2 spike protein RBD
amino acids 319-537 of SEQ ID NO: 1
RVQPTESIVRFPNITNLCPFGEVENATRFASVYAWNRKRISNCVADYSVLYNSASFSTFKCYGVSPTKLN
DLCFTNVYADSFVIRGDEVRQIAPGQTGKIADYNYKLPDDFTGCVIAWNSNNLDSKVGGNYNYLYRLERK
SNLKPFERDISTEIYQAGSTPCNGVEGENCYFPLQSYGFQPTNGVGYQPYRVVVLSFELLHAPATVCGPK
KSTNLVKNK
SEQ ID NO: 6
mAb S1D2 VH
EVQLVESGGGLIQPGGSLRLSCAASGFTVSSNYMSWVRQAPGKGLEWVSIIYPGGSTNYADSVKGRFTIS
RDNSRNTLYLQMNSLRAEDTAVYYCARELGYYGMDVWGQGTTVTVSS
SEQ ID NO: 7
mAb S1D2 VL
DIQMTQSPSSVSASVGDRVTITCRASQGISTWLVWYQQKPGKAPNLLIYGASSLQSGVPSRESGSGSGTD
FTLTISSLQPEDFATYYCQQANSFPYTFGQGTKLEIK
SEQ ID NO: 8
mA S1D2 Heavy chain CDR1
SNYMS
SEQ ID NO: 9
mA S1D2 Heavy chain CDR2
IIYPGGSTNYADSVKG
SEQ ID NO: 10
mA S1D2 Heavy chain CDR3
ELGYYGMDV
SEQ ID NO: 11
mA S1D2 Light chain CDR1
RASQGISTWLV
SEQ ID NO: 12
mA S1D2 Light chain CDR2
GASSLQS
SEQ ID NO: 13
mA S1D2 Light chain CDR3
QQANSFPYT
SEQ ID NO: 14
Human Fc region with LALA mutations (L234A, L235A)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPEAAGGPSVFLFPPKPKDTLMISRTP
EVTCVVVDVSHEDPEVKENWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLY SKLTVDKSRWQQGNVFSCSVMHEALHNHYTQKSLSLSPGK
SEQ ID NO: 15
Human Fc region with triple mutation YTE: M252Y, S254T, T256E
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGGPSVELFPPKPKDTLYITREP
EVTCVVVDVSHEDPEVKENWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 16
Human Fc region with LALA and YTE mutations:
(L234A, L235A) (M252Y, S254T, T256E)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPEAAGGPSVFLFPPKPKDTLYITREP
EVTCVVVDVSHEDPEVKENWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLY SKLTVDKSRWQQGNVFSCSVMHEALHNHYTQKSLSLSPGK
SEQ ID NO: 17
mouse Ig gamma leader peptide sequence
MEWSWVFLFFLSVTTGVHS
SEQ ID NO: 18
lower hinge/CH2 sequence
PAPELLGGP
SEQ ID NO: 19
Fc region sequence (CH2)
SVFLFPPKPKDT
SEQ ID NO: 20
hinge region
EPKSCDKTHTCPPCPAPELLGGP
SEQ ID NO: 21
linker
GGGGSGGGGSGGGGS
SEQ ID NO: 22
Protein
Homo sapiens
angiotensin-converting enzyme 2 (ACE2) precursor
NCBI Reference Sequence: NP_068576.1 UniProtKB Q9BYF1
Signal peptide is underlined
805 amino acids
MSSSSWLLLSLVAVTAAQSTIEEQAKTFLDKFNHEAEDLFYQSSLASWNYNTNITEENVQNMNNAGDKWS
AFLKEQSTLAQMYPLQEIQNLTVKLQLQALQQNGSSVLSEDKSKRLNTILNTMSTIYSTGKVCNPDNPQE
CLLLEPGLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGREWTNLYS
LTVPFGQKPNIDVTDAMVDQAWDAQRIFKEAEKFFVSVGLPNMTQGFWENSMLTDPGNVQKAVCHPTAWD
LGKGDFRILMCTKVTMDDELTAHHEMGHIQYDMAYAAQPELLRNGANEGFHEAVGEIMSLSAATPKHLKS
IGLLSPDFQEDNETEINFLLKQALTIVGTLPFTYMLEKWRWMVFKGEIPKDQWMKKWWEMKREIVGVVEP
VPHDETYCDPASLFHVSNDYSFIRYYTRTLYQFQFQEALCQAAKHEGPLHKCDISNSTEAGQKLENMLRL
GKSEPWTLALENVVGAKNMNVRPLLNYFEPLFTWLKDQNKNSFVGWSTDWSPYADQSIKVRISLKSALGD
KAYEWNDNEMYLFRSSVAYAMRQYFLKVKNQMILFGEEDVRVANLKPRISENFFVTAPKNVSDIIPRTEV
EKAIRMSRSRINDAFRLNDNSLEFLGIQPTLGPPNQPPVSIWLIVFGVVMGVIVVGIVILIFTGIRDRKK
KNKARSGENPYASIDISKGENNPGFQNTDDVQTSF
SEQ ID NO: 23
Homo sapiens
protein
ACE2 receptor polypeptide soluble ectodomain, no signal peptide (amino acids 18-740)
QSTIEEQAKTFLDKENHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGREWTNLYSLTVPFGQKPNIDVTDAM
VDQAWDAQRIFKEAEKFFVSVGLPNMTQGFWENSMLTDPGNVQKAVCHPTAWDLGKGDERILMCTKVTMD
DELTAHHEMGHIQYDMAYAAQPFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLENMLRLGKSEPWTLALENVVGAK
NMNVRPLLNYFEPLFTWLKDQNKNSFVGWSTDWSPYADQSIKVRISLKSALGDKAYEWNDNEMYLERSSV
AYAMRQYFLKVKNQMILFGEEDVRVANLKPRISENFFVTAPKNVSDIIPRTEVEKAIRMSRSRINDAFRL
NDNSLEFLG
SEQ ID NO: 24
Homo sapiens
protein
ACE2 receptor polypeptide soluble ectodomain, no signal peptide (amino acids 18-615
QSTIEEQAKTFLDKENHEAEDLFYQSSLASWNYNTNITEENVQNMNNAGDKWSAFLKEQSTLAQMYPLQE
IQNLTVKLQLQALQQNGSSVLSEDKSKRLNTILNTMSTIYSTGKVCNPDNPQECLLLEPGLNEIMANSLD
YNERLWAWESWRSEVGKQLRPLYEEYVVLKNEMARANHYEDYGDYWRGDYEVNGVDGYDYSRGQLIEDVE
HTFEEIKPLYEHLHAYVRAKLMNAYPSYISPIGCLPAHLLGDMWGREWTNLYSLTVPFGQKPNIDVTDAM
VDQAWDAQRIFKEAEKFFVSVGLPNMTQGFWENSMLTDPGNVQKAVCHPTAWDLGKGDERILMCTKVTMD
DELTAHHEMGHIQYDMAYAAQPELLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLENMLRLGKSEPWTLALENVVGAK
NMNVRPLLNYFEPLFTWLKDQNKNSFVGWSTDWSPYAD
SEQ ID NO: 25
Protein
human IgG1 Fc region (amino acids 100-330 of Genbank P01857) (“Fc tag”)
PKSCDKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLSPGK
SEQ ID NO: 26
DNA
Cytomegalovirus
CMV enhancer plus promoter
CGTTACATAACTTACGGTAAATGGCCCGCCTGGCTGACCGCCCAACGACCCCCGCCCATTGACGTCAATA
ATGACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGACGG
TAAATGGCCCGCCTGGCATTATGCCCAGTACATGACCTTATGGGACTTTCCTACTTGGCAGTACATCTAC
GTATTAGTCATCGCTATTACCATGGTGATGCGGTTTTGGCAGTACATCAATGGGCGTGGATAGCGGTTTG
ACTCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGGAG
GTCTATATAAGCAGAGCT
SEQ ID NO: 27
Protein
SARS-Cov-2 spike S1 subunit (no leader sequence), polyhistidine tag
VNLTTRTQLPPAYTNSFTRGVYYPDKVERSSVLHSTQDLFLPFFSNVTWFHAIHVSGTNGTKREDNPVLP
FNDGVYFASTEKSNIIRGWIFGTTLDSKTQSLLIVNNATNVVIKVCEFQFCNDPFLGVYYHKNNKSWMES
EFRVYSSANNCTFEYVSQPFLMDLEGKQGNEKNLREFVEKNIDGYFKIYSKHTPINLVRDLPQGESALEP
LVDLPIGINITRFQTLLALHRSYLTPGDSSSGWTAGAAAYYVGYLQPRTELLKYNENGTITDAVDCALDP
LSETKCTLKSFTVEKGIYQTSNERVQPTESIVREPNITNLCPFGEVENATRFASVYAWNRKRISNCVADY
SVLYNSASFSTFKCYGVSPTKLNDLCFTNVYADSFVIRGDEVRQIAPGQTGKIADYNYKLPDDFTGCVIA
WNSNNLDSKVGGNYNYLYRLERKSNLKPFERDISTEIYQAGSTPCNGVEGENCYFPLQSYGFQPTNGVGY
QPYRVVVLSFELLHAPATVCGPKKSTNLVKNKCVNENENGLTGTGVLTESNKKELPFQQFGRDIADTTDA
VRDPQTLEILDITPCSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARHHHHHH
SEQ ID NO: 28
Protein
S1D7270 antibody, Heavy Chain Variable region
EVQLVESGGGLIQPGGSLRLSCAASGFTVSSNYMSWVRQAPGKGLEWVSIIYPGGSTEYADSVKGRFTIS
RDNSRNTLYLQMNSLRAEDTAVYYCARELGYYGMDVWGQGTTVTVSS
SEQ ID NO: 29
Protein
SID7270 antibody, Light Chain Variable region
DIQMTQSPSSVSASVGDRVTITCRASQGISTWLVWYQQKPGKAPNLLIYGASSLQSGVPSRFSGSGSGTD
FTLTISSLQPEDFATYYCQQANAYPYTFGQGTKLEIK
SEQ ID NO: 30
Protein
S1D7270 antibody, Heavy Chain CDR1
SNYMS
SEQ ID NO: 31
Protein
SID7270 antibody, Heavy Chain CDR2
IIYPGGSTEYADSVKG
SEQ ID NO: 32
Protein
SID7270 antibody, Heavy Chain CDR3
ELGYYGMDV
SEQ ID NO: 33
Protein
S1D7270 antibody, Light Chain CDR1
RASQGISTWLV
SEQ ID NO: 34
Protein
S1D7270 antibody, Light Chain CDR2
GASSLQS
SEQ ID NO: 35
Protein
S1D7270 antibody, Light Chain CDR3
QQANAYPYT

### EXAMPLES

The following examples are meant to be illustrative and can be used to further understand embodiments of the present disclosure. The examples should not be construed as limiting the scope of the present teachings in any way.

### Example 1. S1D2 Variant Antibodies that Block Binding to ACE2

The antibody S1D2 (PCT/US2021/030909), having the variable heavy chain sequence of SEQ ID NO:6 and the variable light chain sequence of SEQ ID NO:7, was used as the “parent antibody” to engineer variant antibodies. Antibodies were screened for their ability to block binding of the S1 subunit of SARS-CoV-2 to the ACE2 ectodomain. The ACE2 ectodomain polypeptide used in the assay was a polypeptide that included the amino acid sequence of SEQ ID NO:23 fused to an Fc region sequence (SEQ ID NO:25).

A 96-well ELISA plate (CORNING 3690) was coated with 3 μg/mL ACE2-Fc overnight at 4° C. The plate was then washed with PBS-T three times. Antibodies were serially diluted two-fold starting at a concentration of 100 μg/mL. For each dilution, the antibody was mixed 1:1 with 1.25 μg/mL SARS-CoV-2 S1 subunit protein (SEQ ID NO:4) with a carboxy terminal his tag (Acrobiosystems catalog #S1N-C52H3). The antibody-S1 subunit protein mixtures (25 l) were transferred to the wells of the ELISA plate and incubated 30 min with shaking. The plate was then washed three times with PBS-T. Rabbit anti-His polyclonal antibody-HRP diluted in Blocker Casein in PBS (1:5000) was added to each well and then the TMB substrate was added. The reaction was allowed to develop for 30 min. 2M H₂SO₄ was used to stop the reaction and the OD was read at 450 nm.

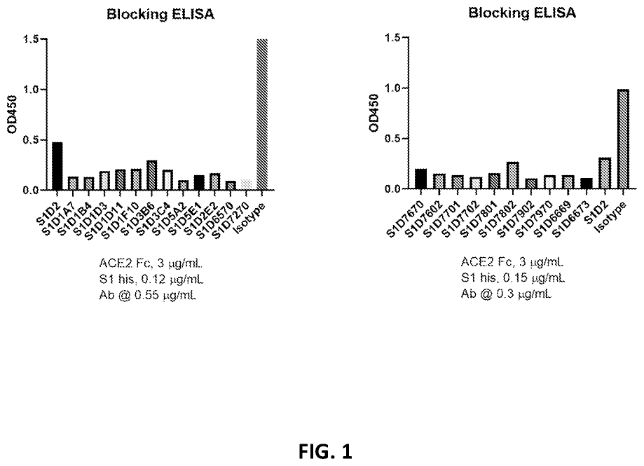

FIG. 1 provides the results of the blocking assay. All 23 variant antibodies demonstrated stronger inhibition of the interaction between the SARS-CoV-2 S1 subunit and ACE2 than parental antibody S1D2.

### Example 2. S1D2 and S1D7270 Antibody Binding Affinities

Binding kinetics of anti-spike protein antibodies with the RBD of the SARS-CoV-2 S protein was measured using surface plasmon resonance (SPR). Antibodies (parental antibody S1D2 and variants of S1D2) were immobilized on a BIACORE CM5 sensor chip to approximately 500 RU using standard N-hydroxysuccinimide/N-Ethyl-N′-(3-dimethylaminopropyl) carbodiimide hydrochloride (NHS/EDC) coupling methodology.

Recombinant SARS-CoV2 RBD (SEQ ID NO:5) having a carboxy terminal his tag was serially diluted in a running buffer of 0.01 M HEPES pH 7.4, 0.15 M NaCl, 3 mM EDTA, 0.05% v/v Surfactant P20 (HBS-EP+). All measurements were conducted in HBS-EP+ buffer with a flow rate of 30 μL/minute. The affinity of S1D2 was analyzed with BIAcore T200 Evaluation software 3.1. A 1:1 (Langmuir) binding model was used to fit the data. All BIACORE assays were performed at room temperature.

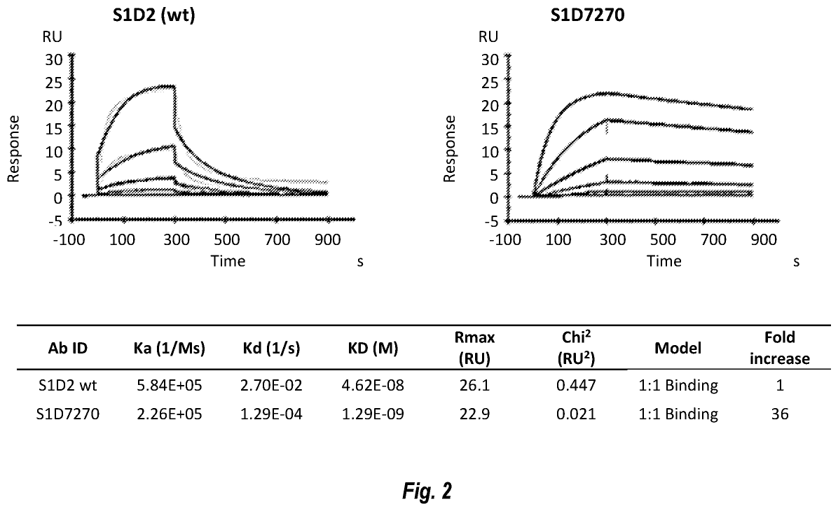

The SPR sensorgrams of parental anti-S1 antibody S1D2 and variant antibody S1D7270 binding to the SARS-CoV2 spike protein RBD are shown in FIG. 2. The S1D2 antibody was found to have a binding affinity (K_{d}) of 46 nM for the SARS-CoV2 S protein RBD, and variant S1D7270 was found to have a binding affinity (K_{d}) of 1.29 nM for the SARS-CoV2 S protein RBD.

### Example 3. ACE2-S1 Interaction Inhibition ELISA

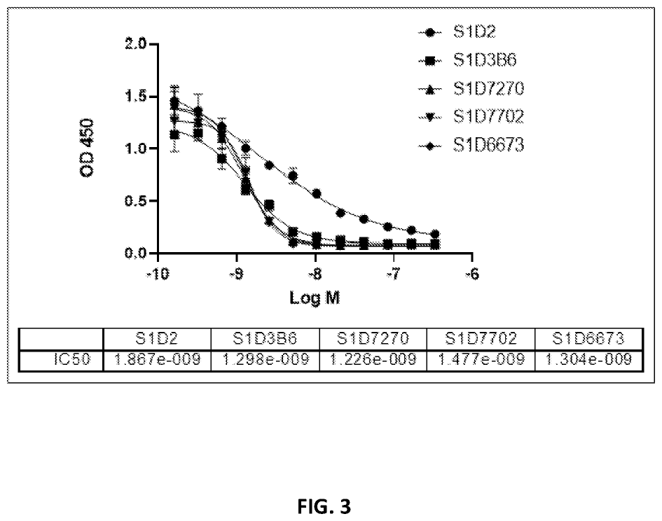

The wells of a 96 well plate were coated with 3 μg/mL recombinant ACE2-Fc (the ACE2 polypeptide comprising the amino acid sequence of SEQ ID NO:23 fused to an Fc region (SEQ ID NO:25)) overnight at 4° C. The plates were washed three times with PBS-Tween PBS-T and blocked for 1 hour with 170 μL Blocker Casein in PBS at room temperature. The plates were then washed three times with PBS-T. Two-fold serial dilutions of antibody (parental antibody S1D2 and variants S1D3B6, S1D7270, S1D7702, and S1D6673) starting from a concentration of 60 μg/mL were mixed 1:1 with 3 μg/mL recombinant SARS-CoV-2 S1 protein (SEQ ID NO:4) that included a his tag. Twenty-five μL of the antibody dilutions/S1 protein mixtures were added to the ELISA plate and incubated for 1 hour with shaking. The plate was washed three times with PBS-T, then rabbit anti-His polyclonal antibody-HRP (diluted at 1:5000 in casein) was added and the plate was incubated for 1 hour. Subsequently, TMB (3,3′,5,5-tetramethylbenzidine) was added as substrate and developed for 30 minutes. The reaction was stopped using 2 M H₂SO₄ and the OD was read at 450 nm. GraphPad Prism 8.3.0 software was used to analyze the data. The ELISA results are shown in FIG. 3. The IC₅₀ of the S1D7270 antibody for S1 binding was determined to be 1.226 nM in this assay, lower than the IC₅₀ of the parental S1D2 antibody (4.883 nM).

### Example 4. Binding of S1D2 and SID7270 to Cells Expressing SARS-CoV-2 Spike Protein and Spike Protein Variants

To assess binding of the S1D2 and S1D7270 antibodies (each of which included the LALA mutation in the Fc region) to the spike protein expressed on HEK293T cells surface by FACS, HEK293 cells were transfected using the Fugene reagent with mammalian expression vectors that included either a gene encoding the SARS-CoV-2 spike protein or a gene encoding the SARS-CoV-2 D614G mutant spike protein. Forty-eight hours post-transfection, cells were harvested using enzyme free cell dissociation buffer (ThermoFisher), washed once, and resuspended in FACS buffer (DPBS+2% FBS) at 4×10⁶ cells/ml. For antibody binding to the cells expressing the Spike proteins, the cells were dispensed into wells of a 96-well plate and an equal volume of 2× final concentration of serially diluted anti-S1 antibody solution was added. After incubation on ice for 45 minutes, the cells were washed with FACS buffer. Detection of bound antibody was carried out by staining the cells with 50 μl of 1:500 diluted APC AffiniPure F(ab′)2 Fragment (Goat Anti-Human IgG (H+L) Jackson ImmunoResearch) for 20 minutes on ice. The cells were washed once with 150 μl FACS buffer and analyzed using flow cytometry. A sigmoidal four-parameter logistic equation was used for fitting the MFI vs. mAb concentration data set to extract EC50 values (GraphPad Prism 8.3.0 software).

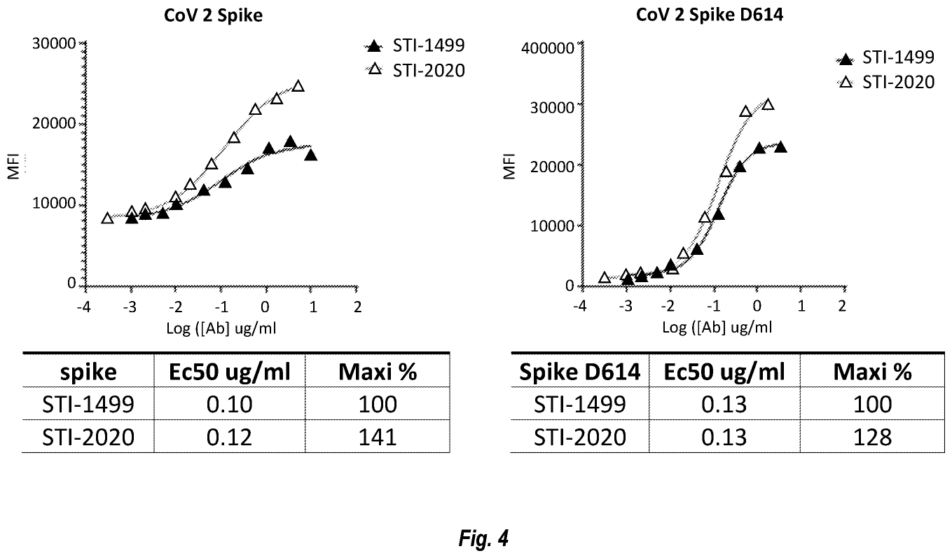

The FACS results are shown in FIG. 4. The results show that both the S1D2 and S1D7270 antibodies efficiently bound the SARS-CoV-2 S wild type protein and the SARS-CoV-2 S D614G mutant S protein expressed on the surface of the cells.

### Example 5. Inhibition of CPE by S1D7270 Antibody

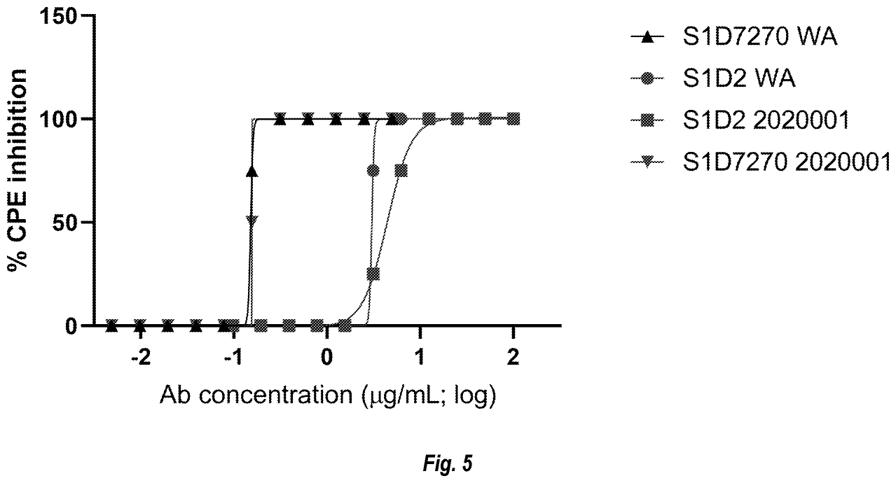

To test for inhibition of the cytopathic effect (CPE) by the S1D7270 antibody and compare it to the inhibition of CPE exhibited by the parent antibody S1D2, 2×10⁴ VeroE6 cells were plated in the wells of 96-well plates and incubated approximately 24 hours at 37° C., 5% CO₂. The S1D2_{LALA} antibody (STI-1499) and S1D7270_{LALA} antibody (STI-2020) were 2-fold serially diluted in infection media (DMEM+2% FBS) and 25 μL of the serially diluted samples were incubated with 100×50% tissue culture infective doses (TCID₅₀) of either SARS-CoV-2 strain WA or SARS-CoV-2 strain 2020001 (which expresses a spike protein having the D614G mutation) in 60 μL for 1 hour at 37° C. The antibody/virus mixtures were then used to infect monolayers of Vero E6 cells grown in the wells of 96-well plates, where each concentration sample was applied in quadruplicate wells. Virus supernatant was removed and replaced with fresh medium after 1 hour of culture at 37° C. Cytopathic effect (CPE), i.e., the appearance of plaques or discontinuity in the cell monolayer due to cell lysis, was observed daily in each well and recorded on day 3 post-infection. At the end of the study, the media was aspirated, and the cells were then fixed with formalin and stained with 0.25% crystal violet. The concentration of antibody that completely prevented CPE in 50% of the wells (NT₅₀) were calculated following the Reed & Muench method. The results are shown in FIG. 5. Both S1D2_{LALA} and S1D7270_{LALA} neutralized the SARS-CoV-2 strains. The S1D7270_{LALA} antibody neutralized both isolates with an IC50 of 0.055 μg/ml and an IC99 of 0.078 μg/ml, a greater than 50-fold enhancement over the S1D2_{LALA} antibody.

### Example 6. Treatment of SARS-CoV-2 Infection in Hamsters with STI-2020 (S1D7270_{LALA})

The effectiveness of STI-2020 was tested in hamsters as a preclinical model of Covid-19 disease, where the disease manifests as failure to gain the normal amount of weight during the growth phase. Six week old female hamsters were intranasally inoculated with 1×10⁵ TCID50 of the “wild type” SARS-CoV-2 virus (USA/WA-1/2020) in 100 μl PBS. The inoculated animals were treated one-hour post-infection with titrated doses of STI-2020 or an isotype antibody control (IsoCtl, 500 μg) administered intravenously in up to 350 μl PBS. Animals in STI-2020 treatment groups of 10 animals each were administered a single dose of either 100, 300, or 500 μg of the STI-2020 antibody, and uninfected animals (UI) were administered 500 μg of the isotype control IgG1 (IsoCtl) to test for any effects of antibody on growth.

Hamsters were inoculated with SARS-CoV-2 WA-1 isolate on day 0. One-hour post-infection, animals (n=10 per group) were administered a single intravenous dose of Control IgG (500 μg) or STI-2020 (30 μg, 100 μg, 300 μg, or 500 μg). Daily weight changes from day 0 to day 10 were monitored. Lung tissues collected from five animals per group on day 5 (animals were designated for lung dissection prior to the outset of treatment) and virus titers of the extracted lung tissue were determined.

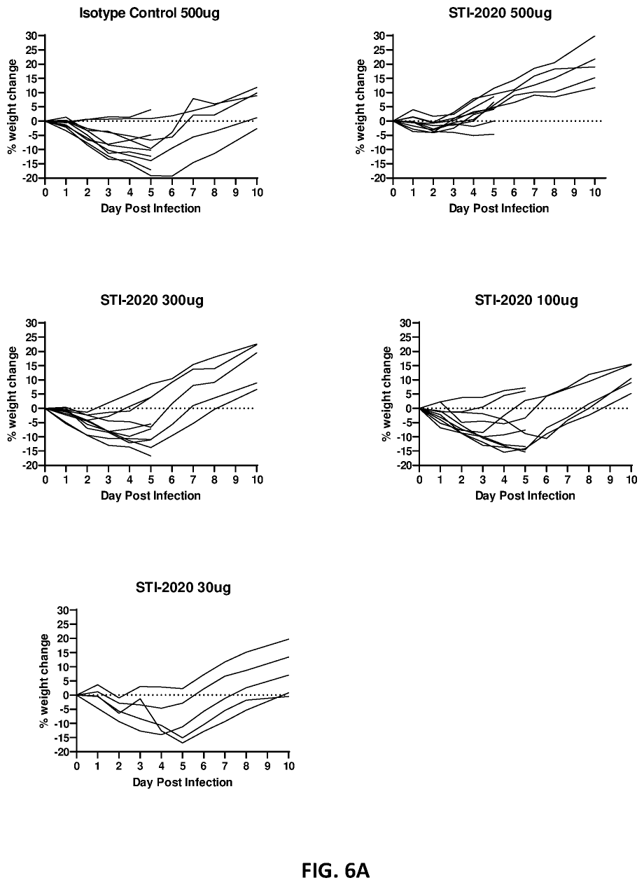

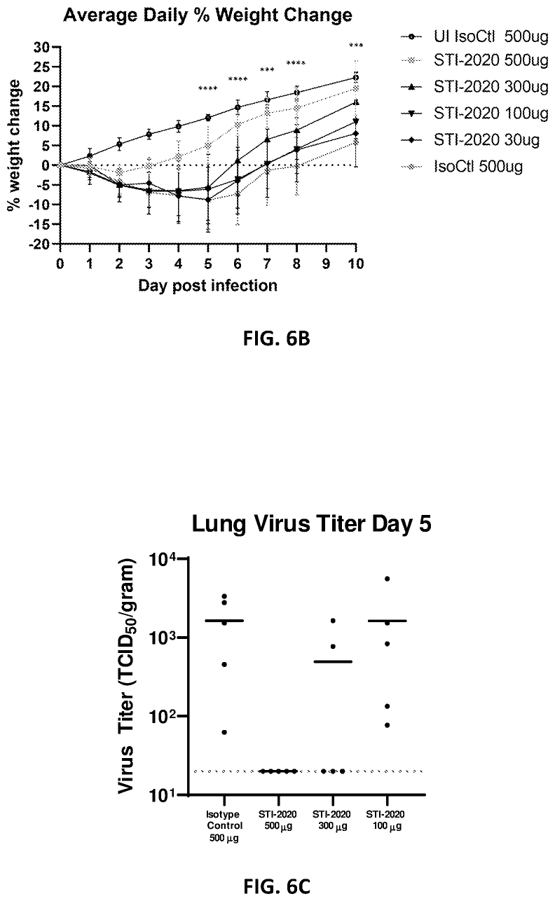

The weights of the animals were recorded daily for 10 days post-inoculation and graphed as a percentage of starting weight (FIG. 6A). Average percent daily weight change with standard deviation for each group is shown in FIG. 6B. Days on which there was a significant difference in average % weight change between STI-2020 500 μg-treated animals and Control IgG 500 μg-treated animals are denoted by ***(p-values ≤0.0003) or ****(p-values ≤0.0001). The titer of virus extracted from the lung tissue of pre-selected animals from each treatment group sacrificed on Day 5 post-inoculation is compared in the graph of FIG. 6C. In Isotype control antibody (IsoCtl)-treated animals, an average of 1.6×10³ TCID50/g of lung tissue was detected by virus CPE assay. Treatment with 500 μg STI-2020 resulted in reduction of lung titers below the level of detection in all animals tested, a STI-2020-treatment-related lung titer reduction of at least 80-fold. In the 300 μg STI-2020 treatment group, the average lung titer was diminished below the level of detection in 3 of 5 animals tested, while 2 of 5 animals had lung titers of similar magnitude to those measured in IsoCtl-treated animals. Animals with undetectable lung virus titers in the 300 μg treatment group also experienced only moderate weight loss compared to animals with detectable lung virus titers in this group (d5 values of −11.1% and −16.7%). No changes in average lung titer compared to IsoCtl-treatment were detected in animals from the 100 μg STI-2020 treatment group.

### Example 7. Biodistribution Study of STI-2020 Delivered Intravenously and Intranasally

To investigate the biodistribution of the STI-2020 neutralizing antibody delivered intravenously and intranasally, 6-8 week-old female CD-1-IGS (strain code #022) mice were obtained from Charles River Laboratories. For intravenous injection of STI-2020, 100 μL of antibody diluted in 1×HBSS was administered retro-orbitally to anesthetized animals. For intranasal (IN) treatment, antibody was diluted in 1×HBSS and administered by inhalation into the nose of anesthetized animal in a total volume of 20 μL using a pipette tip. For intravenous treatments, three different dosages of 0/005 mg/kg, 0.05 mg/kg, and 0.5 mg/kg and a control formulation with no antibody were delivered. For intranasal treatments, four different dosages of 0/005 mg/kg, 0.05 mg/kg, 0.5 mg/kg, and 2.5 mg/kg and a control formulation with no antibody were delivered. Each treatment group consisted of five animals.

Twenty-four hours following administration of a single antibody dose, samples of serum, lung lavage, and tissues including spleen, lung, small intestine, and large intestine were obtained from each of 5 treated mice at each dose level. Organs, blood, and lung lavage samples were collected 24 hours post-antibody administration. Blood was collected by retro-orbital bleeding and then transferred to Microvette 200 Z-Gel tubes (Cat no #20.1291, lot #8071211, SARSTEDT). The tubes were then centrifuged at 10,000×g for 5 minutes at room temperature. Serum was transferred into 1.5 ml tubes and stored at −80° C. Lung lavage samples were collected following insertion of a 20G×1-inch catheter (Angiocath Autoguard, Ref #381702, lot #6063946, Becton Dickinson) into the trachea. A volume of 0.8 ml of PBS was drawn into a syringe, placed into the open end of the catheter, and slowly injected and aspirated 4 times. The syringe was removed from the catheter, and the recovered lavage fluid was transferred into 1.5 ml tubes and kept on ice. Lavage samples were centrifuged at 800×g for 10 min at 4° C. Supernatants were collected, transferred to fresh 1.5 mL tubes, and stored at −80° C. To determine the amount of antibody in organs, total spleen, 200 mg of lung, total large intestine, and 200 mg of small intestine were suspended in 300 μL of PBS in pre-filled 2.0 ml tubes containing zirconium beads (cat no #155-40945, Spectrum). Tubes were processed in a BeadBug-6 homogenizer at a speed setting of 3000 and a 30 second cycle time for four cycles with a 30-second break after each cycle. Tissue homogenates were centrifuged at 15,000 rpm for 15 minutes at 4° C. Homogenate supernatants were then transferred into 1.5 ml tubes and stored at −80° C.

STI-2020 antibody levels in each sample were quantified using the antibody detection ELISA method. Multi-Array 96-well plates (cat #L15λA-3, Meso Scale Discovery (MSD)) were coated with mouse anti-human IgG antibody (CH2 domain, cat #MA5-16929, ThermoFisher Scientific) at 2 μg/mL in 1×PBS (50 μL/well), sealed, and incubated overnight at 4° C. The following day, plates were washed 3× with 1× washing solution (KPL wash solution, cat no #5150-0009, lot no #10388555, Sera Care). Plates were then blocked using 50 μL/well of Blocker™ Casein in PBS (cat no #37528, lot #QE220946, ThermoFisher) for 1 hour at room temperature on an orbital shaker. Plates were washed 3× with 1× washing solution. Samples from biodistribution or pharmacokinetic experiments were added in a volume of 50 μL to each well. STI-2020 antibody was serially diluted from a concentration of 1000 ng/ml to 3.1 ng/ml in Blocker™ Casein in PBS to generate the standard curve for the assay. Following addition of experimental samples or control samples, plates were incubated for 2 hours at room temperature on an orbital shaker. Plates were then washed 3× with 1× washing solution and 50 μL of Sulfo-Tag anti-human/NHP IgG antibody (cat no #D20JL-6, lot no #W0019029S, MSD), at 1/1,000 dilution in Blocker™ Casein in PBS was added to each well and plates were then incubated for 1-1.5 hours at room temperature on an orbital shaker. Plates were washed 3× with 1× washing solution and 150 μL of 2× read Buffer (cat #R92TC-3, MSD) was added to each well. Plates were read immediately on an MSD instrument and the STI-2020 standard curve was used to calculate the concentration of antibody present in serum, lung lavage, and organ lysate materials. Statistical significance was determined using the Welch's t-test.

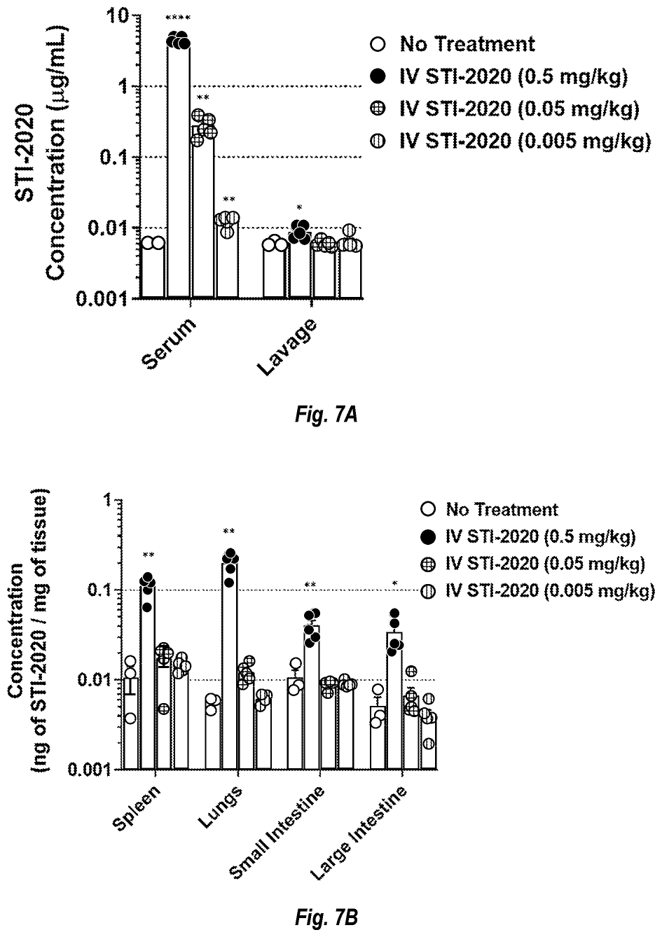

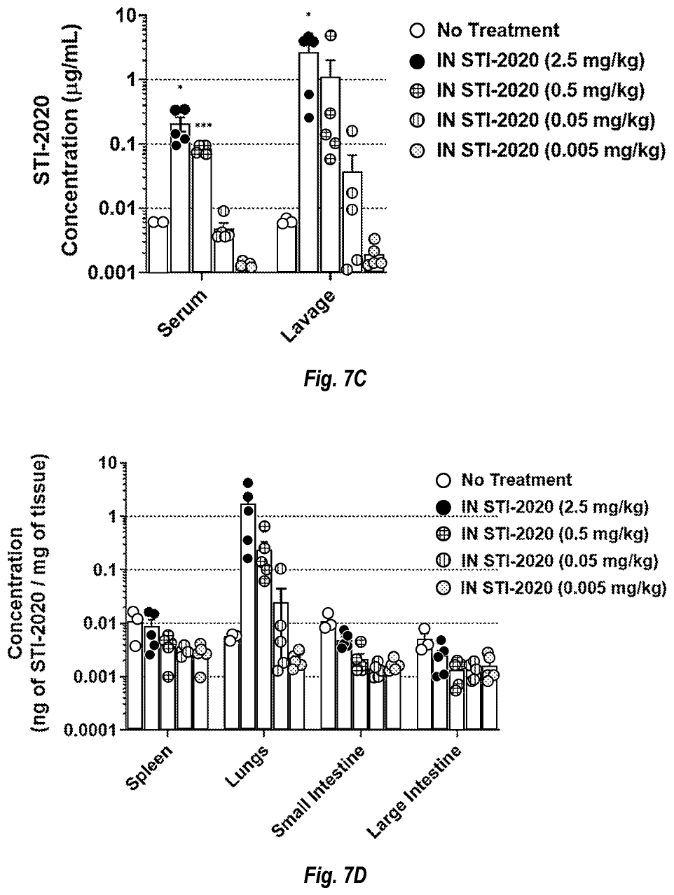

The results of the ELISA antibody quantitation are provided in FIG. 7. Following intravenous (IV) treatment, STI-2020 was detected in the serum, spleen, lungs, small intestine, and large intestine at a dose level of 0.5 mg/kg (FIGS. 7A and 7B). Detected levels in the serum at 0.5 mg/kg dose averaged 4.5 mg/ml, while STI-2020 was present at average concentrations less than 0.01 ug/ml in lung lavage material at each of the IV doses tested (FIG. 7A).

With respect to antibody detected in the organs of animals treated intravenously (FIG. 7B), antibody was detected at a mean concentration of 0.2 ng/mg of processed lung tissue in the 0.5 mg/kg IV dose group. Antibody biodistribution in lung tissue at the 0.05 mg/kg dose level was correspondingly lower, consistent with the 10-fold decrease in the administered dose, and STI-2020 was undetectable in the lungs at the lowest dose, 0.005 mg/kg. Antibody levels in the spleen reached a similar average concentration at 24 hours to that seen in lung tissue (0.1 vs 0.2 ng/mg of tissue, respectively). Antibody was detectable in both the small and large intestines at the highest dose level, with similar average concentrations at 24 hours of 0.04 and 0.03 ng/mg of tissue, respectively.

Following administration of STI-2020 by the intranasal (IN) route (FIGS. 7C and 7D), the concentration of antibody in the serum at 24 h reached an average value of 0.21 mg/ml at the 2.5 mg dose level and was measured at an average value of 0.08 mg/ml in the 0.5 mg/kg dose group (FIG. 7C). As compared to IV treated animals at the 0.5 mg/kg dose, STI-2020 administered intranasally resulted in a 30-fold lower concentration of antibody in serum. In contrast, STI-2020 concentrations in lung lavage samples following IN dosing reached average concentrations of 2.7 mg/ml in the 2.5 mg/kg dose group, and 1.1 mg/ml in animals dosed at 0.5 mg/kg (FIG. 7C). In 4 of 5 treated mice, lung lavage STI-2020 levels following IN administration of antibody at the 0.5 mg/kg dose level were elevated between 6 to 37-fold over those observed in lung lavage following administration of an equivalent IV dose. A single mouse in the IN 0.5 mg/kg group displayed much higher lung lavage distribution of antibody, a likely indication of the variability in delivery efficiency in this experiment (see for example Southam et al. (2002) Am J Physiol Lung Cell Mol Physiol. 282: L833-839, regarding intranasal delivery in the mouse).

STI-2020 was detected in lung tissue samples following an IN dose of 0.5 mg/kg at average concentrations similar to those recorded in IV-treated animals at the same dose level (FIG. 7D). At a dose of 2.5 mg/kg IN, the average concentration in the lung was measured at 0.91 mg/mg of tissue. Besides the antibody detected in lung tissue, STI-2020 levels in spleen, small, intestine, and large intestine at all of the IN dose levels tested did not rise to levels above background in the antibody detection ELISA (FIG. 7D).

### Example 8. Pharmacokinetic Studies of STI-2020 Administered Intranasally to CD-1 Mice

To characterize STI-2020 pharmacokinetics following intranasal dosing at 5 mg/kg, antibody levels in CD-1 mouse lung lysates and serum were quantified at designated timepoints spanning a total of 336 hours (14 days) using a human antibody detection ELISA as described in Example 7.

Female CD-1-IGS (strain code #022) were obtained from Charles River at 6-8 weeks of age. STI-2020 dissolved in intranasal formulation buffer was administered as described for the intranasal biodistribution study in Example 7. Lungs and blood were collected from 3 mice at each of the following timepoints: 10 min, 1.5 h, 6 h, 24 h, 72 h, 96 h, 168 h, 240 h, and 336 h. Serum and lung tissue samples were collected STI-2020 antibody levels in each sample were quantified using the antibody detection ELISA method as described in Example 7. Pharmacokinetic analysis of the collected ELISA data was performed with the Phoenix WiNnonlin suite of software (version 6.4, Certara) using a non-compartmental approach consistent with an intranasal (IN) bolus route of administration and statistical significance was determined using the Welch's t-test.

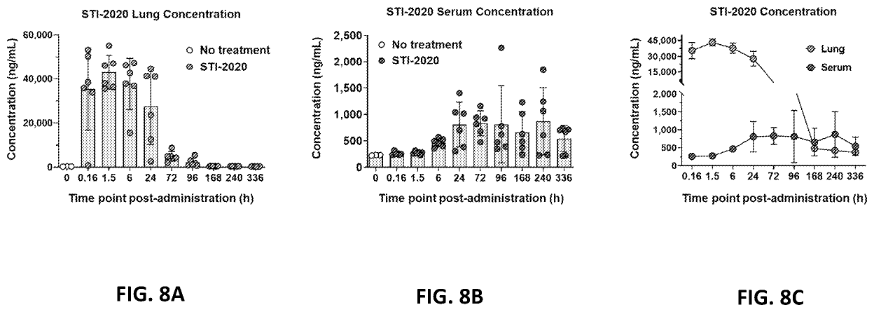

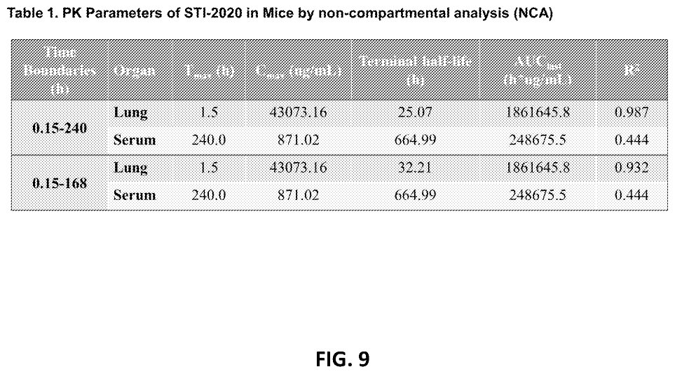

The concentration of STI-2020 in lung lysates and serum for each individual mouse are shown in FIGS. 8A and 8B, respectively. As expected, there were no quantifiable concentrations of STI-2020 antibody in the pre-dose samples. Following IN administration of STI-2020, the antibody concentration was quantifiable up to 240 in serum and up to 336 hours in the lungs. Mouse-to-mouse variability was observed at each time point which could be inherent to the delivery method in the mouse, as during intranasal instillation, the relative distribution between the upper and lower respiratory tract and the gastrointestinal tract is influenced by delivery volume and level of anesthesia (Southam et al. (2002) Am J Physiol Lung Cell Mol Physiol. 282, L833-839). Average antibody concentration in the lung measured 10 minutes after dosing was nearly 70 percent of the maximum antibody concentration (Cmax) measured during the experiment. The Cmax value of STI-2020 in the lungs was measured at 1.5 hours post-administration at a value of 4.3×10⁴ ng/ml. In the lungs, an apparent terminal half-life (TI/2) of 32.21 hours was measured when analyzed between 0.15 and 240 hours (FIG. 9). Under these conditions the R² value equaled 0.932, however when the data were analyzed between 0.15 and 168 hours the R² value increased to 0.987 but the TI/2 dropped to 25.07 hours for the lung samples. In the serum, a TI/2 of 664.99 hours was calculated and coincided with a slow elimination phase (FIGS. 8B and 8C). Kinetics of STI-2020 exposure in the lungs following intranasal administration was accompanied by a slower kinetic of detectable antibody in the serum of treated mice (FIG. 8C). Antibody was first detected in the serum at 6 hours post-administration and the Cmax of 871 ng/ml was detected at the 240-hour timepoint (Tmax). Serum antibody concentrations were within 90% of the recorded Cmax by the 24-hour timepoint. Antibody levels remained relatively constant in serum over the period spanning 24-240 hours, which is in keeping with the calculated STI-2020 serum half-life observed following IV administration of 240 hours in mice. The total systemic exposure (AUCₗₐₛₜ) was significantly higher in the lungs than in the serum of treated mice (AUCₗₐₛₜ were 1,861,645.8 in lungs and 248,675.5 h*ng/mL in serum, FIG. 9). Although some mice may have generated anti-drug antibody (ADA) during the course of the study the data (measured antibody concentrations and PK profiles) do not suggest that PK parameters were significantly influenced by immunogenicity.

### Example 9. Intravenous and Intranasal Delivery of STI-2020 to Treat SARS-CoV-2 Infection in Hamsters

A further experiment was conducted to determine the effects of treating animals with an intranasal formulation of neutralizing antibody STI-2020 (intranasal formulation designated STI-2099) and to compare the effectiveness of intranasal antibody delivery with intravenous antibody delivery in producing a therapeutic effect in treated animals. Based on the observed kinetics of STI-2020 exposure in the lungs following intranasal (IN) dosing at 5 mg/kg (Example 8) and considering the protective efficacy of the 5 mg/kg intravenous (IV) dose in the Syrian golden hamster model of COVID-19 (Example 6), 5 mg/kg was chosen as the IN and IV dose to be administered 12 h post-infection in the hamster SARS-CoV-2 disease model. In this manner, we were able to directly compare the degree of disease severity and duration of disease in animals receiving a therapeutic 5 mg/kg dose of STI-2020 or a control IgG1 antibody (IsoCtl) by either the IV or the IN route. Animals were infected with 5×10⁴ TCID₅₀ of SARS-CoV-2 intranasally and subsequently treated with STI-2020 administered either intravenously or intranasally at 12 hours post-infection. Weight change as a percentage of starting weight was recorded and graphed for each animal.

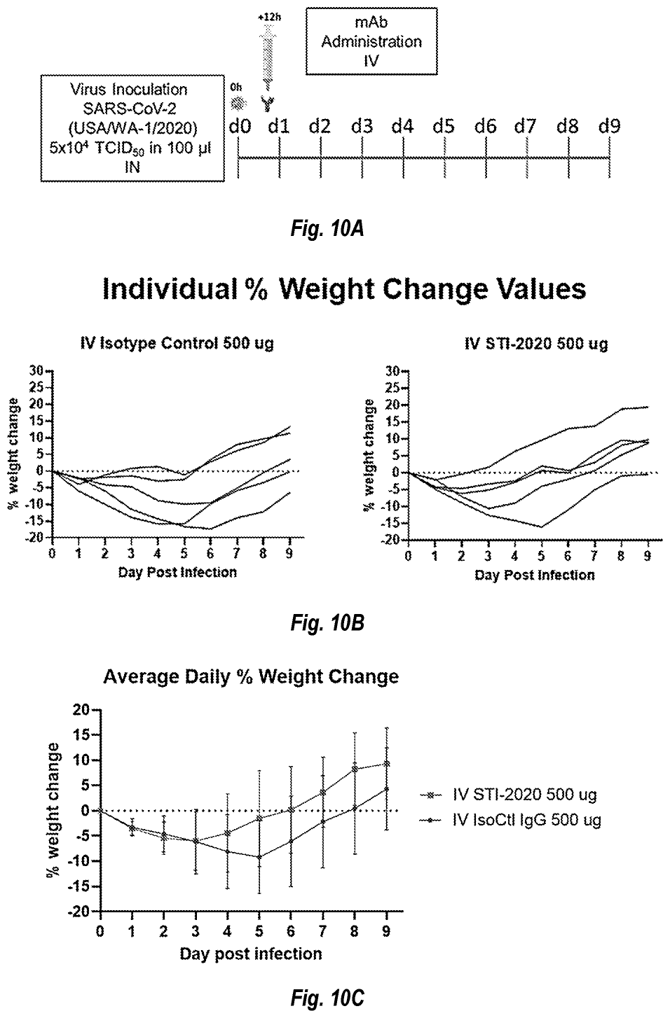

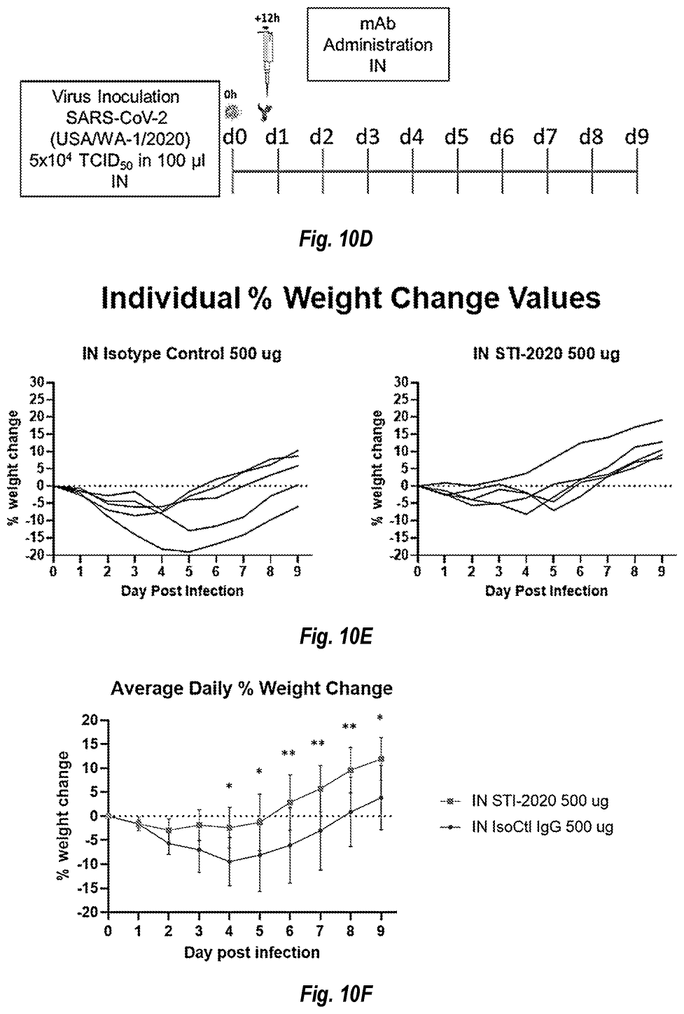

In this experiment six week-old female Syrian golden hamsters (Charles River Laboratories) were infected intranasally with 5×10⁴ median tissue culture does (TCID50) of SARS-CoV-2 in sterile PBS on day 0. Twelve hours post-infection, the animals were treated with a 500 μg dose of neutralizing antibody that was delivered either intranasally (IN), as a liquid composition deposited within the nasal passage, or intravenously (IV). A control isotype antibody was delivered intranasally and intravenously to control animals. Antibody treatments were administered intravenously (IV) with monoclonal antibodies (mAbs) against SARS-CoV-2 S protein, or with an isotype control mAb in up to 350 μl of formulation buffer to anesthetized animals at 12 hours-post inoculation (FIG. 10A). For intranasal delivery of these antibodies, 100 μl of formulated material was introduced directly into the nares and inhaled by anesthetized animals at 12 hours-post viral inoculation (FIG. 10D). The intranasal formulation included antibody in 20 mM Histidine-HCl, 240 mM Sucrose, 0.05% Polysorbate 80, 0.3% Hydroxypropyl Methyl Cellulose (HPMC), pH 5.8. Animals were monitored for illness and mortality for 9 days post-inoculation and clinical observations were recorded daily. Body weights and temperatures were recorded at least once daily throughout the experiment. Average % weight change on each experimental day was compared with the isotype control mAb-treated group using 2-way ANOVA followed by Fisher's LSD test. All animals were housed in animal biosafety level-2 (ABSL-2) and ABSL-3 facilities and studies were conducted according to the National Institutes of Health guidelines.

The data presented in FIG. 10B and FIG. 10C shows that animals administered a 5 mg/kg dose of STI-2020 intravenously experienced a progression of disease similar to that of Isotype Control-treated animals for the first three days of infection. Based on the daily average rate of weight change between the two treatment groups, the STI-2020 treated animals showed a slight decrease in the rate of weight loss between day 2 and day 3. By day 4 of infection, the weight loss rates had further separated along this same trend, and animals in the STI-2020 treatment group had, on average, begun to gain weight. On day 5 of infection, the day on which the maximum average percentage weight loss in the Isotype Control group was observed (9.2%), the STI-2020 animals had already experienced two consecutive days of weight gain (average 2.2 grams/day). Average weight gain between day 3 and day 8 of the experiment between the STI-2020 and the Isotype Control IV-treated groups was 2.8 grams/day and 1.3 grams/day, respectively. IV treatment with STI-2020 12 h post-infection decreased duration clinical signs of disease by at least 24 hours and led to an overall average reduction of disease severity, as manifested by an average rate of weight gain double that of Isotype Control-treated animals between day 3 and day 8 of infection among STI-2020 treated animals, noting that a single animal that experienced a more severe course of the disease than the other four animals in the group (FIG. 10B, right panel).

Animals treated intranasally with STI-2020 experienced a maximum average weight loss of 2.9% of starting weight as compared to a maximum average weight loss level of 9.5% recorded in IN Isotype Control-treated animals. STI-2020-treated animals maintained their average weight over the first four days of infection, while Isotype Control animals steadily lost weight over this timespan (FIG. 10E and FIG. 10F). Beginning on day 4 of infection and extending to day 8 of infection, the weight of STI-2020 treated animals was significantly different than that of animals in the Isotype Control-treated group. By day 8, animals treated IN with STI-2020 had reached an average weight 9.6% above that of their average starting weight, while the average day 8 weight of animals in the Isotype Control group was 0.9% of the average day 0 weight. In general, the difference in average group weights at day 8 reflects both a difference in disease severity in the STI-2020-treated animals between day 0 and day 5 of infection, as well as 20% higher average rate of weight gain among STI-2020-treated animals between days 5 and 8 of infection (3.6 grams/day) vs. that in the Isotype Control-treated group (3 grams/day) (FIGS. 10E and 10F).

Taken together, the animal studies demonstrate the inhibition of disease progression by both intravenous and intranasal delivery of STI-2020, where a noticeable difference in weight loss among intravenously STI-2020-treated animals with respect to isotype antibody-treated controls reached a maximum five days post-infection. For intranasally STI-2020-treated animals, a reduction in weight loss as compared to control animals was evident at 2 days post-infection, and the reduction in this symptom of disease progression with respect to control animals extended through the duration of the study.

Weight loss was the major clinical sign of disease recorded in the experiments with this model, so that reduction in weight loss observed in STI-2020-treated animals with respect to controls represented a mitigating effect of the administered antibody on the severity of disease.

### Example 10. Binding of SID7270 to Cells Expressing SARS-CoV-2 Spike Protein and Spike Protein Variants

To assess ability of the S1D7270 antibodies (which included the LALA mutation in the Fc region) to bind variants of the spike protein, HEK293 cells were transfected using the Fugene reagent with mammalian expression vectors that included either a gene encoding the WA-1 isolate SARS-CoV-2 spike protein or a gene encoding a SARS-CoV-2 variant spike protein having one or more mutations present in SARS-CoV-2 pandemic isolates.

Transformation of HEK293 cells, binding of S1D7270 antibodies to the cells, labeling with fluorescent secondary antibody, and FACS were performed essentially as described in Example 4. Variant spike proteins expressed on the cells included single site mutants A222V, N439K, E484K, F490S, Q493R, S494P, and D614G, a double mutant H49Y/D614G, and a triple mutant K417N, E484K, and N501Y. Further included were the deletion mutants del 69-79, del 141-144, del 210, and del 243-244. The K417N, E484K, and N501Y mutations occur in the B.1.351 “South Africa” SARS-CoV-2 variant. In addition, combination deletion/point mutation mutants that were tested included del 69-79 plus N439K, del 69-79 plus N501Y, del 69-79 plus D796H, and del 69-79 plus N501Y and D614G. The del 69-79 plus N501Y and D614G mutations occur in the B.1.1.7 “UK” SARS-CoV-2 variant. Sequences of variant S proteins are provided herein as SEQ ID Nos. 37-42.

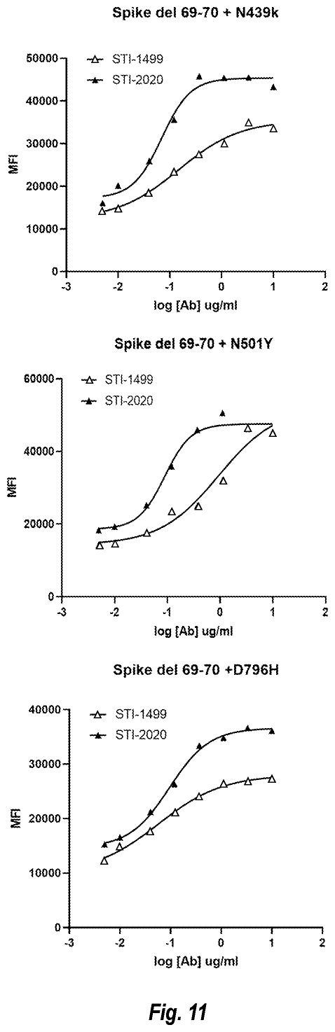

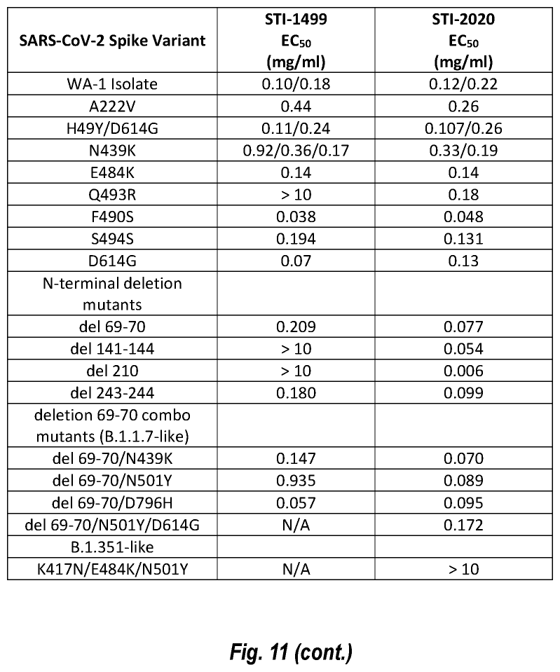

The results in FIG. 11 providing the EC50s for the binding show that the S1D7270 antibody STI-2020 that includes the Fc LALA mutation binds all of the variant SARS-CoV-2 S proteins expressed on the surface of cells. Values separated by a forward slash (/) are from independent experiments. N/A indicates the value was greater than the highest concentration tested and therefore could not be calculated from the values observed within the tested range of concentrations. Also shown are IC50 values for the parental S1D2 antibody having the Fc LALA mutation (STI-1499) binding to the same set of variants. Notably, the STI-1499 and STI-2020 antibodies were found to bind cells expressing the S protein variant for nearly all variants tested.

### Example 11. In Vivo Delivery of DNA and Expression in Muscle

As a test of transformation efficiency by in vivo injection into muscle cells, a luciferase gene was cloned into a Nanoplasmid™ vector (Nature Technology Corp., Lincoln, NE), where the luciferase gene was operably linked to a CMV promoter. The vector was a Nanoplasmid™ (Nature Technology Corp., Lincoln, NE) based on NTC9385R (see, for example, U.S. Pat. No. 9,550,998, herein incorporated by reference) having less than 500 base pairs of bacterial sequences, only three CpG sequences, and lacking antibiotic resistance genes, but instead including an RNAout sequence allowing selection in medium containing sucrose. The heavy and light chain genes each included a 5′ UTR and intron upstream of the coding sequence.

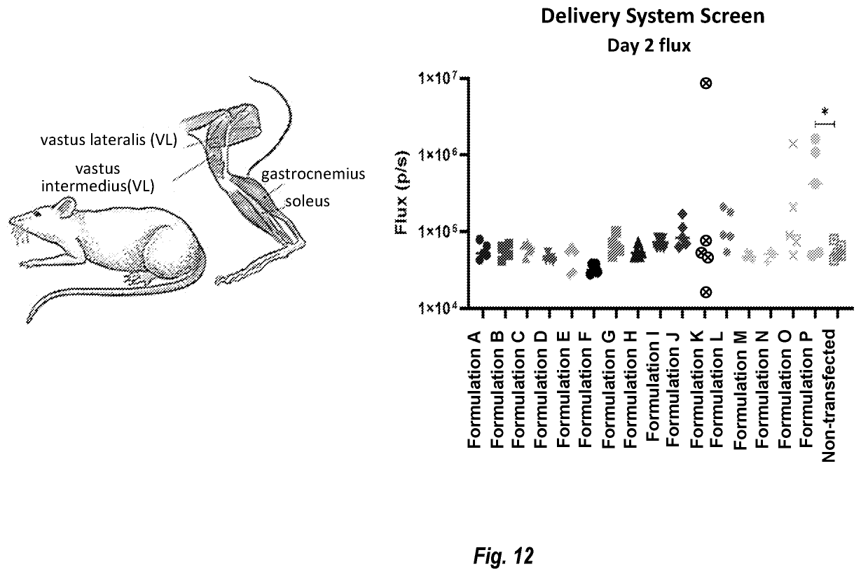

The plasmid was formulated with a variety of transformation reagents, including: polyethylenimine (PEI), complexed at N:P ratios of 2, 4, and 6 (N of PEI versus P of DNA backbone) with the DNA plasmid (formulations A, B, and C, respectively); PEI-mannose, complexed at N:P ratios of 2, 4, and 6 with the DNA plasmid (formulations D, E, and F, respectively); PEI-transferrin, complexed at N:P ratios of 2, 4, and 6 with the DNA plasmid (formulations G, H, and I, respectively); a Fusogenix formulation (Entos Pharmaceuticals, San Diego, CA) (formulation J); a Block Copolymer (Targeted Systems) (formulation K); Block Copolymer (Targeted Systems) plus enhancer agent (formulation L); Block Copolymer (Targeted Systems) plus enhancer agent and condensing agent (formulation M); Sorrento Therapeutics (San Diego, CA) Formulation 4 (formulation N); Sorrento Therapeutics Formulation 25 (formulation O); and Nanotaxi (In Cell Art, Nantes, FR) (formulation P). The formulations were injected into the gastrocnemius muscle of CD-1 mice. The injection dose was 10 μg nanoplasmid. FIG. 12 shows the luciferase readout using an In Vivo Imaging System (IVIS) two days after mice were injected with the nanoplasmid formulations. The graph shows that several of the formulations resulted in luciferase expression in the mice, with particularly strong expression from mice injected with formulations O and P.

### Example 12. In Vivo Delivery of DNA Encoding the SID7270 Antibody and Expression in Muscle

A plasmid was constructed to encode the complete light chain and complete heavy chain of the S1D7270 antibody as separate genes each under the control of a CMV enhancer and an EF1α promoter. The heavy chain gene included an Fc region having the LALA mutation to reduce ADE, and both heavy and light chain antibody genes included sequences encoding N-terminal secretion-enhancement signals. The vector was a Nanoplasmid™ (Nature Technology Corp., Lincoln, NE) based on NTC9385R (see U.S. Pat. No. 9,550,998, herein incorporated by reference) having less than 500 base pairs of bacterial sequences, only three CpG sequences, and lacking antibiotic resistance genes, but instead including an RNAₒᵤₜ sequence allowing selection in culture medium containing sucrose. The heavy and light chain genes each included a 5′ UTR and intron upstream of the coding sequence. The double gene plasmid was purified from E. coli cultures for use in the experiments (see, for example, U.S. Pat. Nos. 9,018,012; 7,943,377; 8,999,672; and 8,748,168 each of which is incorporated by reference in its entirety).

For in vivo transformation of muscle cells, various formulations that included amphiphilic block copolymers and excipients for introducing the plasmid including the genes encoding the S1D7270 antibody were tested.

Formulation 1 included 0.5% poloxamer 181, 4.5% poloxamer 188, and 0.1% DODMA (1,2-dioleyloxy-3-dimethylaminopropane) in Tyrode's solution (137 mM NaCl, 2.7 nM KCl, 1.0 mM MgC₂, 1.8 mM CaCl₂, 20 mM Hepes, and 5.6 nM glucose, pH 7.4). Formula 2 included 0.5% poloxamer 181 and 4.5% poloxamer 188, in Tyrode's solution. Formula 3 included 5% poloxamer 1017 and 0.1% DSPE-PEG2000 (1,2-distearoyl-sn-glycero-3-phosphoethanolamine-N-[amino(polyethylene glycol)-2000]) in Tyrode's solution. Formula 4 included 5% poloxamer 1017 and 0.1% DODMA in Tyrode's solution. Formula 5 included 0.3% poloxamine T/908, 5% poloxamer 188, 0.1% alginate in Tyrode's solution.

Formula 6 included 5% poloxamer 188, 0.2% poly-beta-amino-esters (PBAE) in Tris-buffered saline (TBS). Formula 7 included 3% poloxamine T/1301, 0.3% poloxamer 188, and 0.4% DODMA in TBS. Formula 8 included 3% poloxamine T/1301, 5% poloxamer 407 in TBS. Formula 9 included 3% poloxamine T/1301, 5% poloxamer 188 in TBS. Formula 10 included 3% poloxamine T/1301 and 0.3% poloxamer 407 in TBS. Formula 11 included 3% poloxamine T/1301 and 0.3% poloxamer 188 in TBS.

Each of the tested compositions for plasmid DNA delivery included plasmid DNA at approximately 0.1% of the overall composition.

To test antibody expression in vivo after injection with a nanoplasmid encoding the S1D7270_{LALA} antibody, the nanoplasmid construct that included genes for the S1D7270 light chain and heavy chain were mixed with different compositions as detailed above and injected into the gastrocnemius muscle of mice on day 0. The injection dose was 50 μg nanoplasmid in a volume of 10 to 20 μl. On day 7 and Day 14, blood samples were taken from the injected mice to determine the levels of circulating neutralizing antibody after nanoplasmid delivery. S1D7270_{LALA} antibody was detected by ELISA by detecting anti-human IgG antibody.

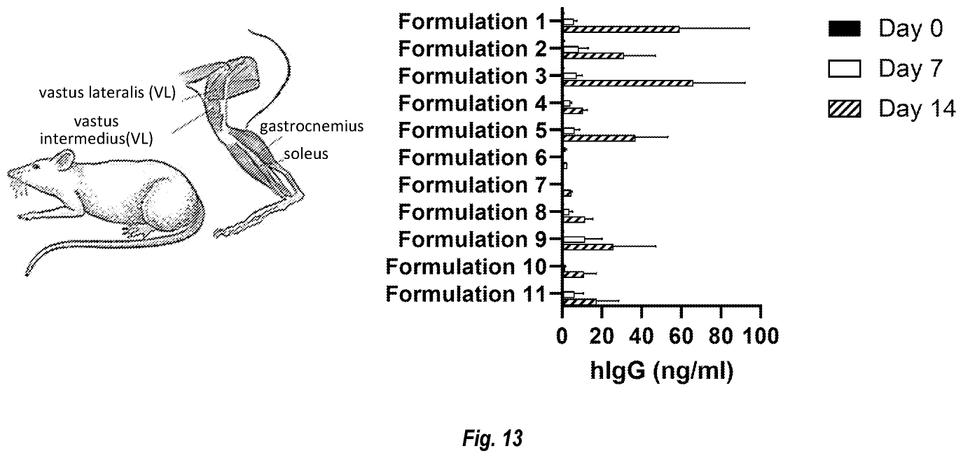

FIG. 13 shows the serum level of S1D7270 antibody at Days 0, 7, and 14. Antibody levels above background (Day 0) were evident at Day 7 for mice injected with nearly all of the formulations, with circulating antibody at Day 14 reaching much higher levels, particularly in mice receiving nanoplasmid in formulations 1, 2, 3, and 5.

### Example 13. Delivery of DNA Encoding STI-2020 Monoclonal Antibody to Mice

Further experiments were performed to demonstrate delivery of DNA encoding the STI-2020 antibody and expression of the antibody. A plasmid was constructed (“pNP-Muscle-S1D7270-HCLC”, SEQ ID NO:36) that encoded the complete light chain (including the light chain variable region of SEQ ID NO:29 and the kappa light chain constant domain) and complete heavy chain of the S1D7270 antibody (including the heavy chain variable region of SEQ ID NO:28 and the heavy chain constant domains) as separate genes under the control of a long desmin promoter (SEQ ID NO:43) and a short desmin promoter (SEQ ID NO:42), respectively. These promoters each included an MCK enhancer sequence followed by the desmin enhancer and included either a long or short desmin promoter sequence which was followed by a pCI intron that was then followed by a Kozak sequence and translational start site. The S1D7270 heavy chain gene included an Fc region having the LALA mutation to reduce ADE, and both heavy and light chain antibody genes included sequences encoding N-terminal secretion-enhancement signals. The vector was a Nanoplasmid™ (Nature Technology Corp., Lincoln, NE) based on NTC9385R (see U.S. Pat. No. 9,550,998, +herein incorporated by reference) which lacked an antibiotic resistance gene but instead including an RNAₒᵤₜ sequence allowing selection in culture medium containing sucrose. The double gene plasmid (pNP-Muscle-S1D7270/HCLC) was purified from E. coli cultures for use in the experiments (see, for example, U.S. Pat. Nos. 9,018,012; 7,943,377; 8,999,672; and 8,748,168 each of which is incorporated by reference in its entirety).

For in vivo transformation of muscle cells, the ICA614 “Nanotaxi” polymer from In-Cell-Art (Nantes, France; see, for example, US Patent Application Publication Nos. 20130177591 and 2010/0179212, U.S. Pat. No. 7,709,452, each of which is incorporated herein by reference in its entirety) was mixed 1:1 with the Nanoplasmid at a concentration to arrive at the correct dosage in the final formulation having a volume of 50 μl.

The study used Tg32 FcRn mice that do not express the mouse Fc receptor but instead are genetically engineered to express the human neonatal Fc receptor to better simulate processing and turnover of the antibody in humans. The nanoplasmid construct that included genes for the S1D7270 mAb light chain and heavy chain (SEQ ID NO:36) was mixed with the ICA614 polymer as detailed above and injected into the gastrocnemius muscle of ten mice per dosage group mice on day 0. The injection dose was 100 μg or 250 μg nanoplasmid in a volume of 50 μl. Following treatment and on days 7, 14, 21, and 28, blood samples were taken from the injected mice to determine the levels of circulating neutralizing antibody after nanoplasmid delivery. S1D7270_{LALA} antibody was detected by ELISA by detecting anti-human IgG antibody. Animal weights were recorded on days 14, 21, and 28, and revealed no significant difference in weight loss between the two dosage groups, and no weight loss in the animals during the course of the study. Mice were sacrificed on day 28 and quadriceps muscle tissue was collected and used for analysis of both antibody and plasmid DNA content. Throughout the study, mice were monitored for morbidity and mortality. No signs of toxicity of the treatment were observed. Two animals of the 250 μg dose group and one animal of the 100 μg dose group died during the course of the study; however the deaths bore no relation to antibody serum level of the animals and there was no indication the deaths related to the antibody treatment.

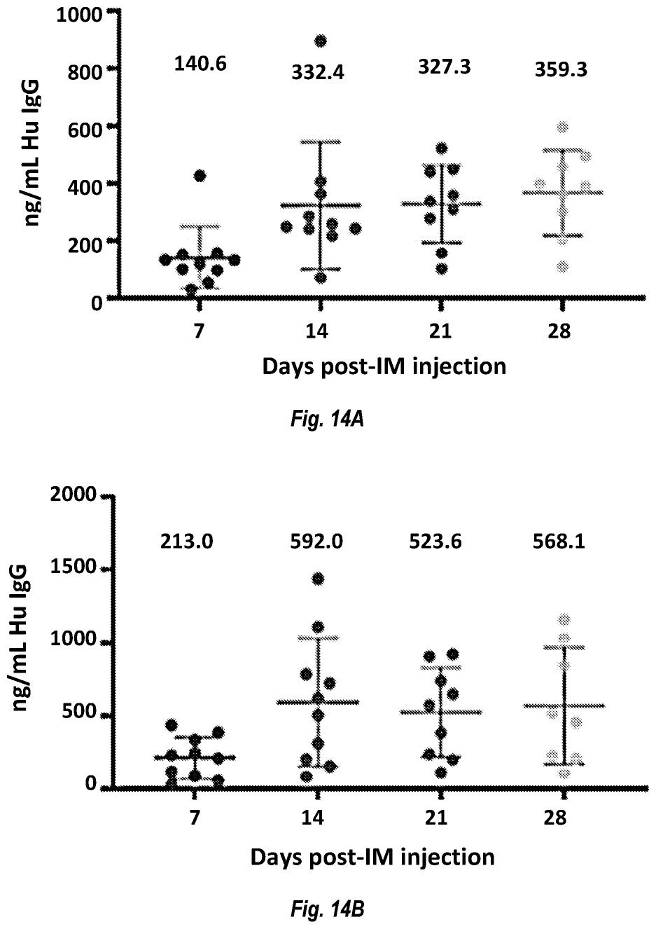

FIG. 14A and FIG. 14B show the serum level of S1D7270 antibody at Days 7, 14, 21, and 28. Antibody levels averaged over 300 ng/ml in the serum of mice receiving 100 μg of the antibody expression plasmid from at least day 14 through day 28 post-treatment and averaged over 500 ng/ml in the serum of mice receiving 250 μg of the antibody expression plasmid over the same time period.

### Example 14. Plaque Reduction Neutralization Test (PRNT) Using Sera from Plasmid-Treated Mice

To assess the neutralization potency of polymer-formulated STI-2020 in the PRNT assay, sera taken from treated animal were tested for their ability to reduce virus plaque formation.

The day before infection, VeroE6 cells were plated in the wells of 24 well tissue culture plates (8×10⁴ cells per well) and incubated at 37° C., 5% CO₂.

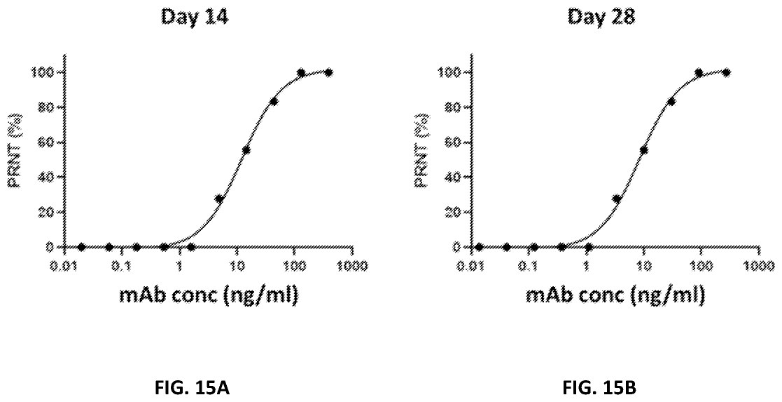

The sera harvested from mice treated as described in Example 13 at Day 14 post-treatment was pooled and then and mixed with the WA1/2020 isolate of SARS-CoV-2 and the pooled sera and virus were incubated for 1 hour at 37° C. The antibody/virus mixtures were then used to infect monolayers of Vero E6 cells grown in the wells the tissue culture plates. Virus supernatant was removed and replaced with fresh medium after 1 hour at 37° C. Cytopathic effect (CPE), i.e., the appearance of plaques or discontinuity in the cell monolayer due to cell lysis, was recorded on day 3 post-infection. At the end of the study, the media was aspirated, and the cells were then fixed with formalin and stained with 0.25% crystal violet. FIG. 15A shows the percent reduction in plaques made by SARS-CoV-2 isolate WA1/2020 with increasing concentration of mouse serum harvested from mice on day 14. The IC₅₀ of the S7E5041_{LALA} antibody in the neutralization assay against the WA1/2020 SARS-CoV-2 WA1/2020 virus was found to be 12.15 ng/mL. FIG. 15B shows the percent reduction in plaques made by SARS-CoV-2 isolate WA1/2020 with increasing concentration of mouse serum harvested from mice on day 28. The IC₅₀ of the S7E5041_{LALA} antibody in the neutralization assay against the WA1/2020 SARS-CoV-2 WA1/2020 virus was found to be 8.3 ng/mL.

### Example 15. Analysis of Plasmid DNA Levels and STI-2020 Antibody Levels in Injected Quadriceps Tissue

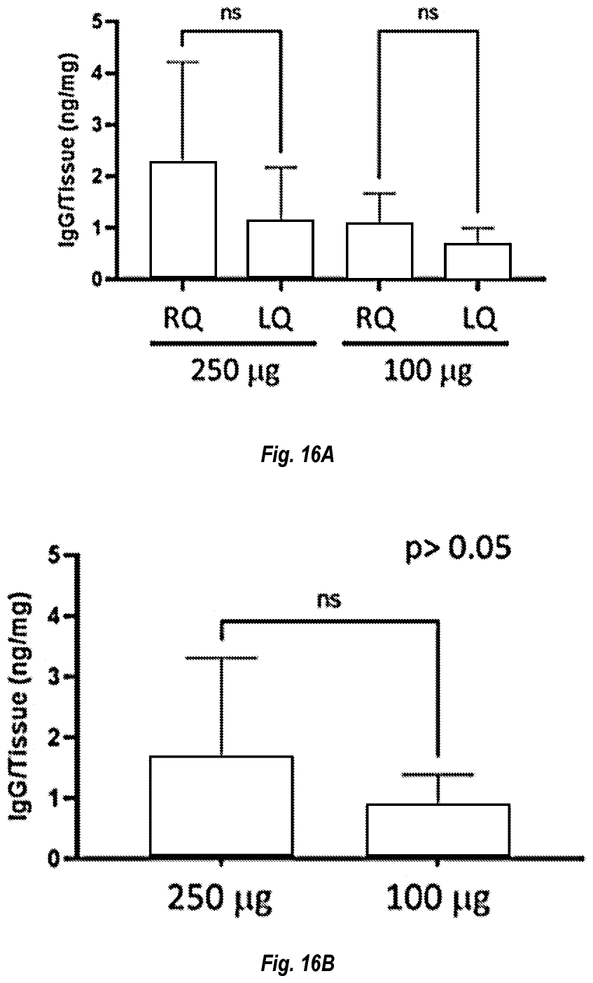

Quadriceps tissue harvested on Day 28 post-treatment (Example 13) were homogenized using a mechanical homogenizer and lysed using a RIPA buffer supplemented with protease inhibitors. The resulting lysates were analyzed using a commercial human IgG ELISA kit (Bethyl Laboratories, Montgomery, TX) to quantify the amount of STI-2020 mAb present in the tissue four weeks after treatment. Human IgG was detected in muscle tissue 28 days after injection with either 100 μg or 250 μg the plasmid DNA (FIGS. 16A and 16B). FIG. 16A illustrates that there was no significant difference in the amount of antibody detected in the left and right quadriceps muscles and FIG. 16B demonstrates the difference in the amount of antibody in the 100 μg or 250 μg plasmid treatments (pooled right and left quadriceps tissue) was not significant.

Homogenized day 28 tissue from each treated animal was also used in PCR designed to detect plasmid DNA in the tissue, however, using a housekeeping gene served as a positive control, no plasmid DNA was detected above the detection limit of 300 nanoplasmid copies.

### Example 16. Binding of SID7270 to Cells Expressing Additional SARS-CoV-2 Spike Protein Variants

To assess ability of the S1D7270 antibodies (which included the LALA mutation in the Fc region) to bind further variants of the spike protein, HEK293 cells were transfected using the Fugene reagent with mammalian expression vectors that included either a gene encoding a SARS-CoV-2 Delta variant S protein and a SARS-CoV-2 Delta variant S protein. The Delta variant S protein had the following mutations with respect to the WA1/2020 SARS-CoV-2 S protein: T19R, T95I, G142D, deletion of 157-158, A222V, L452R, T478K, D614G, P681R, and D950N. The Delta Plus variant S protein had the following mutations with respect to the SARS-CoV-2 S protein: T19R, T95I, G142D, deletion of 157-158, A222V, K417N, L452R, T478K, D614G, P681R, and D950N.

Transformation of HEK293 cells, binding of S1D7270 antibodies to the cells, labeling with fluorescent secondary antibody, and FACS were performed essentially as described in Example 4.

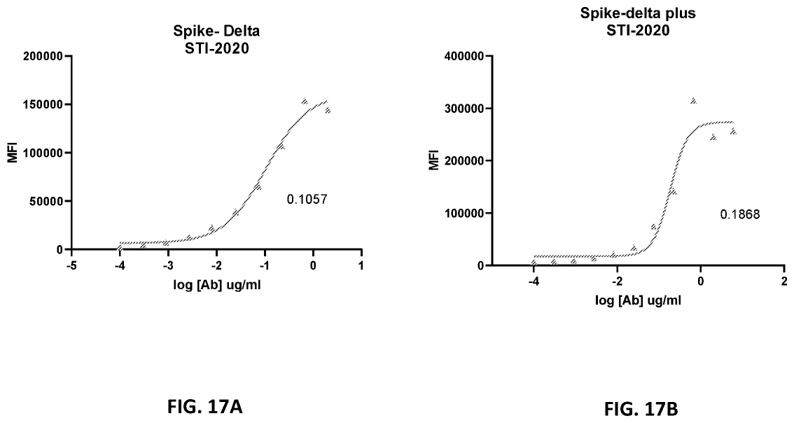

The results in FIG. 17 providing the EC50s for the binding show that the S1D7270 antibody that includes the Fc LALA mutation (STI-2020) binds both of the variant SARS-CoV-2 S proteins expressed on the surface of cells. In this assay, STI-2020 was found to have an EC50 for the Delta variant S protein of 0.105 μg/ml and an EC50 for the Delta Plus variant S protein of 0.1868 μg/ml.

### Example 17. S7E5041_{LALA} Neutralization Assay Using Pseudotyped Viruses Displaying Engineered Mutant S Proteins

S protein genes encoding S protein mutants were constructed and cloned into plasmid pCDNA3.1 (ThermoFisher). Codon optimized SARS-CoV-2 Wuhan Spike carrying the D614G amino acid change (Sino Biological #VG40589-UT(D614G)) was modified to remove the last 21 amino acids at the C-terminus (SpikeA21) and was used as the parental clone into which mutations were introduced. To generate pseudotyped viruses displaying mutant S proteins, 2 μg of Spike plasmid was transfected into BHK21 cells with 2 μg of Spike plasmid using an Amaxa Nucleofector II with cell line kit L (Lonza #VCA-1005) and program A-031. Twenty-four hours later, the cells were transduced with VSV deleted for the G gene and encoding a firefly luciferase gene (VSVΔG(FLuc) at MOI of about 4 for 1 hour at 37° C. after which the medium containing the VSVΔG(FLuc) was removed. The cells were washed 2× with DPBS prior to the addition of fresh culture medium. After 24-44 hrs, culture supernatants containing pseudotyped viruses displaying the mutant S protein expressed by the host cells were collected, centrifuged at 300×g for 5 min at room temperature, aliquoted, and frozen.

For neutralization assays, the pseudotyped viruses were first normalized for fluorescence intensity on 293-ACE2 cells by luciferase assay. The VSVΔG(FLuc)-S pseudotypes were incubated with the indicated concentrations of anti-spike antibody as well as 1 μg/mL of anti-VSV-G antibody which was added to neutralize any remaining parental VSV in the virus preparation for 30 minutes at room temperature. The VSVΔG(FLuc)-S pseudotype plus antibody was then added to 293-ACE2 cells in 96- or 384-well format, and luciferase output was measured after 40-48 hrs. Relative IC50 was determined using nonlinear fit variable slope (4PL), and absolute IC50 was determined using nonlinear fit with a top constraint of 100 (complete inhibition) and a baseline constraint of 0 (no inhibition).

293-ACE2 cells were plated to white-walled 96-well plates at 40K cells/well and incubated at 37° C./5% CO₂. The next day, pseudotyped VSV was incubated with anti-spike (concentration as indicated) and anti-VSV-G (1 μg/mL) antibodies for 30 minutes at room temperature and added to the 293-ACE2 cells in triplicate. Transduced cells were incubated for 24 hours and luminescence measured using a Tecan Spark plate reader. The percent inhibition was calculated using 1-([luminescence of antibody treated sample]/[average luminescence of untreated samples])×100. Absolute IC50 was calculated using non-linear regression with constraints of 100 (top) and 0 (baseline) using GraphPad Prism software. At least 6 replicates over 2 independent experiments were included. Negative value slopes were assigned IC50 of >10 p g/mL.

Table 1 shows the results of these neutralization assays performed with multiple versions of pseudotyped VSVΔG(FLuc)-S having different S protein mutations and the STI-2020 antibody. Although the STI-2020 antibody did not demonstrate specific inhibition of infection by viruses expressing the Beta and Gamma S variants, the STI-2020 antibody did show neutralizing activity against viruses expressing the Delta variant as well as Alpha, Epsilon, Kappa, and Zeta S protein variants, and to a lesser extent, the Delta Plus S protein variant. The sequences of full-length S proteins of the Alpha, Beta, Gamma, Delta, Delta Plus, and Kappa SARS-CoV-2 variants are provided as SEQ ID Nos:37-42.

TABLE 1
IC50 of STI-2020 Inhibition of Infection
of Variant S Protein-pseudotyped Viruses

| | | STI-2020 |
|---|---|---|
| | | Antibody |
| | | IC₅₀ |
| SAR-CoV-2 Spike Variant | Variant lineage | (μg/ml) |
| D614G | | 0.0121 |
| Δ69/70-Δ144-N501Y-A570D- | B.1.1.7 (U.K., Alpha) | 0.1882 |
| D614G-P681H-T716I-S982A- | | |
| D1118H | | |
| D80A-D215G-Δ242/244- | B.1.351 (South Africa, Beta) | |
| K417N-E484K-N501Y- | | |
| D614G-A701V | | |
| D614G, S13I, W152C, L452R | B.1.429 (California, Epsilon) | 0.0069 |
| G142D, E154K, L452R, | B.1.617.1 (India, Kappa) | 0.0387 |
| E484Q, D614G, P681R, | | |
| Q1071H, H1101D | | |
| T19R, (G142D), Δ156-157, | B.1.617.2 (India, Delta) | 0.0394 |
| R158G, L452R, T478K, | | |
| D614G, P681R, D950N | | |
| T19R, (G142D), Δ156-157, | B.1.617.2.1 (AY.1, Delta | 0.5752 |
| R158G, K417N, L452R, | Plus) | |
| T478K, D614G, P681R, | | |
| D950N | | |
| L18F, T20N, P26S, D138Y, | P.1 (Brazil/Japan, Gamma) | |
| R190S, K417T, E484K, | | |
| N501Y, D614G, H655Y, | | |
| T1027I | | |
| E484Q, F565L, D614G, | P.2 (Brazil, Zeta) | 0.0344 |
| V1176F | | |
| Δ69/70, D614G, N501Y | B.1.1.7 (U.K., Alpha) | |
| D614G, K417N, E484K, | B.1.351 (South Africa, Beta) | |
| N501Y | | |
| L452R, E484Q, P681R | B.1.617.1/3 (India, Kappa) | 0.0395 |
| D614G, L452R, E484K | | 0.0517 |

### Example 18. Plaque Reduction Neutralization Test Assays with Live SARS-CoV-2

In vitro assays were performed to assess the ability of the STI-2020 antibody to block infection of susceptible cells by the SARS-CoV-2 WA-1/2020 isolate and the Alpha, Beta, Delta variant and the SARS-CoV-2 Delta Plus variant. The day before infection, VeroE6 cells were plated in the wells of tissue culture plates and incubated at 37° C., 5% CO₂. The STI-2020 antibody was serially diluted in culture media and equal volumes of the serially diluted antibody samples and SARS-CoV-2 virus (either the Delta or Delta Plus variant) were combined and incubated for 1 hour at 37° C. Two different preparation batches of the STI-2020 antibody were tested. The antibody/virus mixtures were then used to infect monolayers of Vero E6 cells grown in the wells the tissue culture plates. Virus supernatant was removed and replaced with fresh medium after 1 hour at 37° C. Cytopathic effect (CPE), i.e., the appearance of plaques or discontinuity in the cell monolayer due to cell lysis, was recorded on day 3 post-infection. At the end of the study, the media was aspirated, and the cells were then fixed with formalin and stained with 0.25% crystal violet.

Based on the percent reduction in plaques made by SARS-CoV-2 variants with increasing concentration of antibody, the IC₅₀ value of the STI-2020 antibody in the neutralization assay against the WA1/2020 virus was found to be 43.33 ng/mL (STI-2020 batch 1) and 64.47 ng/mL (STI-2020 batch 2). Interestingly, in the live SARS-CoV-2 infection assay, the IC50 for neutralizing some of the variant viruses, particularly the Alpha (“UK”) virus and the Delta (“India”) virus, was lower than the IC50 for neutralizing the original WA-1/2020 isolate. The IC50 value of the STI-2020 antibody in the neutralization assay against the Alpha variant virus was found to be 9.076 ng/mL and 16.42 ng/mL. The IC₅₀ value of the STI-2020 antibody in the neutralization assay against the Delta variant virus was found to be 6.06 ng/mL and 33.28 ng/mL. The STI-2020 antibody did not effectively neutralize the Beta variant virus or the Gamma variant virus in the PRNT assay.

1. An isolated antigen-binding protein that specifically binds the spike (S) protein of SARS-CoV-2, wherein the antigen-binding protein comprises a heavy chain variable domain having at least 95% sequence identity to the amino acid sequence of SEQ ID NO:28 and a light chain variable domain having at least 95% sequence identity to the amino acid sequence of SEQ ID NO:29.

2. An isolated antigen-binding protein that specifically binds the S protein of SARS-CoV-2, comprising a heavy chain complementarity determining region (CDR) 1 having the amino acid sequence of SEQ ID NO:30, a heavy chain CDR2 having the amino acid sequence of SEQ ID NO:31, a heavy chain CDR3 having the amino acid sequence of SEQ ID NO:32, a light chain CDR1 having the amino acid sequence of SEQ ID NO:33, a light chain CDR2 having the amino acid sequence of SEQ ID NO:34, and a light chain CDR3 having the amino acid sequence of SEQ ID NO:35.

3. The isolated antigen-binding protein according to claim 2, wherein the heavy chain variable region comprises an amino acid sequence having at least 95% identity to the amino acid sequence of SEQ ID NO:28 and the light chain variable region comprises an amino acid sequence having at least 95% identity to the amino acid sequence of SEQ ID NO:29.

4. The isolated antigen-binding protein according to claim 3, wherein the heavy chain variable region comprises the amino acid of SEQ ID NO:28 and the light chain variable region comprises the amino acid sequence of SEQ ID NO:29.

5. The isolated antigen-binding protein of claim 1, wherein the antigen-binding protein binds the S protein of SARS-CoV-2 with a dissociation constant (K_{d}) of 10⁻⁷ M or less.

6. The isolated antigen-binding protein of claim 4, wherein the antigen-binding protein binds the S protein of SARS-CoV-2 with a K_{d} of 10⁻⁸ M or less.

7. The isolated antigen-binding protein of claim 1, wherein the antigen-binding protein is an antibody or antibody fragment.

8. The isolated antigen-binding protein of any of claim 1, wherein the antigen-binding protein is a fully human antibody, comprises a heavy chain variable region and a light chain variable region of a fully human antibody, or comprises an antibody fragment derived from a fully human antibody.

9. The isolated antigen-binding protein of claim 1, comprising an IgG1, IgG2, IgG3, or IgG4 antibody.

10. The isolated antigen-binding protein of claim 9, comprising an IgG1 or IgG4 antibody.

11. The isolated antigen-binding protein of claim 10, wherein the IgG1 or IgG4 antibody comprises a mutant Fc region.

12. The isolated antigen-binding protein of claim 11, wherein the Fc region comprises at least one mutation that decreases ADE, or wherein Fc region comprises the mutations L234A and L235A (LALA), or wherein the Fc region comprises at least one mutation that increases antibody half-life, or wherein Fc region comprises the mutations M252Y, S254T, and T256E (YTE).

13. (canceled)

14. (canceled)

15. (canceled)

16. The isolated antigen-binding protein of claim 1, wherein the antigen-binding protein is a Fab fragment, a Fab′ fragment, or a F(ab′)2 fragment or wherein antigen-binding protein is a single chain antibody (scFv or scFab).

17. (canceled)

18. The isolated antigen-binding protein of claim 1, wherein the antigen-binding protein is a neutralizing antibody or antigen-binding fragment thereof that blocks binding of the SARS-CoV-2 S protein to the ACE2 protein.

19. (canceled)

20. (canceled)

21. (canceled)

22. (canceled)

23. (canceled)

24. A pharmaceutical composition, comprising the antigen-binding protein of claim 1 and a pharmaceutically-acceptable excipient.

25. The pharmaceutical composition of claim 24, wherein the antigen-binding protein is an IgG antibody, a Fab, Fab′ or F(ab′)2 fragment, or a single chain antibody.

26. (canceled)

27. (canceled)

28. (canceled)

29. (canceled)

30. (canceled)

31. (canceled)

32. A method for treating a subject having or suspected of having a coronavirus infection, the method comprising: administering to the subject an effective amount of the pharmaceutical composition of claim 24 to a subject infected with or suspected of being infected with a coronavirus.

33. (canceled)

34. (canceled)

35. (canceled)

36. (canceled)

37. (canceled)

38. (canceled)

39. (canceled)

40. (canceled)

41. A composition comprising at least one nucleic acid molecule that comprises a nucleic acid sequence encoding a polypeptide comprising a heavy chain variable region having at least 95% identity to SEQ ID NO:28 and a nucleic acid sequence encoding a polypeptide comprising a light chain variable region having at least 95% identity to SEQ ID NO:29.

42. (canceled)

43. A host cell harboring the nucleic acid molecule or vector as defined in claim 41.

44. A method of treating or preventing a coronavirus infection, comprising:
administering to a subject having or suspected of having a coronavirus infection, or at risk of becoming infected with a coronavirus, at least one nucleic acid construct encoding an antibody having a light chain variable sequence having at least 95% identity to SEQ ID NO:28 and a heavy chain variable sequence having at least 95% identity to SEQ ID NO:29.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)
